(19) United States
(12) Patent Application Publication
(10) Pub. No.: US 2023/0255884 A1
(43) Pub. Date: Aug. 17, 2023

(54) FINISHED FIBROUS SRUCTURES AND METHODS OF THEIR USE AND PREPARATION

(71) Applicant: CLEXIO BIOSCIENCES LTD., Jerusalem (IL)

(72) Inventors: Erez KOREN, Givat Shaul (IL); Richard FISHER, Givat Shaul (IL)

(21) Appl. No.: 17/607,688

(22) PCT Filed: May 1, 2020
(86) PCT No.: PCT/IB2020/054138

§ 371 Date: Oct. 29, 2021

Related U.S. Application Data
(60) Provisional application No. 62/841,373, filed on May 1, 2019.

(51) Int. Cl. A61K 9/00; A61K 31/167; A61K 47/26; A61K 31/196; A61K 31/4174; A61K 31/192; A61K 47/10

(52) U.S. Cl. CPC A61K 9/0092; A61K 31/167; A61K 47/26; A61K 31/196; A61K 31/4174; A61K 31/192; A61K 47/10

(57) ABSTRACT

The invention is directed to fibrous structures finished with active pharmaceutical ingredients, as well as methods of their manufacturer and use.

### CROSS REFERENCE TO RELATED APPLICATIONS

This application claims the benefit of U.S. Provisional Application No. 62/841,373, filed May 1, 2019, the entirety of which is incorporated by reference herein.

### TECHNICAL FIELD

The invention is directed to fibrous structures finished with active pharmaceutical ingredients, as well as methods of their manufacturer and use.

### BACKGROUND

The transdermal and dermal administration of active pharmaceutical ingredients is known. Transdermal and dermal formulations include, for example, gels, creams, sprays, and lotions that are applied to the skin. Other formulations include patches that are affixed to the skin using an adhesive. While these formulations are useful, there are occasions where a gel, cream, spray, ointment, foam or lotion might be too messy or greasy, resulting in a significant amount of the formulation being transferred away from the skin. In addition, in order to maintain efficacy, these types of formulations required two, three, or more applications, per day. A disadvantage of patch formulations, which is due to their inflexibility, is that there are areas of the body wherein a patch does not adhere well, for example on joint areas or areas having skin-to skin contact such as between fingers or toes. Patch use can also be limited to those areas with intact skin or non-irritable skin, since the adhesives can make removal damaging and/or painful.

Dyeing is a time honored, mature technology of aqueous application of color to fibrous structures, mainly using synthetic organic dyes and frequently at elevated temperatures and pressures in some of the steps. It is generally accepted that there is no dye which dyes all existing fibers and no fiber which can be dyed by all known dyes. The interaction between the dye and the fiber is related to their different chemical and physical characteristics. The desired interaction is dictated by the two main goals of textile dyeing technology, namely to obtain some affinity of the dyestuff to the fiber to promote transfer from the solvent medium to the fiber and to obtain a fixation of the dye to the fiber in order to achieve extraction resistance, known in the art as fastness. During dyeing, the dyes, possibly along with chemical aids such as surfactants, acids, alkali/bases, electrolytes, carriers, leveling agents, promoting agents, chelating agents, emulsifying oils, softening agents etc. . . . are applied to the textile to obtain a uniform depth of color and the color fastness properties suitable for the end use. Usually, this means satisfactory fastness to laundry, soaking, perspiration and abrasion. The dyeing process includes dispersion of the dye in the aqueous media, diffusion of the dye in the liquid phase followed by adsorption onto the outer surface of the fibers and finally diffusion and adsorption to the inner bulk of the fibers. Fastness properties are always required to render the textile fit for use. Surface coloration can also be accomplished by applying pigments (pigments differ from dyes by not showing chemical or physical affinity for the fibers) together with adhesives (polymers which fix the pigment to the fibers). This process is also common in textile printing.

The long established technology of dyeing of hydrophobic fibers with disperse dyes, is a good example of a commercial application of a non-soluble material to a fibrous substrate, which is well known to those versed in the art. Disperse dyes are stable, chemically inert pigments that can be milled to micron size to enhance their dispensability and dispersion stability, so that phase separation and/or aggregation and/or crystallization is minimal. The maximum amount of dye weight per fiber weight add-on required to obtain deep shades is usually under 10% (w/w). Disperse dyes diffuse into the polymer matrix and are therefore very durable to aqueous extraction. The relatively low add-on and full penetration into the polymer matrix result in minimal impact on the textile's functional and aesthetic properties. Such commercially practiced procedures are inadequate however to produce a useful finishing of a textile substrate with an active pharmaceutical ingredient (API). APIs are chemically active compounds which are typically both pH and heat sensitive and have a strong tendency to form large crystalline structures and phase separate from a dispersion or emulsion. Additionally, add-ons that exceed 10% (w/w) are usually required to obtain the desired therapeutic effects. Furthermore, the durability to extraction, sought in conventional practiced procedures, is actually a negative property for the purpose of transferring the API to the patient.

New methods for transdermally and dermally administering active pharmaceutical ingredients are needed.

### SUMMARY

The invention is directed to dry, non-occlusive, pharmaceutical fibrous structures finished with an active pharmaceutical ingredient, or pharmaceutically acceptable salt thereof. Methods of making and using the finished, dry, non-occlusive, pharmaceutical fibrous structures are also described.

### BRIEF DESCRIPTION OF THE DRAWINGS

FIG. 1A depicts final fibrous structure weight as compared to original fibrous structure weight, prepared using methods of the invention.

FIG. 1B depicts final fibrous structure weight as compared to original fibrous structure weight, prepared using methods of the invention.

FIG. 2 depicts the percentage of API released over time, as a function of pH.

FIG. 3 depicts mean lidocaine finishing of different fibrous structures, using methods of the invention.

FIG. 4A depicts mean lidocaine plasma concentration versus time for embodiments of the invention, as compared to a reference.

FIG. 4B depicts mean lidocaine plasma concentration over time for embodiments of the invention, as compared to a reference.

FIG. 4C depicts mean lidocaine plasma concentration over time for embodiments of the invention, as compared to a reference.

FIG. 5 depicts mean lidocaine skin concentrations after exposure to embodiments of the invention, as compared to a reference.

FIG. 6 depicts mean lidocaine plasma concentration over time for an embodiment of the invention.

FIG. 7 depicts mean lidocaine plasma concentration over time for an embodiment of the invention.

FIG. 8 depicts mean lidocaine plasma concentration over time for an embodiment of the invention.

FIG. 9A depicts mean lidocaine skin concentrations after exposure to embodiments of the invention, as compared to a reference.

FIG. 9B depicts mean lidocaine stratum corneum concentrations after exposure to embodiments of the invention, as compared to a reference.

FIG. 10 depicts mean lidocaine plasma concentration over time for an embodiment of the invention.

FIG. 11 depicts mean lidocaine skin concentrations after exposure to embodiments of the invention for 96 h, as compared to a reference.

FIG. 12A depicts percentage Add-on using increasing concentrations of lidocaine in finishing compositions at 2-8° C., 50° C., 80° C., and 25° C.

FIG. 12B depicts scanning electron microscope pictures of 80° C. finished and unfinished fibrous structures.

FIG. 13 depicts percentage Add-on of lidocaine, without added excipients in the finishing composition.

FIG. 14A depicts loading kinetics sample shape A.

FIG. 14B depicts loading kinetics sample shape B.

FIG. 14C depicts loading kinetics sample shape C.

FIG. 14D depicts loading kinetics sample shape D.

FIG. 15 depicts percentage Add-on with increasing loading time.

FIG. 16 depicts lidocaine base content with increasing concentration of surfactant.

FIG. 17 depicts mean percentage Add-on with increasing pH.

FIG. 18A depicts 3% lidocaine sample dissolution profiles

FIG. 18B depicts 3% lidocaine sample dissolution profiles (initial 10 minutes)

FIG. 18C depicts 10% lidocaine sample dissolution profiles

FIG. 18D depicts 10% lidocaine sample dissolution profiles (initial 10 minutes)

FIG. 18E depicts 25% lidocaine sample dissolution profiles

FIG. 18F depicts 25% lidocaine sample dissolution profiles (initial 10 minutes)

FIG. 19 depicts stiffness with increasing percentage of Add-on.

FIG. 20A depicts force peak with increasing percentage Add-on.

FIG. 20B depicts percentage strain peak with increasing percentage Add-on.

FIG. 21 depicts evaporation % weight loss at 72 hours.

### DETAILED DESCRIPTION OF ILLUSTRATIVE EMBODIMENTS

The present invention may be understood more readily by reference to the following detailed description taken in connection with the accompanying figures and examples, which form a part of this invention. It is to be understood that this invention is not limited to the specific compositions or methods described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. All ranges are inclusive and combinable.

The modifier “about” should be considered as disclosing the range defined by the absolute values of the two endpoints. For example, the expression “from about 2 to about 4” also discloses the range “from 2 to 4.” When used to modify a single number, the term “about” may refer to plus or minus 10% of the indicated number and includes the indicated number. For example, “about 10%” may indicate a range of 9% to 11%, and “about 1” means from 0.9 to 1.1.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any sub-combination. Further, reference to values stated in ranges includes each and every value within that range.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like.

“Subject” includes both humans and other mammals, including large and small animals such as dogs, cats, rabbits, horses, cows, pigs, and the like. The terms “patient” and “subject” are used interchangeably herein. Preferably, the subject is a human.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” of any disease or disorder refers to ameliorating at least one physical parameter, which may not be discernible by the subject. In yet another embodiment, “treating” or “treatment” of any disease or disorder refers to modulating the disease or disorder, either physically, (e.g., stabilization of a discernible symptom), physiologically, (e.g., stabilization of a physical parameter), or both. In yet another embodiment, “treating” or “treatment” of any disease or disorder refers to delaying the onset of the disease or disorder.

“Hypoallergenic,” as used herein, refers to a product of the invention that is unlikely to elicit an allergy response in a subject exposed to the product.

“Non-irritant,” as used herein, refers to a product of the invention that is unlikely to elicit an inflammatory and/or painful response in a subject exposed to the product.

“Therapeutically effective amount” as used herein refers to an amount of active pharmaceutical ingredient effective to exert the intended therapeutic action in a subject.

According to the invention, “finishing” is a process (i.e., a treatment) whereby one or more materials are caused to interact with a fibrous structure both by diffusion into the polymer comprising the fibers and/or by deposition onto the surfaces of the fibers and/or the interstices between the fibers.

According to the invention, “fibrous structures” are structures composed of either loose fibers, yarns, fabric (e.g., woven, knit, braided or non-woven fabrics) and comprised of natural fibers, manmade fibers, synthetic fibers, or mixtures thereof. In some embodiments, the fibrous structure is a woven fibrous structure comprised of natural fibers, manmade fibers, synthetic fibers, or mixtures thereof. In other embodiments, the fibrous structure is knit fibrous structure comprised of natural fibers, manmade fibers, synthetic fibers, or mixtures thereof. In other embodiments, the fibrous structure is non-woven fibrous structure comprised of natural fibers, manmade fibers, synthetic fibers, or mixtures thereof. The term “fibrous structures” is understood to relate to loose fibers, yarns, fabrics, garments and other such complete textiles.

According to the invention, “time period” refers to the duration of time the dry, non-occlusive, fibrous structures finished with a finishing composition of the invention are continuously or intermittently applied to the subject's intact skin.

Applying a substance to a fibrous structure, often referred to as “finishing,” is a complex endeavor. Usually, the process is described in terms of interconnected, yet independently controlled steps: homogeneous dispersion of the substance in a liquid medium which can flow around and contact all the fiber surfaces of the structure, migration of the dispersed substance particles from the dispersion's bulk onto the fiber surfaces, interaction of the particles with the fiber surfaces, and optionally, diffusion of the particles into the polymer matrix of the fiber and, finally, fixation of the substance in the fibrous structure to prevent it from being easily detached, once the process is completed. “Homogeneous dispersions,” within the scope of this disclosure, include, for example, solutions, colloids, stable colloids, suspensions, and emulsions.

Homogeneous dispersion requires a suitable solvent to create a true solution. Alternately, if the substance is insoluble in the chosen solvent, then a stable colloid must be created by reduction of particle size and judicious use of surfactants, wetting agents, and stabilizers. Separation, sedimentation, crystallization, and aggregation should be prevented. Preferential migration of the substance onto the fiber surfaces requires an affinity to exist between the dispersed particles and the fibers. This affinity may be ionic, electrostatic, polar, Van der Waals, or hydrophobic/hydrophobic interaction. Affinity is required for an efficient process, otherwise much of the substance will remain in the bulk of the dispersion. Affinity cannot be too strong or an un-level finish may result. Once in contact with the fiber surface, the substance must preferentially interact with it, otherwise, it will randomly return to the dispersion bulk. Penetration of the substance into the fiber's polymer matrix depends on its ability to diffuse into the specific polymer. Fixation also depends on the chemical and physical characters of the substance and polymer.

There are many variables and multiple combinations of the variables which can affect the successful completion of a finishing process. A specific method must be developed for each unique fiber-substance pair. The chemical and physical characteristics of the substance and fiber involved affect the types of possible interactions and their response to process variables such as time, temperature, pressure, pH, ionic strength, solubility, presence and concentration of excipients, agitation, and sequence of the process steps. Those skilled in the art, guided by the present invention and examples, would understand that these are the variables that would be identified to establish the procedural conditions needed to practice the inventions.

The invention is directed to dry, non-occlusive, pharmaceutical fibrous structures finished with an active pharmaceutical ingredient, or a pharmaceutically acceptable salt thereof. The invention is also directed to, among other things, methods of finishing a fibrous structure with an active pharmaceutical ingredient, as well as the finished fibrous structures produced according to the described methods.

These non-occlusive structures permit the passage of air, moisture vapor, and heat through the structure. In some aspects, the non-occlusive structures include a plurality of open pores that permit the passage of air, moisture vapor, and heat through the structure. The non-occlusive structures of the invention are in contrast to occlusive structures (e.g., structures having an oleophilic finish) which do not permit the passage of air, moisture vapor, and heat.

In preferred aspects, the dry, non-occlusive, pharmaceutical fibrous structures of the invention are hypoallergenic. In other aspects, the dry, non-occlusive, pharmaceutical fibrous structures are non-irritant. In other aspects, the dry, non-occlusive, pharmaceutical fibrous structures of the invention are substantially non-adhesive to a subject's skin. In some aspects, the non-adhesive structures are non-tacky at ambient temperature or above. In some aspects, the non-adhesive structures may be devoid of any adhesive material, i.e., the structures do not comprise any material that is tacky at ambient temperature or above. In other aspects, the non-adhesive structure may comprise a material that is tacky at ambient temperature and above, but the amount of that material is insufficient to confer tackiness to the structure at ambient temperature or above.

According to the invention, the dry, non-occlusive, pharmaceutical fibrous structures can be used to administer an active pharmaceutical ingredient to a subject. In these methods, the dry, non-occlusive, pharmaceutical fibrous structure is topically applied to the intact skin of the subject. “Intact skin” refers to skin in which there are no breaks, scrapes, cuts, or abnormal openings. According to the invention, the dry, non-occlusive, pharmaceutical fibrous structure is topically applied to the intact skin of the subject for a time sufficient to locally or systemically, or both, administer a therapeutically effective amount of the active pharmaceutical ingredient to the subject. In some aspects, the dry, non-occlusive, pharmaceutical fibrous structure is topically applied to the intact skin of the subject for a time sufficient to locally administer a therapeutically effective amount of the active pharmaceutical ingredient to the subject. In some aspects, the dry, non-occlusive, pharmaceutical fibrous structure is topically applied to the intact skin of the subject for a time sufficient to systemically administer a therapeutically effective amount of the active pharmaceutical ingredient to the subject. In some aspects, the dry, non-occlusive, pharmaceutical fibrous structure is topically applied to the intact skin of the subject for a time sufficient to locally and systemically administer a therapeutically effective amount of the active pharmaceutical ingredient to the subject.

In some aspects, the dry, non-occlusive, pharmaceutical fibrous structure is topically applied to the subject's intact skin for a time period of about 1 hour to about 168 hours. In some aspects, the dry, non-occlusive, pharmaceutical fibrous structure is topically applied to the subject's intact skin for a time period of about 1 hour to about 3 hours, 1 hour to about 6 hours, 1 hour to about 8 hours, 1 hour to about 12 hours, 1 hour to about 24 hours, 1 hour to about 48 hours, 1 hour to about 72 hours, 1 hour to about 96 hours, 1 hour to about 120 hours, 1 hour to about 144, or about 8 hours to about 72 hours. In some aspects, the dry, non-occlusive, pharmaceutical fibrous structure is topically applied to the subject's intact skin for a time period of about 24 hours to about 72 hours. In some aspects, the dry, non-occlusive, pharmaceutical fibrous structure is topically applied to the subject's intact skin for a time period of about 48 hours to about 72 hours. In some aspects, the dry, non-occlusive, pharmaceutical fibrous structure is topically applied to the subject's intact skin for a time period of about 4 days. In some aspects, the dry, non-occlusive, pharmaceutical fibrous structure is topically applied to the subject's intact skin for a time period of about 5 days. In some aspects, the dry, non-occlusive, pharmaceutical fibrous structure is topically applied to the subject's intact skin for a time period of about 6 days. In some aspects, the dry, non-occlusive, pharmaceutical fibrous structure is topically applied to the subject's intact skin for a time period of about 7 days.

In some aspects, the therapeutically effective amount of the active pharmaceutical ingredient is administered to the subject throughout the time period. That is, plasma levels sufficient to treat the subject's condition (either locally, systemically, or both) are substantially consistent throughout the time period. In some aspects, the therapeutically effective amount of the active pharmaceutical ingredient is administered to the subject for about 1 hour to about 72 hours. In some aspects, the therapeutically effective amount of the active pharmaceutical ingredient is administered to the subject for about 8 hours to about 72 hours. In some aspects, the therapeutically effective amount of the active pharmaceutical ingredient is administered to the subject for about 24 hours to about 72 hours. In some aspects, the therapeutically effective amount of the active pharmaceutical ingredient is administered to the subject for about 48 hours to about 72 hours. In some aspects, the therapeutically effective amount of the active pharmaceutical ingredient is administered to the subject for about 1, 2, 3, 4, 5, 6, 7, 8, 9, 10, 11, 12, 13, 14, 15, 16, 17, 18, 19, 20, 21, 22, 23, or about 24 hours. In some aspects, the therapeutically effective amount of the active pharmaceutical ingredient is administered to the subject for at least 4 hours. In some aspects, the therapeutically effective amount of the active pharmaceutical ingredient is administered to the subject for at least 6 hours. In some aspects, the therapeutically effective amount of the active pharmaceutical ingredient is administered to the subject for about 4 days. In some aspects, the therapeutically effective amount of the active pharmaceutical ingredient is administered to the subject for about 5 days. In some aspects, the therapeutically effective amount of the active pharmaceutical ingredient is administered to the subject for about 6 days. In some aspects, the therapeutically effective amount of the active pharmaceutical ingredient is administered to the subject for about 7 days.

The fibrous structures of the invention can comprise synthetic fibers, manmade fibers, and/or natural fibers, in any combination. Examples of synthetic fibers include, for example, polyamide fibers, acrylic fibers, elastane fibers, polyolefin fibers, polyester fibers and polylactic acid fibers. Examples of manmade fibers include, for example, modified cellulose fibers (e.g., rayon, lyocell), modified protein fibers (e.g., casein, soy, zein) and combinations thereof. Examples of natural fibers include, for example, protein fibers, cellulosic fibers (e.g., cotton, flax, hemp, jute), animal fibers (e.g., wool, mohair, cashmere), insect fibers (e.g., silk), and combinations thereof.

In some aspects, the fibrous structures comprise synthetic fibers. The structure may entirely comprise synthetic fibers, that is, 100% of the fibers are synthetic fibers. In other aspects, the fibrous structures comprise a blend of synthetic fibers and manmade fibers. In other aspects, the fibrous structures comprise a blend of synthetic fibers and natural fibers. In other aspects, the fibrous structures comprise a blend of synthetic fibers, manmade fibers, and natural fibers. In those aspects comprising a blend of synthetic fibers with other fibers, the fibrous structure may comprise about 5, 10, 15, 20, 25, 30, 35, 40, 45, 50, 55, 60, 65, 70, 75, 80, 85, 90, 91, 92, 93, 94, 95, 96, 97, 98, or about 99%, by weight, of synthetic fibers, with the remainder being manmade fibers, natural fibers, or a combination thereof.

In some aspects, the fibrous structures comprise manmade fibers. The structure may entirely comprise manmade fibers, that is, 100% of the fibers are manmade fibers. In other aspects, the fibrous structures comprise a blend of manmade and synthetic fibers. In other aspects, the fibrous structures comprise a blend of manmade fibers and natural fibers. In other aspects, the fibrous structures comprise a blend of manmade fibers, synthetic fibers, and natural fibers. In those aspects comprising a blend of manmade fibers with other fibers, the fibrous structure may comprise about 5, 10, 15, 20, 25, 30, 35, 40, 45, 50, 55, 60, 65, 70, 75, 80, 85, 90, 91, 92, 93, 94, 95, 96, 97, 98, or about 99%, by weight, of manmade fibers, with the remainder being synthetic fibers, natural fibers, or a combination thereof.

In some aspects, the fibrous structures comprise natural fibers. The structure may entirely comprise natural fibers, that is, 100% of the fibers are natural fibers. In other aspects, the fibrous structures comprise a blend of natural fibers and manmade fibers. In other aspects, the fibrous structures comprise a blend of natural fibers and synthetic fibers. In other aspects, the fibrous structures comprise a blend of natural fibers, synthetic fibers, and manmade fibers. In those aspects comprising a blend of natural fibers with other fibers, the fibrous structure may comprise about 5, 10, 15, 20, 25, 30, 35, 40, 45, 50, 55, 60, 65, 70, 75, 80, 85, 90, 91, 92, 93, 94, 95, 96, 97, 98, or about 99%, by weight, of natural fibers, with the remainder being synthetic fibers, manmade fibers, or a combination thereof.

The fibrous structures of the invention can be in any shape or form. For example, fibrous structures of the invention can be in the form of sheets or tubes. As used herein, the term “denier” refers to the linear mass density of a filament expressed as the mass in grams per 9000 meters of filament. The denier per filament (DPF) of the constituent fibers can range from 0.1 to 100 denier. The yarns may be made of staple fibers or continuous fibers with yarn denier ranging from 1 to 10,000 denier, plied or single, twisted or untwisted, textured or flat. The fabrics may be woven fabrics in plain weave, twill, sateen or jacquard interlacing. The fabrics may be knit fabrics either weft or warp knit, flat knit or circular knit, seamed or seamless. The fabrics may be nonwoven fabrics produced on needle punched, wet laid, air laid, thermobonded, calendared, spun-laced, spun-bonded, melt-blown or electrospun systems. The fabrics may be braided using any number of interlaced strands. In other aspects, the fibrous structures are in the form of a wearable garment, in particular a garment that can remain in contact with the skin on any place of the body or face. In certain aspects, the wearable garment fits tightly or snugly against the skin or body part of the wearer.

The fibrous structures of the invention may optionally be scoured, bleached, or finished using conventional techniques. Alternatively, fibrous structures of the invention may be dyed or printed any color, using techniques known in the art.

Some aspects of the invention are directed to finished, dry, non-occlusive, pharmaceutical fibrous structures that have been finished with a finishing composition comprising an active pharmaceutical ingredient, or a pharmaceutically acceptable salt thereof, a surfactant and a solvent. While not wishing to be bound by any particular theory, it is believed that the addition of a surfactant may facilitate dispersion of the API (or salt thereof) in the finishing composition. The finishing composition may optionally also comprise a humectant. While not wishing to be bound by any particular theory, it is believed that the addition of a humectant may improve coating flexibility by holding on to water. The finishing composition may optionally also comprise a permeation enhancer. While not wishing to be bound by any particular theory, it is believed that the addition of a permeation enhancer may improve flow during deposition during the finishing treatment, improving uniformity (e.g., leveling) and/or increase permeation of the API through the subject's skin. In some aspects, the finishing composition may also comprise a humectant, a permeation enhancer, or a combination thereof.

In preferred aspects, the finished, dry, non-occlusive, pharmaceutical fibrous structures of the invention that have been finished with finishing compositions, are hypoallergenic. In other aspects, these finished, dry, non-occlusive, pharmaceutical fibrous structures are non-irritant. In other aspects, these finished, dry, non-occlusive, pharmaceutical fibrous structures of the invention are substantially non-adhesive to a subject's skin. In some aspects, these non-adhesive structures are non-tacky at ambient temperature or above. In some aspects, these non-adhesive structures may be devoid of any adhesive material, i.e., the structures do not comprise any material that is tacky at ambient temperature or above. In other aspects, these non-adhesive structure may comprise a material that is tacky at ambient temperature and above, but the amount of that material is insufficient to confer tackiness to the structure at ambient temperature or above.

The fibrous structures of the finished, dry, non-occlusive, pharmaceutical fibrous structures of the invention can comprise synthetic fibers, manmade fibers, and/or natural fibers as described herein. For example, in some of these aspects, the fibrous structures comprise synthetic fibers as described herein. In other aspects, the fibrous structures comprise manmade fibers. In other aspects, the fibrous structures comprise natural fibers as described herein. In other aspects, the fibrous structures comprise a blend of synthetic fibers and manmade fibers as described herein. In other aspects, the fibrous structures comprise a blend of synthetic fibers and natural fibers as described herein. In other aspects, the fibrous structures comprise a blend of manmade fibers and natural fibers as described herein. In other aspects, the fibrous structures comprise a blend of synthetic fibers, manmade fibers, and natural fibers as described herein.

The active pharmaceutical ingredients (APIs), or pharmaceutically acceptable salts thereof, can be present in the finishing compositions of the invention in an amount of from about 0.1% (w/w) to about 25% (w/w). For example, the API or API salt can be present in the finishing composition in an amount of about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, or about 25% (w/w). In some aspects, the API or API salt can be present in the finishing composition in an amount of from about 0.1% (w/w) to about 15% (w/w) or from about 0.1% (w/w) to about 10% (w/w). In some aspects, the API or API salt can be present in the finishing composition in an amount of from about 1% (w/w) to about 10% (w/w) or from about 10% (w/w) to about 25% (w/w). In some aspects, the API or API salt can be present in the finishing composition in an amount of from about 15% (w/w) to about 25% (w/w).

APIs useful in all aspects of the invention are those known in the art to be useful for transdermal or dermal administration to a subject. Those APIs that are particularly envisioned for use with the claimed methods and fibrous structures are those APIs that can be administered topically (transdermally or dermally) and that achieve a local-acting effect with low levels of systemic exposure. In other aspects, the APIs can be administered topically and achieve therapeutically effective systemic levels of exposure in the subject. In other aspects, the APIs can be administered topically and achieve therapeutically effective systemic levels of exposure in the subject, as well as therapeutically effective local levels of exposure in the subject. Preferred APIs are generally lipophilic. Alternatively, the API can be hydrophilic.

The present invention relates to a method of treating a condition, comprising applying to the intact skin of a body part of a subject in need thereof, an item of clothing, wherein said item of clothing comprises a dry, non-occlusive, fibrous structure, wherein said item of clothing has been finished with a finishing composition that is a homogeneous dispersion comprising an active pharmaceutical ingredient, a surfactant and a solvent, wherein said condition comprises either a painful condition, an inflammatory condition, an infective condition, or a dermatological condition and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

As used herein, the term ‘item of clothing’ shall refer to a sock, a knee brace, a shoulder brace, an elbow sleeve, a wrist band, a glove, a neck sleeve, a back brace, a sleeve, a t-shirt or a bandage.

As used herein, the term ‘sock’ shall refer to an item of clothing designed for wearing over the foot. In one embodiment, the sock is designed for wearing over the foot and ankle (ankle sock). In another embodiment, the sock is designed for wearing over the foot, ankle and shin (mid-length sock). In another embodiment, the sock is designed for wearing over the foot, ankle, shin and knee (knee-length sock). In another embodiment, the sock is designed for wearing over the foot, ankle, shin, knee and lower thigh (high-length sock). In one embodiment, the sock is designed to be worn with each toe individually encased by the sock (toed sock). In another embodiment, the sock is designed to worn over all toes together (non-toed sock). In another embodiment, the sock is designed to worn with no toes encased within it (toeless sock). It is to be understood that both toed socks, toeless, and non-toed socks can be any of ankle socks, mid-length socks, knee-length socks and high-length socks.

As used herein, the term ‘knee brace’ shall refer to an item of clothing designed for wearing over the knee and which does not extend to also being worn over the foot or upper thigh.

As used herein, the term ‘shoulder brace’ shall refer to an item of clothing designed for wearing over the shoulder, outer third of the adjacent clavicle and upper third of the adjacent arm.

As used herein, the term ‘elbow sleeve’ shall refer to an item of clothing designed for wearing over the elbow and which does not extend to also being worn over the hand.

As used herein, the term ‘wrist band’ shall refer to an item of clothing designed for wearing over the wrist and which does not extend to also being worn over the hand or fingers.

As used herein, the term ‘glove’ shall refer to an item of clothing designed for wearing over the hand and/or fingers. In one embodiment, the glove is designed for wearing over the wrist, hand and fingers (short glove). In another embodiment, the glove is designed for wearing over the elbow, wrist, hand, and fingers (elbow glove). In one embodiment, the glove is designed to be worn with each finger individually encased within the glove (finger glove). In another embodiment, the glove is designed to be worn with no finger encased within it (fingerless glove). In yet another embodiment, the glove is designed to be worn with all the fingers, but not necessarily the thumb, encased together within the glove (mitten). It is to be understood that finger gloves, fingerless gloves and mittens can be either short gloves or elbow gloves.

As used herein, the term ‘neck sleeve’ shall refer to an item of clothing designed for wearing around the neck. In one embodiment, the neck sleeve is a continuous loop of clothing (closed neck sleeve). In another embodiment, the neck sleeve is an open item of clothing for wrapping around the neck (scarf).

As used herein, the term ‘back brace’ shall refer to an item of clothing designed for wearing around the lower torso and which does not extend to the chest.

As used herein, the term ‘sleeve’ shall refer to a tubular item of clothing designed for wearing over a body appendage, such as a finger, leg or arm and which is open at, at least one, end.

As used herein, the term ‘t-shirt’ shall refer to an item of clothing designed for wearing over the torso. In one embodiment, the t-shirt is designed for wearing only over the shoulders and chest and does not extend down to the abdomen (short-shirt). In another embodiment, the t-shirt is designed for wearing over the shoulders, chest and abdomen (long-shirt).

As used herein, the term ‘bandage’ shall refer to a non-occlusive item of clothing designed for wrapping around the intact skin of the body part in need thereof.

As used herein, the term “active pharmaceutical ingredient” shall refer to the drug and its pharmaceutically acceptable salts and/or esters thereof.

As used herein, the term ‘painful condition’ shall refer to a condition that primarily manifests to the subject as pain. In one embodiment, the painful condition is nociceptive pain. In another embodiment, the painful condition is neuropathic pain.

Nociceptive pain is pain associated with stimulation of sensory nerve fibers and can be either superficial, deep or a mixture of the two.

Examples of superficial nociceptive pain include the pain associated with abrasions, cuts, bruises, burns, sunburns or insect bites.

Examples of deep nociceptive pain include musculoskeletal, post-operative and inflammatory response related pain. Examples of musculoskeletal pain include the pain associated with sprains, tears to ligaments, tendons or muscle tissue, bone fractures, inflammation of tendons, joints, bursae or fascia and degenerative changes to the musculoskeletal system.

Neuropathic pain is pain associated with damage to, or disease affecting, the nervous system and can be either central, peripheral or mixed central and peripheral.

Central neuropathic pain is pain predominantly associated with a lesion to the central nervous system and can be either spinal or supra-spinal in origin. Examples of supra-spinal central neuropathic pain include the pain associated with a central venous thrombosis, a cerebral tumor, a traumatic brain lesion, multiple sclerosis, Parkinson's disease and the pain occurring after a stroke or a thalamotomy. Examples of spinal central neuropathic pain include the pain associated with a spinal cord injury, complications of spinal surgery, ischemic lesions of the spinal cord, radiation or HIV associated myelopathy.

Peripheral neuropathic pain is pain predominantly associated with the peripheral nervous system and includes the pain associated with lesions and compressions to the peripheral nervous system. Examples of peripheral neuropathic pain associated with lesions to the peripheral nervous system includes the pain associated with lesions to peripheral nerves, lesions to nerve roots and posterior ganglia and lesions to cranial nerves. Examples of peripheral neuropathic pain associated with compressions to the peripheral nervous system include the pain associated with compressions to peripheral nerves, nerve plexuses and nerve roots. Examples of peripheral neuropathic pain associated with lesions to peripheral nerves include the pain associated with length dependent neuropathies, those lesions caused by systemic disease such as diabetic neuropathy, lupus neuropathy or hypothyroidism associated neuropathy, drug-induced disorders such as chemotherapy induced neuropathy, metabolic or nutritional disorders such as alcoholic neuropathy, traumatic and entrapment syndromes, post-surgical nerve injuries, inflammatory demyelinating polyradiuloneuropathy and HIV sensory neuropathy. Examples of peripheral neuropathic pain associated with lesions to nerve roots and posterior ganglia include the pain associated with those lesions associated with postherpetic neuralgia and nerve root avulsions. Examples of peripheral neuropathic pain associated with lesions to cranial nerves include the pain associated with cranial neuralgias such as trigeminal neuralgia. Other examples of peripheral neuropathic pain include the pain associated with paraneoplastic peripheral neuropathy and ganglionopathy, complications of chemotherapy, radiotherapy and surgery and both Type 1 and Type 2 Complex Regional Pain Syndromes.

Therapeutically effective active pharmaceutical ingredients for treating pain are either generally effective in treating pain or are specific to the pathophysiology causing the pain. Examples of active pharmaceutical ingredients generally effective in treating pain include one or more of the following: local anesthetics, non-steroidal anti-inflammatory drugs (NSAIDS), anticonvulsants and other topical analgesic agents. Examples of local anesthetics include one or more of the following: articaine, benzocaine, bupivacaine, butamben, cinchocaine, chloroprocaine, cyclomethycaine, dibucaine, dimethisoquin, dyclonine, etidocaine, levobupivacaine, lidocaine, mepivacaine, pramoxine, piperocaine, prilocaine, propoxycaine, procaine, proparacaine, ropivacaine, tetracaine and trimecaine. Examples of NSAIDs include one or more of the following: aceclofenac, aspirin, benzydamine, celecoxib, dexketoprofen, diflunisal, droxicam, dexibuprofen, diclofenac, enolic acid, etodolac, etoricoxib, fenoprofen, firocoxib, flufenamic acid, flurbiprofen, ibuprofen, indomethacin, ketoprofen, ketorolac, lornoxicam, loxoprofen, meclofenamic acid, mefenamic acid, meloxicam, nabumetone, naproxen, nimesulide, oxaprozin, paracetamol, parecoxib, phenylbutazone, piroxicam, salsalate, sulindac, tenoxicam, tolmetin and tolfenamic acid. Examples of anticonvulsants include one or more of the following: one or more of the following: carbamazepine, gabapentin, lacosamide, lamotrigine, levetiracetam, oxcarbazepine, pregabalin, tiagabine, valproate and vigabatrin. Examples of other topical analgesic drugs include one or more of the following: capsaicin, menthol, clonidine, detomidine, dexmedetomidine, duloxetine, medetomidine, brimonidine, guanabenz and camphor.

As used herein, the term ‘inflammatory condition’ shall refer to shall refer to a condition that primarily manifests to the subject as inflammation. In one embodiment, the inflammatory condition is a locally manifesting inflammatory condition. In another embodiment, the inflammatory condition is a systemic inflammatory condition.

A locally manifesting inflammatory condition is a condition caused by an inflammation generally to the skin, muscle and/or surrounding tissues without significant additional systemic involvement.

Examples of locally manifesting inflammatory conditions include bursitis, dermatomyositis, lichen planus, neuritis, panniculitis, phlebitis, tendonitis and tendinosis.

A systemic inflammatory condition is a condition caused by an inflammation, which affects multiple body organs including, but not limited to, the skin, muscle and/or surrounding tissues.

Examples of systemic inflammatory conditions include autoimmune diseases, gout, inflammatory bowel disease, pseudogout, rheumatic fever, sarcoidosis and vasculitis. Examples of autoimmune diseases include ankylosing spondylitis, juvenile idiopathic arthritis (JIA), mast cell activation syndrome (MCAS), psoriasis, reactive arthritis, rheumatoid arthritis, Sjögren's syndrome and systemic lupus erythematosus (SLE).

Therapeutically effective active pharmaceutical ingredients for treating inflammation are either generally effective in treating inflammation or are specific to the pathophysiology causing the inflammation. Examples of active pharmaceutical ingredients generally effective in treating inflammation include one or more of the following: anti-histamines, anti-leukotrienes, anti-metabolites, corticosteroids, disease-modifying antirheumatic drugs (DMARDs), immunomodulators, immunosuppressants and non-steroidal anti-inflammatory drugs (NSAIDS). Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine. Examples of anti-leukotrienes include one or more of the following: curcumin, montelukast, pranlukast, St. John's wort, zafirlukast and zileuton. Examples of anti-metabolites include one or more of the following: azathioprine, leflunomide, mercaptopurine, methotrexate, mycophenolic acid, pemetrexed and pralatrexate. Examples of corticosteroids include one or more of the following: alclometasone, amcinonide, beclometasone, betamethasone, budesonide, ciclesonide, clobetasol, clobetasone, clocortolone, cortisone, desonide, desoximetasone, dexamethasone, diflorasone, fluocinolone, fluocortolone, fluprednidene, flurandrenolide, fluticasone, halcinonide, halobetasol, halometasone, hydrocortisone, mometasone, methylprednisolone, prednicarbate, prednisolone, prednisone, tixocortol, triamcinolone, mometasone. Examples of immunomodulators include one or more of the following: apremilast, azathioprine, lenalidomide, methotrexate, pentoxifylline, pomalidomide and thalidomide. Examples of DMARDs include one or more of the following: azathioprine, chloroquine, ciclosporin, gold salts, hydroxychloroquine, leflunomide, mesalazine, methotrexate, sulfasalazine and tofacitinib. Examples of immunosuppressants include one or more of the following: azathioprine, cyclophosphamide, cladribine, chlorambucil, cyclophosphamide, cyclosporine, everolimus, mercaptopurine, methotrexate, mycophenolic acid, sirolimus and tacrolimus. Examples of NSAIDs include one or more of the following: aceclofenac, aspirin, benzydamine, celecoxib, dexketoprofen, diflunisal, droxicam, dexibuprofen, diclofenac, enolic acid, etodolac, etoricoxib, fenoprofen, firocoxib, flufenamic acid, flurbiprofen, ibuprofen, indomethacin, ketoprofen, ketorolac, lornoxicam, loxoprofen, meclofenamic acid, mefenamic acid, meloxicam, nabumetone, naproxen, nimesulide, oxaprozin, paracetamol, parecoxib, phenylbutazone, piroxicam, salsalate, sulindac, tenoxicam, tolmetin and tolfenamic acid.

As used herein, the term ‘infective condition’ shall refer to shall refer to a condition that manifests to the subject as an infection of the skin.

In one embodiment of the invention, the infective condition is a mycotic infection. Examples of mycotic infections include tinea versicolor, tinea corporis, tinea pedis and onychomycosis. In another embodiment of the invention, the infective condition is a bacterial infection. Examples of bacterial infections include folliculitis, impetigo, erysipelas, cellulitis and infections associated with minor cuts, scrapes or burns. In another embodiment of the invention, the infective condition is a parasitic infection. Examples of parasitic infection include scabies, leishmaniasis and hookworm infections. In another embodiment of the invention, the infective condition is a viral infection. Examples of viral infections include those associated with varicella zoster virus and human papillomavirus.

Therapeutically effective active pharmaceutical ingredients for treating infections are typically specific to the agent causing the infection. Examples of active pharmaceutical ingredients effective in treating folliculitis include one or more of the following: bacitracin, benzoyl peroxide, cephalexin, ciprofloxacin, clindamycin, dapsone, erythromycin, loxacillin, dicloxacillin, flucloxacillin, linezolid, minocycline, mupirocin, neomycin, polymyxin B, rifampin and trimethoprim-sulfamethoxazole. Examples of active pharmaceutical ingredients effective in treating impetigo include one or more of the following: amoxicillin-clavulanate potassium, bacitracin, cephalexin, chloramphenicol, ciprofloxacin, clindamycin, doxycycline, erythromycin, fusidic acid, gentamicin, levofloxacin, linezolid, mupirocin, neosporin, ofloxacin, ozenoxacin, retapamulin, tetracycline and trimethoprim-sulphamethoxazole. Examples of active pharmaceutical ingredients effective in treating erysipelas include one or more of the following: ampicillin, amoxicillin, amoxicillin-clavulanate potassium, azithromycin, azlocillin, bacampicillin, carbenicillin, cephalexin, clindamycin, cloxacillin, dicloxacillin, epicillin, erythromycin, flucloxacillin, hetacillin, metampicillin, methicillin, mezlocillin, nafcillin, penicillin, piperacillin, pivampicillin, oxacillin, sulbactam, talampicillin, tazobactam, temocillin and ticarcillin. Examples of active pharmaceutical ingredients effective in treating cellulitis include one or more of the following: amoxicillin, ampicillin-sulbactam, azithromycin, cefuroxime, cephalexin, ceftriaxone, cefazolin, clarithromycin, clindamycin, dicloxacillin, erythromycin, levofloxacin, nafcillin, oxacillin, penicillin and vancomycin. Examples of active pharmaceutical ingredients effective in treating infections associated with minor cuts, scrapes or burns include one or more of the following: bacitracin, chlortetracycline, neomycin and tetracycline. Examples of active pharmaceutical ingredients effective in treating tinea versicolor include one or more of the following: butenafine, selenium and terbinafine. Examples of active pharmaceutical ingredients effective in treating tinea corporis include one or more of the following: betamethasone, butenafine, ciclopirox, clioquinol, clotrimazole, econazole, haloprogin, luliconazole, miconazole, naftifine, oxiconazole, povidone-iodine, sertaconazole, sulconazole, terbinafine, tolnaftate and undecylenic acid. Examples of active pharmaceutical ingredients effective in treating tinea pedis include one or more of the following: betamethasone, butenafine, ciclopirox, clioquinol, clotrimazole, econazole, haloprogin, luliconazole, miconazole, naftifine, oxiconazole, povidone-iodine, sertaconazole, sulconazole, terbinafine, tolnaftate and undecylenic acid. Examples of active pharmaceutical ingredients effective in treating scabies include one or more of the following: benzyl benzoate, crotamiton, ivermectin, lindane, moxidectin, permethrin, precipitated sulfur, salicylic acid and synergized pyrethrins. Examples of active pharmaceutical ingredients effective in treating leishmaniasis include one or more of the following: allopurinol, amphotericin B, antimonite, cotrimoxazole, dapsone, fluconazole, ketoconazole, meglumine, methylbenzethonium chloride, metronidazole, miltefosine, paromomycin, pentoxifylline, raconazole, rifampicin and sodium stibogluconante. Examples of active pharmaceutical ingredients effective in treating hookworm infections include one or more of the following: albendazole, levamisole, mebendazole and pyrantel pamoate. Examples of active pharmaceutical ingredients effective in treating varicella zoster virus infections include one or more of the following: acyclovir, famciclovir, penciclovir and valacyclovir. Examples of active pharmaceutical ingredients effective in treating human papillomavirus infections include one or more of the following: benzoyl peroxide, bleomycin, cantharidin, dinitrochlorobenzene, formic acid, imiquimod, podophyllin, salicylic acid, silver nitrate, trichloroacetic acid, zinc oxide and zinc sulfate.

As used herein, the term ‘dermatological condition’ shall refer to shall refer to a condition that primarily manifests on the subject's skin or dermal tissues. In one embodiment of the invention, the dermatological condition is an allergic condition. In another embodiment of the invention, the dermatological condition is a cutaneous malignancy. In another embodiment of the invention, the dermatological condition is a blister. In another embodiment of the invention, the dermatological condition is pruritis. In another embodiment of the invention, the dermatological condition is an acneiform eruption. In another embodiment of the invention, the dermatological condition is a keratosis. In another embodiment of the invention, the dermatological condition is a cutaneous condition secondary to an external cause. In another embodiment of the invention, the dermatological condition is a corticosteroid-responsive dermatoses.

Examples of allergic conditions include atopic dermatitis, contact dermatitis and urticaria. Examples of cutaneous malignancies include cutaneous t-cell lymphomas, superficial basal cell carcinomas and AIDS-related Kaposi's sarcoma. Examples of blisters include dermatitis herpetiformis, pemphigus and pemphigoid. Examples of keratosis include actinic keratosis and hyperkeratosis.

Therapeutically effective active pharmaceutical ingredients for treating dermatological condition are either generally effective in treating dermatological conditions or are specific to the pathophysiology involved. Examples of active pharmaceutical ingredients generally effective in treating dermatological conditions include one or more of the following: anti-histamines, anti-leukotrienes, corticosteroids, immunomodulators, immunosuppressants, local anesthetics and topical analgesic drugs. Examples of anti-histamines include one or more of the following: acrivastine, azelastine, bepotastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, ketotifen, levocabastine, levocetirizine, loratadine, meclizine, mirtazapine, mizolastine, olopatadine, orphenadrine, phenindamine, pheniramine, phenyltoloxamine, promethazine, pyrilamine, rupatadine, tripelennamine and triprolidine. Examples of anti-leukotrienes include one or more of the following: curcumin, montelukast, pranlukast, St. John's wort, zafirlukast and zileuton. Examples of corticosteroids include one or more of the following: alclometasone, amcinonide, beclometasone, betamethasone, budesonide, ciclesonide, clobetasol, clobetasone, clocortolone, cortisone, desonide, desoximetasone, dexamethasone, diflorasone, fluocinolone, fluocortolone, fluprednidene, flurandrenolide, fluticasone, halcinonide, halobetasol, halometasone, hydrocortisone, mometasone, methylprednisolone, prednicarbate, prednisolone, prednisone, tixocortol, triamcinolone, mometasone. Examples of immunomodulators include one or more of the following: apremilast, azathioprine, lenalidomide, methotrexate, pentoxifylline, pomalidomide and thalidomide. Examples of immunosuppressants include one or more of the following: azathioprine, cyclophosphamide, cladribine, chlorambucil, cyclophosphamide, cyclosporine, everolimus, mercaptopurine, methotrexate, mycophenolic acid, pimecrolimus, sirolimus and tacrolimus. Examples of local anesthetics include one or more of the following: articaine, benzocaine, bupivacaine, butamben, cinchocaine, chloroprocaine, cyclomethycaine, dibucaine, dimethisoquin, dyclonine, etidocaine, levobupivacaine, lidocaine, mepivacaine, pramoxine, piperocaine, prilocaine, propoxycaine, procaine, proparacaine, ropivacaine, tetracaine and trimecaine. Examples of topical analgesic drugs include one or more of the following: capsaicin, menthol, clonidine, detomidine, dexmedetomidine, duloxetine, medetomidine, brimonidine, guanabenz and camphor.

It is generally understood that inflammatory conditions can also manifest with pain, infection, and even dermatological presentations. It is also to be generally understood that infective conditions can also manifest with pain, inflammation, and even dermatological presentations and that dermatological presentations can also manifest with pain, inflammation, and even infection.

The present application also relates to a method of treating a painful condition of the feet, comprising applying to the intact skin of a foot of a subject in need thereof, a sock, and wherein said sock comprises a dry, non-occlusive, fibrous structure, wherein said sock has been finished with a finishing composition that is a homogeneous dispersion comprising an active pharmaceutical ingredient, a surfactant and a solvent, wherein said painful condition comprises either a superficial nociceptive pain, a deep nociceptive pain, a supra-spinal central neuropathic pain, a spinal central neuropathic pain, a peripheral neuropathic pain or a mixed central and peripheral neuropathic pain and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of superficial nociceptive pain of the feet include the pain associated with abrasions, cuts, bruises, burns, sunburns or insect bites in or around the foot.

Examples of deep nociceptive pain of the feet include the pain associated with musculoskeletal, post-operative and inflammatory response related pain in or around the foot. Examples of musculoskeletal pain of the feet include the pain associated with sprains, tears to ligaments, tendons or muscle tissue, bone fractures, inflammation of tendons, joints, bursae or fascia and degenerative changes to the musculoskeletal system in or around the foot. Examples of the pain associated with sprains of the feet include the pain associated with ankle sprains and metatarsalgia. Examples of the pain associated with inflammation of tendons of the feet include the pain associated with Achilles tendinitis or Achilles tendinosis. Examples of the pain associated with inflammation of joints of the feet include the pain associated with hallux valgus, bunions, bunionettes (tailor's bunions). Examples of the pain associated with inflammation of bursae of the feet include the pain associated with Achilles bursitis. Examples of the pain associated with inflammation of fascia of the feet include the pain associated with plantar fasciitis. Examples of the pain associated with degenerative changes to the musculoskeletal system of the feet include the pain associated with arthritis of the ankle or foot.

Examples of central neuropathic pain of the feet include the pain associated with spinal or supra-spinal lesions to the central nervous system which manifest in or around the foot. Examples of the pain of the feet associated with supra-spinal lesions to the central nervous system include the pain associated with central venous thrombosis, with a cerebral tumor, with a traumatic brain lesion, with multiple sclerosis, with Parkinson's disease and the pain occurring after a stroke or a thalamotomy, as presenting on or around the foot. Examples of the pain of the feet associated with spinal lesions to the central nervous system include the pain associated with, with a spinal cord injury, with complications of spinal surgery, with ischemic lesions of the spinal cord, with radiation or HIV associated myelopathy, as presenting on or around the foot.

Examples of peripheral neuropathic pain of the feet include the pain associated with lesions and compressions to the peripheral nervous system in or around the foot. Examples of the pain associated with lesions to peripheral nerves of the feet include the pain associated with length dependent neuropathy, diabetic neuropathy, lupus neuropathy, Sjogren's syndrome associated neuropathy, hypothyroidism associated neuropathy, HIV related neuropathy, alcoholic neuropathy and inflammatory demyelinating polyradiuloneuropathy, in or around the foot. Examples of the pain associated with compressions to peripheral nerves, nerve plexuses and nerve roots of the feet include Morton's interdigital neuroma, tarsal tunnel syndrome and entrapment syndromes including those of the interdigital nerves, the first branch of the lateral plantar nerve, the isolated medial or lateral plantar nerves, the posterior tibial nerve, the deep peroneal nerve, the superficial peroneal nerve, the sural nerve and the saphenous nerve.

Examples of the pain associated with lesions to nerve roots and posterior ganglia which manifest in or around the foot include the pain associated with postherpetic neuralgia and nerve root avulsions, as presenting in or around the foot. Other examples of peripheral neuropathic pain of the feet include the pain associated with paraneoplastic peripheral neuropathy and ganglionopathy, with complications of chemotherapy, radiotherapy and surgery and both Type 1 and Type 2 Complex Regional Pain Syndromes, as presenting in or around the foot.

In one embodiment of the invention, the method of treating the pain associated with abrasions, cuts, bruises, burns, sunburns and insect bites of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the feet comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating deep nociceptive pain of the foot comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the foot comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with an ankle sprain comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with an ankle sprain comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with an ankle sprain comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with an ankle sprain comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with an ankle sprain comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with an ankle sprain comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with an ankle sprain comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with an ankle sprain comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with an ankle sprain comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with metatarsalgia comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with metatarsalgia comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with metatarsalgia comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with metatarsalgia comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with metatarsalgia comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with metatarsalgia comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles tendinitis comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles tendinitis comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles tendinitis comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles tendinitis comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles tendinitis comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles tendinitis comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles tendinitis comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles tendinitis comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles tendinitis comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles tendinosis comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles tendinosis comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles tendinosis comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles tendinosis comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles tendinosis comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles tendinosis comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles tendinosis comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles tendinosis comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles tendinosis comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with hallux valgus comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with hallux valgus comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with hallux valgus comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with bunions comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with bunions comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with bunions comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with bunions comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with bunions comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with bunionettes comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with bunionettes comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with bunionettes comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles bursitis comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles bursitis comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles bursitis comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles bursitis comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles bursitis comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles bursitis comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles bursitis comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles bursitis comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Achilles bursitis comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with plantar fasciitis comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with plantar fasciitis comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with plantar fasciitis comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with plantar fasciitis comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with plantar fasciitis comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with plantar fasciitis comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the ankle comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the ankle comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the ankle comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the ankle comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the ankle comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the ankle comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the ankle comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a local anesthetic. In another preferred embodiment, the active pharmaceutical ingredient is an NSAID In yet another preferred embodiment, the active pharmaceutical ingredient is a topical analgesic drug.

In one embodiment of the invention, the method of treating the pain associated with arthritis of the ankle comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the ankle comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the foot comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the foot comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the foot comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the foot comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the foot comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the foot comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the foot comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID In yet another preferred embodiment, the active pharmaceutical ingredient is a topical analgesic drug.

In one embodiment of the invention, the method of treating the pain associated with arthritis of the foot comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the foot comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the feet associated with supra-spinal lesions to the central nervous system comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with supra-spinal lesions to the central nervous system comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a sock, wherein said sock comprises a dry, non-occlusive, fibrous structure, and wherein said sock has been finished with a finishing composition that is a and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the feet associated with spinal lesions to the central nervous system comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating peripheral neuropathic pain of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the feet comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating mixed central and peripheral neuropathic pain of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone

In one embodiment of the invention, the method of treating mixed central and peripheral neuropathic pain of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the feet comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating the pain associated with length dependent neuropathies of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the feet comprises applying to the intact skin of a foot of a subject in need thereof, an ankle sock, wherein said sock comprises a dry, non-occlusive, fibrous structure, and wherein said sock has been finished with a finishing composition that is a and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the feet comprises applying to the intact skin of a foot of a subject in need thereof, a knee-length sock, wherein said sock comprises a dry, non-occlusive, fibrous structure, and wherein said sock has been finished with a finishing composition that is a and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Sjogren's syndrome associated neuropathies of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Sjogren's syndrome associated neuropathies of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Sjogren's syndrome associated neuropathies of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Sjogren's syndrome associated neuropathies of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Sjogren's syndrome associated neuropathies of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Sjogren's syndrome associated neuropathies of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Sjogren's syndrome associated neuropathies of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Sjogren's syndrome associated neuropathies of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Sjogren's syndrome associated neuropathies of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Sjogren's syndrome associated neuropathies of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Sjogren's syndrome associated neuropathies of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Sjogren's syndrome associated neuropathies of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Sjogren's syndrome associated neuropathies of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Sjogren's syndrome associated neuropathies of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Sjogren's syndrome associated neuropathies of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Sjogren's syndrome associated neuropathies of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with alcoholic neuropathies of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with alcoholic neuropathies of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with alcoholic neuropathies of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with alcoholic neuropathies of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with alcoholic neuropathies of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with alcoholic neuropathies of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with alcoholic neuropathies of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with alcoholic neuropathies of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with alcoholic neuropathies of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with alcoholic neuropathies of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with alcoholic neuropathies of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with alcoholic neuropathies of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with alcoholic neuropathies of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with alcoholic neuropathies of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with alcoholic neuropathies of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with alcoholic neuropathies of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with chemotherapy as manifesting in or around the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with chemotherapy as manifesting in or around the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with chemotherapy as manifesting in or around the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with chemotherapy as manifesting in or around the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with chemotherapy as manifesting in or around the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with chemotherapy as manifesting in or around the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with chemotherapy as manifesting in or around the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with chemotherapy as manifesting in or around the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with chemotherapy as manifesting in or around the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with chemotherapy as manifesting in or around the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with chemotherapy as manifesting in or around the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with chemotherapy as manifesting in or around the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Morton's interdigital neuroma comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Morton's interdigital neuroma comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Morton's interdigital neuroma comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Morton's interdigital neuroma comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Morton's interdigital neuroma comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Morton's interdigital neuroma comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with tarsal tunnel syndrome comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with tarsal tunnel syndrome comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with tarsal tunnel syndrome comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with tarsal tunnel syndrome comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the interdigital nerves of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the interdigital nerves of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the interdigital nerves of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the interdigital nerves of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the interdigital nerves of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the interdigital nerves of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the lateral plantar nerve comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the lateral plantar nerve comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the lateral plantar nerve comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the lateral plantar nerve comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the lateral plantar nerve comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the lateral plantar nerve comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the medial plantar nerve comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the medial plantar nerve comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the medial plantar nerve comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the medial plantar nerve comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the medial plantar nerve comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the medial plantar nerve comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the posterior tibial nerve comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the posterior tibial nerve comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the posterior tibial nerve comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the posterior tibial nerve comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the posterior tibial nerve comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the posterior tibial nerve comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the posterior tibial nerve comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the posterior tibial nerve comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the posterior tibial nerve comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the posterior tibial nerve comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the deep peroneal nerve comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the deep peroneal nerve comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the deep peroneal nerve comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the deep peroneal nerve comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the deep peroneal nerve comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the deep peroneal nerve comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the deep peroneal nerve comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the deep peroneal nerve comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the deep peroneal nerve comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the deep peroneal nerve comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the deep peroneal nerve comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the deep peroneal nerve comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug

In one embodiment of the invention, the method of treating the pain associated with entrapment syndromes of the superficial peroneal nerve comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the superficial peroneal nerve comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the superficial peroneal nerve comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the superficial peroneal nerve comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the superficial peroneal nerve comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the superficial peroneal nerve comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the superficial peroneal nerve comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the superficial peroneal nerve comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the superficial peroneal nerve comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the superficial peroneal nerve comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the superficial peroneal nerve comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the superficial peroneal nerve comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the sural nerve comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the sural nerve comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the sural nerve comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the sural nerve comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the sural nerve comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the sural nerve comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the sural nerve comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the sural nerve comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the sural nerve comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the saphenous nerve comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the saphenous nerve comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the saphenous nerve comprises applying to the intact skin of a foot of a subject in need thereof, an ankle sock, wherein said sock comprises a dry, non-occlusive, fibrous structure, and wherein said sock has been finished with a finishing composition that is a and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the saphenous nerve comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the saphenous nerve comprises applying to the intact skin of a foot of a subject in need thereof, a mid-length ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the saphenous nerve comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the saphenous nerve comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the saphenous nerve comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the saphenous nerve comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the saphenous nerve comprises applying to the intact skin of a foot of a subject in need thereof, a non-toed high-length ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

The present application also relates to a method of treating an inflammatory condition of the feet, comprising applying to the intact skin of a foot of a subject in need thereof, a sock, and wherein said sock comprises a dry, non-occlusive, fibrous structure, wherein said sock has been finished with a finishing composition that is a homogeneous dispersion comprising an active pharmaceutical ingredient, a surfactant and a solvent, wherein said inflammatory condition comprises either a locally manifesting inflammatory condition or systemic inflammatory condition and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of locally manifesting inflammatory conditions of the feet include bursitis, dermatomyositis, lichen planus, neuritis, panniculitis, phlebitis, tendonitis and tendinosis in or around the foot. Examples of inflammation of tendons of the feet include Achilles tendinitis or Achilles tendinosis. Examples of inflammation of bursae of the feet include Achilles bursitis.

Examples of systemic inflammatory conditions of the feet include autoimmune diseases, gout, manifestations of inflammatory bowel disease, rheumatic fever, sarcoidosis and vasculitis in or around the foot. Examples of autoimmune diseases of the feet include, juvenile idiopathic arthritis (JIA), mast cell activation syndrome (MCAS), psoriasis, reactive arthritis, rheumatoid arthritis, Sjögren's syndrome and systemic lupus erythematosus (SLE).

In one embodiment of the invention, the method of treating locally manifesting inflammatory conditions of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the feet comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating bursitis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating bursitis of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating bursitis of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating bursitis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating bursitis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating bursitis of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating bursitis of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating bursitis of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating bursitis of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides to said subject, either locally or systemically, or both. In a preferred embodiment, the active pharmaceutical ingredient is an NSAID.

In one embodiment of the invention, the method of treating Achilles bursitis comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles bursitis comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles bursitis comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles bursitis comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles bursitis comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles bursitis comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles bursitis comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles bursitis comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles bursitis comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating dermatomyositis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of dermatomyositis of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating lichen planus of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating lichen planus of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating panniculitis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the feet comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating phlebitis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating phlebitis of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating phlebitis of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating phlebitis of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating phlebitis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating phlebitis of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating phlebitis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating phlebitis of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating phlebitis of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating phlebitis of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating phlebitis of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating phlebitis of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendonitis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendonitis of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendonitis of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendonitis of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendonitis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendonitis of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides to said subject, either locally or systemically, or both. In a preferred embodiment, the active pharmaceutical ingredient is an NSAID.

In one embodiment of the invention, the method of treating tendonitis of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendonitis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendonitis of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendonitis of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendonitis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendonitis of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendonitis of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendonitis of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendonitis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendonitis of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendonitis of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendonitis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendonitis of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendonitis of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendinosis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendinosis of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendinosis of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides to said subject, either locally or systemically, or both. In a preferred embodiment, the active pharmaceutical ingredient is an NSAID.

In one embodiment of the invention, the method of treating tendinosis of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendinosis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendinosis of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendinosis of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendinosis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendinosis of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendinosis of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendinosis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendinosis of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendinosis of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is an NSAID.

In one embodiment of the invention, the method of treating Achilles tendinosis of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendinosis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendinosis of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendinosis of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendinosis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendinosis of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating Achilles tendinosis of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about embodiment, the active pharmaceutical ingredient is an NSAID.

In one embodiment of the invention, the method of treating systemic inflammatory conditions of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the feet comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating autoimmune diseases of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the feet comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating juvenile idiopathic arthritis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a DMARD. In another preferred embodiment, the active pharmaceutical ingredient is an NSAID.

In one embodiment of the invention, the method of treating juvenile idiopathic arthritis of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a DMARD. In another preferred embodiment, the active pharmaceutical ingredient is an NSAID.

In one embodiment of the invention, the method of treating juvenile idiopathic arthritis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a DMARD. In another preferred embodiment, the active pharmaceutical ingredient is an NSAID.

In one embodiment of the invention, the method of treating juvenile idiopathic arthritis of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating mast cell activation syndrome of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating mast cell activation syndrome of the feet comprises applying to the intact skin of a foot of a subject in need thereof, a toed sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating mast cell activation syndrome of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating mast cell activation syndrome of the feet comprises applying to the intact skin of a foot of a subject in need thereof, a toed ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating mast cell activation syndrome of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating mast cell activation syndrome of the feet comprises applying to the intact skin of a foot of a subject in need thereof, a toed mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating mast cell activation syndrome of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating mast cell activation syndrome of the feet comprises applying to the intact skin of a foot of a subject in need thereof, a toed knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating mast cell activation syndrome of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating mast cell activation syndrome of the feet comprises applying to the intact skin of a foot of a subject in need thereof, a toed high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating psoriasis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, a non-toed high-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating psoriasis of the feet comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene

In one embodiment of the invention, the method of treating reactive arthritis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating reactive arthritis of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating reactive arthritis of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating reactive arthritis of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating reactive arthritis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating reactive arthritis of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating reactive arthritis of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating reactive arthritis of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating reactive arthritis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating reactive arthritis of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating reactive arthritis of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating reactive arthritis of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating reactive arthritis of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating reactive arthritis of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating reactive arthritis of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating reactive arthritis of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating Sjögren's syndrome of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating Sjögren's syndrome of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating Sjögren's syndrome of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating Sjögren's syndrome of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating gout of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating gout of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating gout of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating gout of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating gout of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating gout of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating gout of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating gout of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating gout of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating gout of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating gout of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating gout of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating gout of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time subject, either locally or systemic ally, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating gout of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating gout of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating gout of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating manifestations of inflammatory bowel disease of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating rheumatic fever of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID.

In one embodiment of the invention, the method of treating rheumatic fever of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical subject, either locally or systemically, or both. In a preferred embodiment, the active pharmaceutical ingredient is a corticosteroid. In another preferred embodiment, the active pharmaceutical ingredient is an NSAID.

In one embodiment of the invention, the method of treating rheumatic fever of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating rheumatic fever of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an NSAID.

In one embodiment of the invention, the method of treating rheumatic fever of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time subject, either locally or systemic ally, or both. In a preferred embodiment, the active pharmaceutical ingredient is a corticosteroid. In another preferred embodiment, the active pharmaceutical ingredient is an NSAID.

In one embodiment of the invention, the method of treating rheumatic fever of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an NSAID.

In one embodiment of the invention, the method of treating rheumatic fever of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an NSAID.

In one embodiment of the invention, the method of treating rheumatic fever of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 embodiment, the active pharmaceutical ingredient is a corticosteroid. In another preferred embodiment, the active pharmaceutical ingredient is an NSAID.

In one embodiment of the invention, the method of treating sarcoidosis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating vasculitis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both In a preferred embodiment, the active pharmaceutical ingredient is an anti-metabolite. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the feet comprises applying to the intact skin of a foot of a subject in need thereof, a non-toed mid-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the feet comprises applying to the intact skin of a foot of a subject in need thereof, a non-toed knee-length sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemic ally, or both. In a preferred embodiment, the active pharmaceutical ingredient is a corticosteroid. In another preferred embodiment, the active pharmaceutical ingredient is an immunosuppressant.

The present application also relates to a method of treating an infective condition of the feet, comprising applying to the intact skin of a foot of a subject in need thereof, a sock, and wherein said sock comprises a dry, non-occlusive, fibrous structure, wherein said sock has been finished with a finishing composition that is a homogeneous dispersion comprising an active pharmaceutical ingredient, a surfactant and a solvent, wherein said infective condition comprises either a mycotic infection, a bacterial infection, a parasitic infection or a viral infection and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of mycotic infections of the feet include tinea pedis or onychomycosis in or around the foot.

Examples of bacterial infections of the feet include folliculitis, impetigo, erysipelas, cellulitis and infections associated with minor cuts, scrapes or burns in or around the foot.

Examples of parasitic infection of the feet include scabies, leishmaniasis and hookworm infections in or around the foot.

Examples of viral infections of the feet include those associated with manifestations of human papillomavirus in or around the foot.

In one embodiment of the invention, the method of treating tinea pedis comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating tinea pedis.

In one embodiment of the invention, the method of treating tinea pedis comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating tinea pedis.

In one embodiment of the invention, the method of treating tinea pedis comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical subject, either locally or systemically, or both. In a preferred embodiment, the active pharmaceutical ingredient is an agent effective in treating tinea pedis.

In one embodiment of the invention, the method of treating tinea pedis comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating tinea pedis.

In one embodiment of the invention, the method of treating tinea pedis comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating tinea pedis.

In one embodiment of the invention, the method of treating tinea pedis comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating tinea pedis.

In one embodiment of the invention, the method of treating onychomycosis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating onychomycosis.

In one embodiment of the invention, the method of treating onychomycosis of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating onychomycosis.

In one embodiment of the invention, the method of treating onychomycosis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating onychomycosis.

In one embodiment of the invention, the method of treating onychomycosis of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating onychomycosis.

In one embodiment of the invention, the method of treating folliculitis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the feet comprises applying to the intact skin of a foot of a subject in need thereof, a mid-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the feet comprises applying to the intact skin of a foot of a subject in need thereof, a knee-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the feet comprises applying to the intact skin of a foot of a subject in need thereof, a high-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 embodiment, the active pharmaceutical ingredient is an agent effective in treating folliculitis.

In one embodiment of the invention, the method of treating impetigo of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the feet comprises applying to the intact skin of a foot of a subject in need thereof, a mid-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the feet comprises applying to the intact skin of a foot of a subject in need thereof, a knee-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the feet comprises applying to the intact skin of a foot of a subject in need thereof, a high-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is an agent effective in treating impetigo.

In one embodiment of the invention, the method of treating erysipelas of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the feet comprises applying to the intact skin of a foot of a subject in need thereof, an ankle toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides to said subject, either locally or systemically, or both. In a preferred embodiment, the active pharmaceutical ingredient is an agent effective in treating erysipelas.

In one embodiment of the invention, the method of treating erysipelas of the feet comprises applying to the intact skin of a foot of a subject in need thereof, a mid-length toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the feet comprises applying to the intact skin of a foot of a subject in need thereof, a knee-length toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the feet comprises applying to the intact skin of a foot of a subject in need thereof, a high-length toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the feet comprises applying to the intact skin of a foot of a subject in need thereof, a mid-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the feet comprises applying to the intact skin of a foot of a subject in need thereof, a knee-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating cellulitis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating cellulitis.

In one embodiment of the invention, the method of treating cellulitis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating cellulitis.

In one embodiment of the invention, the method of treating cellulitis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating cellulitis.

In one embodiment of the invention, the method of treating cellulitis of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating cellulitis.

In one embodiment of the invention, the method of treating cellulitis of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating cellulitis.

In one embodiment of the invention, the method of treating cellulitis of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating cellulitis.

In one embodiment of the invention, the method of treating cellulitis of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating cellulitis.

In one embodiment of the invention, the method of treating cellulitis of the feet comprises applying to the intact skin of a foot of a subject in need thereof, a mid-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating cellulitis.

In one embodiment of the invention, the method of treating cellulitis of the feet comprises applying to the intact skin of a foot of a subject in need thereof, a knee-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating cellulitis.

In one embodiment of the invention, the method of treating cellulitis of the feet comprises applying to the intact skin of a foot of a subject in need thereof, a high-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 embodiment, the active pharmaceutical ingredient is an agent effective in treating cellulitis.

In one embodiment of the invention, the method of treating infections associated with minor cuts, scrapes or burns of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is an agent effective in treating infections associated with minor cuts, scrapes or burns.

In one embodiment of the invention, the method of treating infections associated with minor cuts, scrapes or burns of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is an agent effective in treating infections associated with minor cuts, scrapes or burns.

In one embodiment of the invention, the method of treating infections associated with minor cuts, scrapes or burns of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the feet comprises applying to the intact skin of a foot of a subject in need thereof, a mid-length toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the feet comprises applying to the intact skin of a foot of a subject in need thereof, a knee-length toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the feet comprises applying to the intact skin of a foot of a subject in need thereof, a high-length toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the feet comprises applying to the intact skin of a foot of a subject in need thereof, a mid-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the feet comprises applying to the intact skin of a foot of a subject in need thereof, a knee-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the feet comprises applying to the intact skin of a foot of a subject in need thereof, a high-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically systemically, or both. In a preferred embodiment, the active pharmaceutical ingredient is an agent effective in treating infections associated with minor cuts, scrapes or burns.

In one embodiment of the invention, the method of treating infections scabies of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections scabies.

In one embodiment of the invention, the method of treating infections scabies of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections scabies.

In one embodiment of the invention, the method of treating infections scabies of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections scabies.

In one embodiment of the invention, the method of treating infections scabies of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections scabies.

In one embodiment of the invention, the method of treating infections leishmaniasis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections leishmaniasis.

In one embodiment of the invention, the method of treating infections leishmaniasis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections leishmaniasis.

In one embodiment of the invention, the method of treating infections leishmaniasis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 embodiment, the active pharmaceutical ingredient is an agent effective in treating infections leishmaniasis.

In one embodiment of the invention, the method of treating infections leishmaniasis of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections leishmaniasis.

In one embodiment of the invention, the method of treating infections leishmaniasis of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections leishmaniasis.

In one embodiment of the invention, the method of treating infections leishmaniasis of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is an agent effective in treating infections leishmaniasis.

In one embodiment of the invention, the method of treating infections leishmaniasis of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections leishmaniasis.

In one embodiment of the invention, the method of treating infections leishmaniasis of the feet comprises applying to the intact skin of a foot of a subject in need thereof, a mid-length toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections leishmaniasis.

In one embodiment of the invention, the method of treating infections leishmaniasis of the feet comprises applying to the intact skin of a foot of a subject in need thereof, a knee-length toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is an agent effective in treating infections leishmaniasis.

In one embodiment of the invention, the method of treating infections leishmaniasis of the feet comprises applying to the intact skin of a foot of a subject in need thereof, a high-length toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections leishmaniasis.

In one embodiment of the invention, the method of treating infections leishmaniasis of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections leishmaniasis.

In one embodiment of the invention, the method of treating infections leishmaniasis of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is an agent effective in treating infections leishmaniasis.

In one embodiment of the invention, the method of treating infections leishmaniasis of the feet comprises applying to the intact skin of a foot of a subject in need thereof, a mid-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections leishmaniasis.

In one embodiment of the invention, the method of treating infections leishmaniasis of the feet comprises applying to the intact skin of a foot of a subject in need thereof, a knee-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections leishmaniasis.

In one embodiment of the invention, the method of treating infections leishmaniasis of the feet comprises applying to the intact skin of a foot of a subject in need thereof, a high-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is an agent effective in treating infections leishmaniasis.

In one embodiment of the invention, the method of treating infections hookworm infections of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections hookworm infections.

In one embodiment of the invention, the method of treating infections hookworm infections of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections hookworm infections.

In one embodiment of the invention, the method of treating infections hookworm infections of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections hookworm infections.

In one embodiment of the invention, the method of treating infections hookworm infections of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections hookworm infections.

In one embodiment of the invention, the method of treating manifestations of human papillomavirus of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections human papillomavirus.

In one embodiment of the invention, the method of treating manifestations of human papillomavirus of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections human papillomavirus.

In one embodiment of the invention, the method of treating manifestations of human papillomavirus of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections human papillomavirus.

In one embodiment of the invention, the method of treating manifestations of human papillomavirus of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections human papillomavirus.

In one embodiment of the invention, the method of treating manifestations of human papillomavirus of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections human papillomavirus.

In one embodiment of the invention, the method of treating manifestations of human papillomavirus of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections human papillomavirus.

In one embodiment of the invention, the method of treating manifestations of human papillomavirus of the feet comprises applying to the intact skin of a foot of a subject in need thereof, an ankle toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections human papillomavirus.

In one embodiment of the invention, the method of treating manifestations of human papillomavirus of the feet comprises applying to the intact skin of a foot of a subject in need thereof, a non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections human papillomavirus.

In one embodiment of the invention, the method of treating manifestations of human papillomavirus of the feet comprises applying to the intact skin of a foot of a subject in need thereof, a non-toed ankle sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections human papillomavirus.

In one embodiment of the invention, the method of treating manifestations of human papillomavirus of the feet comprises applying to the intact skin of a foot of a subject in need thereof, a mid-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections human papillomavirus.

In one embodiment of the invention, the method of treating manifestations of human papillomavirus of the feet comprises applying to the intact skin of a foot of a subject in need thereof, a knee-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections human papillomavirus.

In one embodiment of the invention, the method of treating manifestations of human papillomavirus of the feet comprises applying to the intact skin of a foot of a subject in need thereof, a high-length non-toed sock, wherein said non-toed sock comprises a dry, non-occlusive, fibrous structure, and wherein said non-toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections human papillomavirus.

The present application also relates to a method of treating a dermatological condition of the feet, comprising applying to the intact skin of a foot of a subject in need thereof, a sock, and wherein said sock comprises a dry, non-occlusive, fibrous structure, wherein said sock has been finished with a finishing composition that is a homogeneous dispersion comprising an active pharmaceutical ingredient, a surfactant and a solvent, wherein said dermatological condition comprises either an allergic condition, a cutaneous malignancy, a keratosis or a corticosteroid-responsive dermatoses and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of an allergic condition of the feet include atopic dermatitis or urticaria in or around the foot.

Examples of a keratosis of the feet include hyperkeratosis in or around the foot.

Examples of a cutaneous malignancy of the feet include AIDS-related Kaposi's sarcoma in or around the foot.

In one embodiment of the invention, the method of treating an allergic condition of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the feet comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating atopic dermatitis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the feet comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating urticaria of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the feet comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating a cutaneous malignancy of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib

In one embodiment of the invention, the method of treating a cutaneous malignancy of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib

In one embodiment of the invention, the method of treating a cutaneous malignancy of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib

In one embodiment of the invention, the method of treating a cutaneous malignancy of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating a cutaneous malignancy of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib

In one embodiment of the invention, the method of treating a cutaneous malignancy of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating a cutaneous malignancy of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating a cutaneous malignancy of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating a cutaneous malignancy of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating a cutaneous malignancy of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating a cutaneous malignancy of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating a cutaneous malignancy of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating a cutaneous malignancy of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating a cutaneous malignancy of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating a cutaneous malignancy of the feet comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating AIDS-related Kaposi's sarcoma of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is alitretinoin.

In one embodiment of the invention, the method of treating AIDS-related Kaposi's sarcoma of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is alitretinoin.

In one embodiment of the invention, the method of treating AIDS-related Kaposi's sarcoma of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is alitretinoin.

In one embodiment of the invention, the method of treating AIDS-related Kaposi's sarcoma of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is alitretinoin.

In one embodiment of the invention, the method of treating AIDS-related Kaposi's sarcoma of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is alitretinoin.

In one embodiment of the invention, the method of treating AIDS-related Kaposi's sarcoma of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is alitretinoin.

In one embodiment of the invention, the method of treating AIDS-related Kaposi's sarcoma of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is alitretinoin.

In one embodiment of the invention, the method of treating AIDS-related Kaposi's sarcoma of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is alitretinoin.

In one embodiment of the invention, the method of treating AIDS-related Kaposi's sarcoma of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is alitretinoin.

In one embodiment of the invention, the method of treating AIDS-related Kaposi's sarcoma of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is alitretinoin.

In one embodiment of the invention, the method of treating AIDS-related Kaposi's sarcoma of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is alitretinoin.

In one embodiment of the invention, the method of treating AIDS-related Kaposi's sarcoma of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is alitretinoin.

In one embodiment of the invention, the method of treating AIDS-related Kaposi's sarcoma of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is alitretinoin.

In one embodiment of the invention, the method of treating AIDS-related Kaposi's sarcoma of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is alitretinoin.

In one embodiment of the invention, the method of treating AIDS-related Kaposi's sarcoma of the feet comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is alitretinoin.

In one embodiment of the invention, the method of treating a keratosis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the feet comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating hyperkeratosis of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the feet comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating a corticosteroid-responsive dermatoses of the feet comprises applying to the intact skin of a foot of a subject in need thereof, a sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the feet comprises applying to the intact skin of a foot of a subject in need thereof, an ankle sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the feet comprises applying to the intact skin of a foot of a subject in need thereof, a mid-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the feet comprises applying to the intact skin of a foot of a subject in need thereof, a knee-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the feet comprises applying to the intact skin of a foot of a subject in need thereof, a high-length sock, wherein said sock comprises a dry, non-occlusive, fibrous structure, and wherein said sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the feet comprises applying to the intact skin of a foot of a subject in need thereof, a toed sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the feet comprises applying to the intact skin of a foot of a subject in need thereof, a toed ankle sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the feet comprises applying to the intact skin of a foot of a subject in need thereof, a toed mid-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the feet comprises applying to the intact skin of a foot of a subject in need thereof, a toed knee-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a corticosteroid.

In one embodiment of the invention, the method of treating a corticosteroid-responsive dermatoses of the feet comprises applying to the intact skin of a foot of a subject in need thereof, a toed high-length sock, wherein said toed sock comprises a dry, non-occlusive, fibrous structure, and wherein said toed sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the feet comprises applying to the intact skin of a foot of a subject in need thereof, a toeless sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the feet comprises applying to the intact skin of a foot of a subject in need thereof, a toeless ankle sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the feet comprises applying to the intact skin of a foot of a subject in need thereof, a toeless mid-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the feet comprises applying to the intact skin of a foot of a subject in need thereof, a toeless knee-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the feet comprises applying to the intact skin of a foot of a subject in need thereof, a toeless high-length sock, wherein said toeless sock comprises a dry, non-occlusive, fibrous structure, and wherein said toeless sock has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

The present application also relates to a method of treating a painful condition of the knee, comprising applying to the intact skin of a knee of a subject in need thereof, a knee brace, and wherein said knee brace comprises a dry, non-occlusive, fibrous structure, wherein said knee brace has been finished with a finishing composition that is a homogeneous dispersion comprising an active pharmaceutical ingredient, a surfactant and a solvent, wherein said painful condition comprises either a superficial nociceptive pain, a deep nociceptive pain, a supra-spinal central neuropathic pain, a spinal central neuropathic pain, a peripheral neuropathic pain or a mixed central and peripheral neuropathic pain and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of superficial nociceptive pain of the knee include the pain associated with abrasions, cuts, bruises, burns, sunburns or insect bites in or around the knee.

Examples of deep nociceptive pain of the knee include the pain associated with musculoskeletal, post-operative and inflammatory response related pain in or around the knee. Examples of musculoskeletal pain of the knee include the pain associated with sprains, tears to ligaments, tendons or muscle tissue, bone fractures, inflammation of tendons, joints, bursae or fascia and degenerative changes to the musculoskeletal system in or around the knee. Examples of the pain associated with sprains of the knee include the pain associated with quadriceps expansion syndrome and damage to ligaments such as the anterior or posterior cruciate and medial and lateral collateral ligaments. Examples of the pain associated with inflammation of tendons of the knee include the pain associated with patellar tendinitis or patellar tendinosis. Examples of the pain associated with inflammation of bursae of the knee include the pain associated with suprapatellar bursitis, prepatellar bursitis, pes anserine bursitis and infrapatellar bursitis. Examples of the pain associated with inflammation of fascia of the knee include the pain associated with iliotibial band syndrome. Examples of the pain associated with degenerative changes to the musculoskeletal system of the knee include the pain associated with arthritis of the knee.

Examples of central neuropathic pain of the knee include the pain associated with spinal or supra-spinal lesions to the central nervous system which manifest in or around the knee. Examples of the pain of the knee associated with supra-spinal lesions to the central nervous system include the pain associated with central venous thrombosis, with a cerebral tumor, with a traumatic brain lesion, with multiple sclerosis, with Parkinson's disease and the pain occurring after a stroke or a thalamotomy, as presenting on or around the knee. Examples of the pain of the knee associated with spinal lesions to the central nervous system include the pain associated with, with a spinal cord injury, with complications of spinal surgery, with ischemic lesions of the spinal cord, with radiation or HIV associated myelopathy, as presenting on or around the knee.

Examples of peripheral neuropathic pain of the knee include the pain associated with lesions and compressions to the peripheral nervous system in or around the knee. Examples of the pain associated with lesions to peripheral nerves of the knee include the pain associated with length dependent neuropathy, diabetic neuropathy, lupus neuropathy, Sjogren's syndrome associated neuropathy, hypothyroidism associated neuropathy, HIV related neuropathy, alcoholic neuropathy and inflammatory demyelinating polyradiuloneuropathy, in or around the knee. Examples of the pain associated with compressions to peripheral nerves, nerve plexuses and nerve roots of the knee include meralgia paresthetica and entrapment of the femoral or saphenous nerves. Examples of the pain associated with lesions to nerve roots and posterior ganglia which manifest in or around the knee include the pain associated with postherpetic neuralgia and nerve root avulsions, as presenting in or around the knee. Other examples of peripheral neuropathic pain of the knee include the pain associated with paraneoplastic peripheral neuropathy and ganglionopathy, with complications of chemotherapy, radiotherapy and surgery and both Type 1 and Type 2 Complex Regional Pain Syndromes, as presenting in or around the knee.

In one embodiment of the invention, the method of treating the pain associated with abrasions, cuts, bruises, burns, sunburns and insect bites of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating deep nociceptive pain of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating pain of the knee associated with quadriceps expansion syndrome comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating pain of the knee associated with knee sprain comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating pain of the knee associated with damage to the anterior cruciate ligament comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating pain of the knee associated with damage to the posterior cruciate ligament comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating pain of the knee associated with damage to the medial collateral ligament comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating pain of the knee associated with damage to the lateral collateral ligament comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating pain of the knee associated with patellar tendinitis comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating pain of the knee associated with patellar tendinosis comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating pain of the knee associated with suprapatellar bursitis comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating pain of the knee associated with prepatellar bursitis comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating pain of the knee associated with pes anserine bursitis comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating pain of the knee associated with infrapatellar bursitis comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating pain of the knee associated with iliotibial band syndrome comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating pain of the knee associated with arthritis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the knee associated with supra-spinal lesions to the central nervous system comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the knee associated with spinal lesions to the central nervous system comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating peripheral neuropathic pain of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating mixed central and peripheral neuropathic pain of the knee comprises applying to the intact skin of a knee of a subject in need thereof, an ankle knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating the pain associated with length dependent neuropathies of the knee comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the knee comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the knee comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Sjogren's syndrome associated neuropathies of the knee comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the knee comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the knee comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with alcoholic neuropathies of the knee comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the knee comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the knee comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the knee comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with meralgia paresthetica comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment of the femoral nerve comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment of the saphenous nerve comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

The present application also relates to a method of treating an inflammatory condition of the knee, comprising applying to the intact skin of a knee of a subject in need thereof, a knee band, and wherein said knee band comprises a dry, non-occlusive, fibrous structure, wherein said knee band has been finished with a finishing composition that is a homogeneous dispersion comprising an active pharmaceutical ingredient, a surfactant and a solvent, wherein said inflammatory condition comprises either a locally manifesting inflammatory condition or systemic inflammatory condition and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of locally manifesting inflammatory conditions of the knee include bursitis, dermatomyositis, neuritis, panniculitis, phlebitis, tendonitis and tendinosis in or around the knee. Examples of inflammation of tendons of the knee include patellar tendinitis or patellar tendinosis. Examples of inflammation of bursae of the knee include suprapatellar bursitis, prepatellar bursitis, pes anserine bursitis and infrapatellar bursitis.

Examples of systemic inflammatory conditions of the knee include autoimmune diseases, gout, manifestations of inflammatory bowel disease, rheumatic fever, sarcoidosis and vasculitis in or around the knee. Examples of autoimmune diseases of the knee include juvenile idiopathic arthritis (JIA), mast cell activation syndrome (MCAS), psoriasis, reactive arthritis and Sjögren's syndrome.

In one embodiment of the invention, the method of treating locally manifesting inflammatory conditions of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating bursitis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating suprapatellar bursitis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides to said subject, either locally or systemically, or both. In a preferred embodiment, the active pharmaceutical ingredient is an NSAID.

In one embodiment of the invention, the method of treating prepatellar bursitis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating pes anserine bursitis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating infrapatellar bursitis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating dermatomyositis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating panniculitis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating phlebitis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendonitis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating patellar tendonitis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendinosis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating patellar tendinosis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating systemic inflammatory conditions of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating autoimmune diseases of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating juvenile idiopathic arthritis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating mast cell activation syndrome of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating psoriasis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating reactive arthritis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating Sjögren's syndrome of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating gout of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating manifestations of inflammatory bowel disease of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating pseudogout of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID. In another preferred embodiment, the active pharmaceutical ingredient is a colchicine.

In one embodiment of the invention, the method of treating rheumatic fever of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID.

In one embodiment of the invention, the method of treating sarcoidosis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating vasculitis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant.

The present application also relates to a method of treating an infective condition of the knee, comprising applying to the intact skin of a knee of a subject in need thereof, a knee brace, and wherein said knee brace comprises a dry, non-occlusive, fibrous structure, wherein said knee brace has been finished with a finishing composition that is a homogeneous dispersion comprising an active pharmaceutical ingredient, a surfactant and a solvent, wherein said infective condition comprises either a bacterial infection or a parasitic infection and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of bacterial infections of the knee include folliculitis, impetigo, erysipelas, cellulitis and infections associated with minor cuts, scrapes or burns in or around the knee.

Examples of parasitic infection of the knee include leishmaniasis and hookworm infections in or around the knee.

In one embodiment of the invention, the method of treating folliculitis of the knee comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating impetigo of the knee comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating erysipelas of the knee comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating cellulitis of the knee comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical subject, either locally or systemically, or both. In a preferred embodiment, the active pharmaceutical ingredient is an agent effective in treating cellulitis.

In one embodiment of the invention, the method of treating infections associated with minor cuts, scrapes or burns of the knee comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating leishmaniasis of the knee comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating leishmaniasis.

In one embodiment of the invention, the method of treating hookworm infections of the knee comprises applying to the intact skin of a knee of a subject in need thereof, a knee brace, wherein said knee brace comprises a dry, non-occlusive, fibrous structure, and wherein said knee brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating hookworm infections.

The present application also relates to a method of treating a dermatological condition of the knee, comprising applying to the intact skin of a knee of a subject in need thereof, a knee band, and wherein said knee band comprises a dry, non-occlusive, fibrous structure, wherein said knee band has been finished with a finishing composition that is a homogeneous dispersion comprising an active pharmaceutical ingredient, a surfactant and a solvent, wherein said dermatological condition comprises either an allergic condition, a cutaneous malignancy, a keratosis, a cutaneous condition secondary to an external cause or a corticosteroid-responsive dermatoses and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of an allergic condition of the knee include atopic dermatitis or urticaria in or around the knee.

Examples of a keratosis of the knee include hyperkeratosis in or around the knee.

Examples of a cutaneous malignancy of the knee include AIDS-related Kaposi's sarcoma in or around the knee.

Examples of a cutaneous condition secondary to an external cause of the knee include post surgical scaring in or around the knee.

In one embodiment of the invention, the method of treating an allergic condition of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating atopic dermatitis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating urticaria of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating a cutaneous malignancy of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating AIDS-related Kaposi's sarcoma of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is alitretinoin.

In one embodiment of the invention, the method of treating a keratosis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating hyperkeratosis of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating a cutaneous condition secondary to an external cause of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating post surgical scaring of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating a corticosteroid-responsive dermatoses of the knee comprises applying to the intact skin of a knee of a subject in need thereof, a knee band, wherein said knee band comprises a dry, non-occlusive, fibrous structure, and wherein said knee band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

The present application also relates to a method of treating a painful condition of the shoulder, comprising applying to the intact skin of a shoulder of a subject in need thereof, a shoulder brace, and wherein said shoulder brace comprises a dry, non-occlusive, fibrous structure, wherein said shoulder brace has been finished with a finishing composition that is a homogeneous dispersion comprising an active pharmaceutical ingredient, a surfactant and a solvent, wherein said painful condition comprises either a superficial nociceptive pain, a deep nociceptive pain, a supra-spinal central neuropathic pain, a spinal central neuropathic pain, a peripheral neuropathic pain or a mixed central and peripheral neuropathic pain and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of superficial nociceptive pain of the shoulder include the pain associated with abrasions, cuts, bruises, burns, sunburns or insect bites in or around the shoulder.

Examples of deep nociceptive pain of the shoulder include the pain associated with musculoskeletal, post-operative and inflammatory response related pain in or around the shoulder. Examples of musculoskeletal pain of the shoulder include the pain associated with sprains, tears to ligaments, tendons or muscle tissue, bone fractures, inflammation of tendons, joints, bursae or fascia and degenerative changes to the musculoskeletal system in or around the shoulder.

Examples of the pain associated with sprains include the pain associated with acromioclavicular joint dysfunction. Examples of the pain associated with tears or inflammation of tendons of the shoulder include the pain associated with rotator cuff disorders and biceps tendonitis. Examples of the pain associated with inflammation of bursae of the shoulder include the pain associated with subdeltoid bursitis and scapulocostal syndrome. Examples of the pain associated with inflammation of fascia of the shoulder include the pain associated with adhesive capsulitis. Examples of the pain associated with degenerative changes to the musculoskeletal system of the shoulder include the pain associated with arthritis of the shoulder and the acromioclavicular joint.

Examples of central neuropathic pain of the shoulder include the pain associated with spinal or supra-spinal lesions to the central nervous system which manifest in or around the shoulder. Examples of the pain of the shoulder associated with supra-spinal lesions to the central nervous system include the pain associated with central venous thrombosis, with a cerebral tumor, with a traumatic brain lesion, with multiple sclerosis, with Parkinson's disease and the pain occurring after a stroke or a thalamotomy, as presenting on or around the shoulder. Examples of the pain of the shoulder associated with spinal lesions to the central nervous system include the pain associated with, with a spinal cord injury, with complications of spinal surgery, with ischemic lesions of the spinal cord, with radiation or HIV associated myelopathy, as presenting on or around the shoulder.

Examples of peripheral neuropathic pain of the shoulder include the pain associated with lesions and compressions to the peripheral nervous system in or around the shoulder. Examples of the pain associated with lesions to peripheral nerves of the shoulder include the pain associated with length dependent neuropathy, diabetic neuropathy, lupus neuropathy, Sjogren's syndrome associated neuropathy, hypothyroidism associated neuropathy, HIV related neuropathy, alcoholic neuropathy and inflammatory demyelinating polyradiuloneuropathy, in or around the shoulder. Examples of the pain associated with compressions to peripheral nerves, nerve plexuses and nerve roots of the shoulder include entrapment of the suprascapular nerve and the brachial plexus. Examples of the pain associated with lesions to nerve roots and posterior ganglia which manifest in or around the shoulder include the pain associated with postherpetic neuralgia and nerve root avulsions, as presenting in or around the shoulder. Other examples of peripheral neuropathic pain of the shoulder include the pain associated with paraneoplastic peripheral neuropathy and ganglionopathy, with complications of chemotherapy, radiotherapy and surgery and both Type 1 and Type 2 Complex Regional Pain Syndromes, as presenting in or around the shoulder.

In one embodiment of the invention, the method of treating the pain associated with abrasions, cuts, bruises, burns, sunburns and insect bites of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating deep nociceptive pain of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with acromioclavicular joint dysfunction comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with rotator cuff disorders comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with biceps tendonitis comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with subdeltoid bursitis comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with subdeltoid bursitis and scapulocostal syndrome comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with adhesive capsulitis comprises applying to the intact skin of a shoulder of a subject in need thereof, a shoulder band, wherein said shoulder band comprises a dry, non-occlusive, fibrous structure, and wherein said shoulder band has been finished with a finishing composition that is a and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the acromioclavicular joint comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the shoulder associated with supra-spinal lesions to the central nervous system comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the shoulder associated with spinal lesions to the central nervous system comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating peripheral neuropathic pain of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with entrapment of the suprascapular nerve comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment of the brachial plexus comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with postherpetic neuralgia of the shoulder comprises applying to the intact skin of a shoulder of a subject in need thereof, a shoulder brace, wherein said shoulder brace comprises a dry, non-occlusive, fibrous structure, and wherein said shoulder brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating mixed central and peripheral neuropathic pain of the shoulder comprises applying to the intact skin of a shoulder of a subject in need thereof, an ankle shoulder brace, wherein said shoulder brace comprises a dry, non-occlusive, fibrous structure, and wherein said shoulder brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

The present application also relates to a method of treating an inflammatory condition of the shoulder, comprising applying to the intact skin of a shoulder of a subject in need thereof, a shoulder band, and wherein said shoulder band comprises a dry, non-occlusive, fibrous structure, wherein said shoulder band has been finished with a finishing composition that is a homogeneous dispersion comprising an active pharmaceutical ingredient, a surfactant and a solvent, wherein said inflammatory condition comprises either a locally manifesting inflammatory condition or systemic inflammatory condition and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of locally manifesting inflammatory conditions of the shoulder include bursitis, dermatomyositis, panniculitis, tendonitis and tendinosis in or around the shoulder. Examples of inflammation of tendons of the shoulder include rotator cuff disorders and biceps tendonitis. Examples of inflammation of bursae of the shoulder include subdeltoid bursitis and scapulocostal syndrome.

Examples of systemic inflammatory conditions of the shoulder include autoimmune diseases, manifestations of inflammatory bowel disease and vasculitis in or around the shoulder. Examples of autoimmune diseases of the shoulder include ankylosing spondylitis, juvenile idiopathic arthritis (JIA), mast cell activation syndrome (MCAS) and Sjögren's syndrome.

In one embodiment of the invention, the method of treating locally manifesting inflammatory conditions of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating bursitis of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating subdeltoid bursitis of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating scapulocostal syndrome comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating dermatomyositis of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating panniculitis of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating tendonitis of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating rotator cuff disorders comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is an NSAID.

In one embodiment of the invention, the method of treating biceps tendonitis of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendinosis of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating systemic inflammatory conditions of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating autoimmune diseases of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating ankylosing spondylitis of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating mast cell activation syndrome of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating Sjögren's syndrome of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating manifestations of inflammatory bowel disease of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating vasculitis of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant.

The present application also relates to a method of treating an infective condition of the shoulder, comprising applying to the intact skin of a shoulder of a subject in need thereof, a shoulder band, and wherein said shoulder band comprises a dry, non-occlusive, fibrous structure, wherein said shoulder band has been finished with a finishing composition that is a homogeneous dispersion comprising an active pharmaceutical ingredient, a surfactant and a solvent, wherein said infective condition comprises a bacterial infection and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. Examples of bacterial infections of the shoulder include folliculitis, impetigo and infections associated with minor cuts, scrapes or burns in or around the shoulder.

In one embodiment of the invention, the method of treating folliculitis of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating impetigo of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating infections associated with minor cuts, scrapes or burns of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

The present application also relates to a method of treating a dermatological condition of the shoulder, comprising applying to the intact skin of a shoulder of a subject in need thereof, a shoulder band, and wherein said shoulder band comprises a dry, non-occlusive, fibrous structure, wherein said shoulder band has been finished with a finishing composition that is a homogeneous dispersion comprising an active pharmaceutical ingredient, a surfactant and a solvent, wherein said dermatological condition comprises either a cutaneous malignancy, a blister or a corticosteroid-responsive dermatoses and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of a cutaneous malignancy of the shoulder include superficial basal cell carcinoma in or around the shoulder.

Examples of a blister of the shoulder include pemphigoid in or around the shoulder.

In one embodiment of the invention, the method of treating a cutaneous malignancy of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating superficial basal cell carcinoma of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating blisters of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating pemphigoid of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a corticosteroid. In another preferred embodiment, the active ingredient is an immunosuppressant. In another preferred embodiment, the active pharmaceutical ingredient is erythromycin or tetracycline.

In one embodiment of the invention, the method of treating a corticosteroid-responsive dermatoses of the shoulder comprises applying to the intact skin of a shoulder of a subject in need thereof, a shoulder band, wherein said shoulder band comprises a dry, non-occlusive, fibrous structure, and wherein said shoulder band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

The present application also relates to a method of treating a painful condition of the elbow, comprising applying to the intact skin of an elbow of a subject in need thereof, an elbow sleeve, and wherein said elbow sleeve comprises a dry, non-occlusive, fibrous structure, wherein said elbow sleeve has been finished with a finishing composition that is a homogeneous dispersion comprising an active pharmaceutical ingredient, a surfactant and a solvent, wherein said painful condition comprises either a superficial nociceptive pain, a deep nociceptive pain, a supra-spinal central neuropathic pain, a spinal central neuropathic pain, a peripheral neuropathic pain or a mixed central and peripheral neuropathic pain and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of superficial nociceptive pain of the elbow include the pain associated with abrasions, cuts, bruises, burns, sunburns or insect bites in or around the elbow.

Examples of deep nociceptive pain of the elbow include the pain associated with musculoskeletal, post-operative and inflammatory response related pain in or around the elbow. Examples of musculoskeletal pain of the elbow include the pain associated with sprains, tears to ligaments, tendons or muscle tissue, bone fractures, inflammation of tendons, joints, bursae or fascia and degenerative changes to the musculoskeletal system in or around the elbow. Examples of the pain associated with tears or inflammation of tendons of the elbow include the pain associated with lateral epicondylitis and medial epicondylitis. Examples of the pain associated with inflammation of bursae of the elbow include the pain associated with olecranon bursitis and cubital bursitis. Examples of the pain associated with inflammation of fascia of the elbow include the pain associated with adhesive capsulitis. Examples of the pain associated with degenerative changes to the musculoskeletal system of the elbow include the pain associated with arthritis of the elbow.

Examples of central neuropathic pain of the elbow include the pain associated with spinal or supra-spinal lesions to the central nervous system which manifest in or around the elbow. Examples of the pain of the elbow associated with supra-spinal lesions to the central nervous system include the pain associated with central venous thrombosis, with a cerebral tumor, with a traumatic brain lesion, with multiple sclerosis, with Parkinson's disease and the pain occurring after a stroke or a thalamotomy, as presenting on or around the elbow. Examples of the pain of the elbow associated with spinal lesions to the central nervous system include the pain associated with, with a spinal cord injury, with complications of spinal surgery, with ischemic lesions of the spinal cord, with radiation or HIV associated myelopathy, as presenting on or around the elbow.

Examples of peripheral neuropathic pain of the elbow include the pain associated with lesions and compressions to the peripheral nervous system in or around the elbow. Examples of the pain associated with lesions to peripheral nerves of the elbow include the pain associated with length dependent neuropathy, diabetic neuropathy, lupus neuropathy, Sjogren's syndrome associated neuropathy, hypothyroidism associated neuropathy, HIV related neuropathy, alcoholic neuropathy and inflammatory demyelinating polyradiuloneuropathy, in or around the elbow. Examples of the pain associated with compressions to peripheral nerves, nerve plexuses and nerve roots of the elbow include compression and entrapment of the ulnar, median and radial nerves. Examples of the pain associated with lesions to nerve roots and posterior ganglia which manifest in or around the elbow include the pain associated with postherpetic neuralgia and nerve root avulsions, as presenting in or around the elbow. Other examples of peripheral neuropathic pain of the elbow include the pain associated with paraneoplastic peripheral neuropathy and ganglionopathy, with complications of chemotherapy, radiotherapy and surgery and both Type 1 and Type 2 Complex Regional Pain Syndromes, as presenting in or around the elbow.

In one embodiment of the invention, the method of treating the pain associated with abrasions, cuts, bruises, burns, sunburns and insect bites of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating deep nociceptive pain of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with lateral epicondylitis comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with medial condylitis comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with olecranon bursitis comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with cubital bursitis comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with adhesive capsulitis comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the elbow associated with supra-spinal lesions to the central nervous system comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the elbow associated with spinal lesions to the central nervous system comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating peripheral neuropathic pain of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating mixed central and peripheral neuropathic pain of the elbow comprises applying to the intact skin of an elbow of a subject in need thereof, an ankle elbow sleeve, wherein said elbow sleeve comprises a dry, non-occlusive, fibrous structure, and wherein said elbow sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating the pain associated with length dependent neuropathies of the elbow comprises applying to the intact skin of an elbow of a subject in need thereof, an elbow sleeve, wherein said elbow sleeve comprises a dry, non-occlusive, fibrous structure, and wherein said elbow sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the elbow comprises applying to the intact skin of an elbow of a subject in need thereof, an elbow sleeve, wherein said elbow sleeve comprises a dry, non-occlusive, fibrous structure, and wherein said elbow sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the elbow comprises applying to the intact skin of an elbow of a subject in need thereof, an elbow sleeve, wherein said elbow sleeve comprises a dry, non-occlusive, fibrous structure, and wherein said elbow sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with Sjogren's syndrome associated neuropathies of the elbow comprises applying to the intact skin of an elbow of a subject in need thereof, an elbow sleeve, wherein said elbow sleeve comprises a dry, non-occlusive, fibrous structure, and wherein said elbow sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the elbow comprises applying to the intact skin of an elbow of a subject in need thereof, an elbow sleeve, wherein said elbow sleeve comprises a dry, non-occlusive, fibrous structure, and wherein said elbow sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the elbow comprises applying to the intact skin of an elbow of a subject in need thereof, an elbow sleeve, wherein said elbow sleeve comprises a dry, non-occlusive, fibrous structure, and wherein said elbow sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with alcoholic neuropathies of the elbow comprises applying to the intact skin of an elbow of a subject in need thereof, an elbow sleeve, wherein said elbow sleeve comprises a dry, non-occlusive, fibrous structure, and wherein said elbow sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the elbow comprises applying to the intact skin of an elbow of a subject in need thereof, an elbow sleeve, wherein said elbow sleeve comprises a dry, non-occlusive, fibrous structure, and wherein said elbow sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the elbow comprises applying to the intact skin of an elbow of a subject in need thereof, an elbow sleeve, wherein said elbow sleeve comprises a dry, non-occlusive, fibrous structure, and wherein said elbow sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the elbow comprises applying to the intact skin of an elbow of a subject in need thereof, an elbow sleeve, wherein said elbow sleeve comprises a dry, non-occlusive, fibrous structure, and wherein said elbow sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with entrapment syndromes of the ulnar nerve comprises applying to the intact skin of an elbow of a subject in need thereof, an elbow sleeve, wherein said elbow sleeve comprises a dry, non-occlusive, fibrous structure, and wherein said elbow sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the median nerve comprises applying to the intact skin of an elbow of a subject in need thereof, an elbow sleeve, wherein said elbow sleeve comprises a dry, non-occlusive, fibrous structure, and wherein said elbow sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the radial nerve comprises applying to the intact skin of an elbow of a subject in need thereof, an elbow sleeve, wherein said elbow sleeve comprises a dry, non-occlusive, fibrous structure, and wherein said elbow sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

The present application also relates to a method of treating an inflammatory condition of the elbow, comprising applying to the intact skin of an elbowan elbow of a subject in need thereof, an elbow band, and wherein said elbow band comprises a dry, non-occlusive, fibrous structure, wherein said elbow band has been finished with a finishing composition that is a homogeneous dispersion comprising an active pharmaceutical ingredient, a surfactant and a solvent, wherein said inflammatory condition comprises either a locally manifesting inflammatory condition or systemic inflammatory condition and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of locally manifesting inflammatory conditions of the elbow include bursitis, dermatomyositis, panniculitis, tendonitis and tendinosis in or around the elbow. Examples of inflammation of tendons of the elbow include lateral epicondylitis and medial epicondylitis. Examples of inflammation of bursae of the elbow include olecranon bursitis and cubital bursitis.

Examples of systemic inflammatory conditions of the elbow include autoimmune diseases, manifestations of inflammatory bowel disease, sarcoidosis and vasculitis in or around the elbow. Examples of autoimmune diseases of the elbow include mast cell activation syndrome (MCAS), psoriasis, reactive arthritis and Sjögren's syndrome.

In one embodiment of the invention, the method of treating locally manifesting inflammatory conditions of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating bursitis of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating olecranon bursitis of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating cubital bursitis of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating dermatomyositis of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating panniculitis of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating tendonitis of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating lateral epicondylitis comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating medial epicondylitis comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendinosis of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating systemic inflammatory conditions of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating autoimmune diseases of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating mast cell activation syndrome of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating psoriasis of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating reactive arthritis of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating Sjögren's syndrome of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating manifestations of inflammatory bowel disease of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating sarcoidosis of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating vasculitis of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant.

The present application also relates to a method of treating an infective condition of the elbow, comprising applying to the intact skin of an elbow of a subject in need thereof, an elbow band, and wherein said elbow band comprises a dry, non-occlusive, fibrous structure, wherein said elbow band has been finished with a finishing composition that is a homogeneous dispersion comprising an active pharmaceutical ingredient, a surfactant and a solvent, wherein said infective condition comprises either a bacterial infection or a parasitic infection and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of bacterial infections of the elbow include folliculitis, impetigo, erysipelas and infections associated with minor cuts, scrapes or burns in or around the elbow.

Examples of parasitic infection of the elbow include leishmaniasis in or around the elbow.

In one embodiment of the invention, the method of treating folliculitis of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating impetigo of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating erysipelas of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating cellulitis of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating cellulitis.

In one embodiment of the invention, the method of treating infections associated with minor cuts, scrapes or burns of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating leishmaniasis of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating leishmaniasis.

The present application also relates to a method of treating a dermatological condition of the elbow, comprising applying to the intact skin of an elbow of a subject in need thereof, an elbow band, and wherein said elbow band comprises a dry, non-occlusive, fibrous structure, wherein said elbow band has been finished with a finishing composition that is a homogeneous dispersion comprising an active pharmaceutical ingredient, a surfactant and a solvent, wherein said infective condition comprises either an allergic condition, a blister, a cutaneous condition secondary to an external cause or a corticosteroid-responsive dermatoses and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of an allergic condition of the elbow include atopic dermatitis or urticaria in or around the elbow.

Examples of a blister of the elbow include pemphigoid in or around the elbow.

Examples of a cutaneous condition secondary to an external cause of the elbow include post surgical scaring in or around the elbow.

In one embodiment of the invention, the method of treating an allergic condition of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating atopic dermatitis of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating urticaria of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating blisters of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating pemphigoid of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin or tetracycline.

In one embodiment of the invention, the method of treating a cutaneous condition secondary to an external cause of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating post surgical scaring of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating a corticosteroid-responsive dermatoses of the elbow comprises applying to the intact skin of an elbow of a subject in need thereof, an elbow band, wherein said elbow band comprises a dry, non-occlusive, fibrous structure, and wherein said elbow band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

The present application also relates to a method of treating a painful condition of the wrist, comprising applying to the intact skin of a wrist of a subject in need thereof, a wrist band, and wherein said wrist band comprises a dry, non-occlusive, fibrous structure, wherein said wrist band has been finished with a finishing composition that is a homogeneous dispersion comprising an active pharmaceutical ingredient, a surfactant and a solvent, wherein said painful condition comprises either a superficial nociceptive pain, a deep nociceptive pain, a supra-spinal central neuropathic pain, a spinal central neuropathic pain, a peripheral neuropathic pain or a mixed central and peripheral neuropathic pain and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of superficial nociceptive pain of the wrist include the pain associated with abrasions, cuts, bruises, burns, sunburns or insect bites in or around the wrist.

Examples of deep nociceptive pain of the wrist include the pain associated with musculoskeletal, post-operative and inflammatory response related pain in or around the wrist. Examples of musculoskeletal pain of the wrist include the pain associated with sprains, tears to ligaments, tendons or muscle tissue, bone fractures, inflammation of tendons, joints, bursae or fascia and degenerative changes to the musculoskeletal system in or around the wrist. Examples of the pain associated with sprains include the pain associated with repetitive stress of the wrist. Examples of the pain associated with degenerative changes to the musculoskeletal system of the wrist include the pain associated with arthritis of the wrist.

Examples of central neuropathic pain of the wrist include the pain associated with spinal or supra-spinal lesions to the central nervous system which manifest in or around the wrist.

Examples of the pain of the wrist associated with supra-spinal lesions to the central nervous system include the pain associated with central venous thrombosis, with a cerebral tumor, with a traumatic brain lesion, with multiple sclerosis, with Parkinson's disease and the pain occurring after a stroke or a thalamotomy, as presenting on or around the wrist. Examples of the pain of the wrist associated with spinal lesions to the central nervous system include the pain associated with, with a spinal cord injury, with complications of spinal surgery, with ischemic lesions of the spinal cord, with radiation or HIV associated myelopathy, as presenting on or around the wrist.

Examples of peripheral neuropathic pain of the wrist include the pain associated with lesions and compressions to the peripheral nervous system in or around the wrist. Examples of the pain associated with lesions to peripheral nerves of the wrist include the pain associated with length dependent neuropathy, diabetic neuropathy, lupus neuropathy, Sjogren's syndrome associated neuropathy, hypothyroidism associated neuropathy, HIV related neuropathy, alcoholic neuropathy and inflammatory demyelinating polyradiuloneuropathy, in or around the wrist.

Examples of the pain associated with compressions to peripheral nerves, nerve plexuses and nerve roots of the wrist include carpal tunnel syndrome. Examples of the pain associated with lesions to nerve roots and posterior ganglia which manifest in or around the wrist include the pain associated with postherpetic neuralgia and nerve root avulsions, as presenting in or around the wrist. Other examples of peripheral neuropathic pain of the wrist include the pain associated with paraneoplastic peripheral neuropathy and ganglionopathy, with complications of chemotherapy, radiotherapy and surgery and both Type 1 and Type 2 Complex Regional Pain Syndromes, as presenting in or around the wrist.

In one embodiment of the invention, the method of treating the pain associated with abrasions, cuts, bruises, burns, sunburns and insect bites of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating deep nociceptive pain of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with repetitive stress of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the wrist associated with supra-spinal lesions to the central nervous system comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the wrist associated with spinal lesions to the central nervous system comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating peripheral neuropathic pain of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating mixed central and peripheral neuropathic pain of the wrist comprises applying to the intact skin of a wrist of a subject in need thereof, an ankle wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating the pain associated with carpal tunnel syndrome comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

The present application also relates to a method of treating an inflammatory condition of the wrist, comprising applying to the intact skin of a wrist of a subject in need thereof, a wrist band, and wherein said wrist band comprises a dry, non-occlusive, fibrous structure, wherein said wrist band has been finished with a finishing composition that is a homogeneous dispersion comprising an active pharmaceutical ingredient, a surfactant and a solvent, wherein said inflammatory condition comprises either a locally manifesting inflammatory condition or systemic inflammatory condition and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of locally manifesting inflammatory conditions of the wrist include dermatomyositis, lichen planus, panniculitis, tendonitis and tendinosis in or around the wrist.

Examples of systemic inflammatory conditions of the wrist include autoimmune diseases, gout, manifestations of inflammatory bowel disease, pseudogout, sarcoidosis and vasculitis in or around the wrist. Examples of autoimmune diseases of the wrist include juvenile idiopathic arthritis (JIA), mast cell activation syndrome (MCAS), psoriasis, rheumatoid arthritis, Sjögren's syndrome and systemic lupus erythematosus (SLE).

In one embodiment of the invention, the method of treating locally manifesting inflammatory conditions of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating bursitis of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating dermatomyositis of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating lichen planus of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating panniculitis of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating tendonitis of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendinosis of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating systemic inflammatory conditions of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating autoimmune diseases of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating juvenile idiopathic arthritis of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating mast cell activation syndrome of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating psoriasis of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating rheumatoid arthritis of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating Sjögren's syndrome of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating gout of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating manifestations of inflammatory bowel disease of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating pseudogout of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID. In another preferred embodiment, the active pharmaceutical ingredient is a colchicine.

In one embodiment of the invention, the method of treating sarcoidosis of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating vasculitis of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant.

The present application also relates to a method of treating an infective condition of the wrist, comprising applying to the intact skin of a wrist of a subject in need thereof, a wrist band, and wherein said wrist band comprises a dry, non-occlusive, fibrous structure, wherein said wrist band has been finished with a finishing composition that is a homogeneous dispersion comprising an active pharmaceutical ingredient, a surfactant and a solvent, wherein said infective condition comprises either a bacterial infection or a parasitic infection and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of bacterial infections of the wrist include folliculitis, impetigo, erysipelas and infections associated with minor cuts, scrapes or burns in or around the wrist.

Examples of parasitic infection of the wrist include scabies in or around the wrist.

In one embodiment of the invention, the method of treating folliculitis of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating impetigo of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating erysipelas of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating infections associated with minor cuts, scrapes or burns of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating scabies of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating scabies.

The present application also relates to a method of treating a dermatological condition of the wrist, comprising applying to the intact skin of a wrist of a subject in need thereof, a wrist band, and wherein said wrist band comprises a dry, non-occlusive, fibrous structure, wherein said wrist band has been finished with a finishing composition that is a homogeneous dispersion comprising an active pharmaceutical ingredient, a surfactant and a solvent, wherein said dermatological condition comprises either an allergic condition, a keratosis or a corticosteroid-responsive dermatoses and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of an allergic condition of the wrist include atopic dermatitis or urticaria in or around the wrist.

Examples of a keratosis of the wrist include actinic keratosis in or around the wrist.

In one embodiment of the invention, the method of treating an allergic condition of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating atopic dermatitis of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating a keratosis of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating actinic keratosis of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating a corticosteroid-responsive dermatoses of the wrist comprises applying to the intact skin of a wrist of a subject in need thereof, a wrist band, wherein said wrist band comprises a dry, non-occlusive, fibrous structure, and wherein said wrist band has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

The present application also relates to a method of treating a painful condition of the hand, comprising applying to the intact skin of a hand of a subject in need thereof, a glove, and wherein said glove comprises a dry, non-occlusive, fibrous structure, wherein said glove has been finished with a finishing composition that is a homogeneous dispersion comprising an active pharmaceutical ingredient, a surfactant and a solvent, wherein said painful condition comprises either a superficial nociceptive pain, a deep nociceptive pain, a supra-spinal central neuropathic pain, a spinal central neuropathic pain, a peripheral neuropathic pain or a mixed central and peripheral neuropathic pain and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of superficial nociceptive pain of the hand include the pain associated with abrasions, cuts, bruises, burns, sunburns or insect bites in or around the hand.

Examples of deep nociceptive pain of the hand include the pain associated with musculoskeletal, post-operative and inflammatory response related pain in or around the hand. Examples of musculoskeletal pain of the hand include the pain associated with sprains, tears to ligaments, tendons or muscle tissue, bone fractures, inflammation of tendons, joints, bursae or fascia and degenerative changes to the musculoskeletal system in or around the hand. Examples of the pain associated with sprains include the pain associated with repetitive stress of the hand. Examples of the pain associated with tears or inflammation of tendons of the hand include the pain associated with De Quervain's syndrome, digital flexor tendinitis and tenosynovitis. Examples of the pain associated with inflammation of fascia of the hand include the pain associated with Dupuytren's contracture. Examples of the pain associated with degenerative changes to the musculoskeletal system of the hand include the pain associated with arthritis of the hand.

Examples of central neuropathic pain of the hand include the pain associated with spinal or supra-spinal lesions to the central nervous system which manifest in or around the hand. Examples of the pain of the hand associated with supra-spinal lesions to the central nervous system include the pain associated with central venous thrombosis, with a cerebral tumor, with a traumatic brain lesion, with multiple sclerosis, with Parkinson's disease and the pain occurring after a stroke or a thalamotomy, as presenting on or around the hand. Examples of the pain of the hand associated with spinal lesions to the central nervous system include the pain associated with, with a spinal cord injury, with complications of spinal surgery, with ischemic lesions of the spinal cord, with radiation or HIV associated myelopathy, as presenting on or around the hand.

Examples of peripheral neuropathic pain of the hand include the pain associated with lesions and compressions to the peripheral nervous system in or around the hand. Examples of the pain associated with lesions to peripheral nerves of the hand include the pain associated with length dependent neuropathy, diabetic neuropathy, lupus neuropathy, Sjogren's syndrome associated neuropathy, hypothyroidism associated neuropathy, HIV related neuropathy, alcoholic neuropathy and inflammatory demyelinating polyradiuloneuropathy, in or around the hand. Examples of the pain associated with compressions to peripheral nerves, nerve plexuses and nerve roots of the hand include entrapment of the ulnar, median and radial nerves. Examples of the pain associated with lesions to nerve roots and posterior ganglia which manifest in or around the hand include the pain associated with postherpetic neuralgia and nerve root avulsions, as presenting in or around the hand. Other examples of peripheral neuropathic pain of the hand include the pain associated with paraneoplastic peripheral neuropathy and ganglionopathy, with complications of chemotherapy, radiotherapy and surgery and both Type 1 and Type 2 Complex Regional Pain Syndromes, as presenting in or around the hand.

In one embodiment of the invention, the method of treating the pain associated with abrasions, cuts, bruises, burns, sunburns and insect bites of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the hand comprises applying to the intact skin of a hand of a subject in need thereof, a finger glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the hand comprises applying to the intact skin of a hand of a subject in need thereof, a fingerless glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating deep nociceptive pain of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the hand comprises applying to the intact skin of a hand of a subject in need thereof, a finger glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the hand comprises applying to the intact skin of a hand of a subject in need thereof, a fingerless glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with repetitive stress of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with repetitive stress of the hand comprises applying to the intact skin of a hand of a subject in need thereof, a finger glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with repetitive stress of the hand comprises applying to the intact skin of a hand of a subject in need thereof, a fingerless glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with repetitive stress of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with repetitive stress of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with repetitive stress of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with repetitive stress of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with repetitive stress of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with De Quervain's syndrome comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with De Quervain's syndrome comprises applying to the intact skin of a hand of a subject in need thereof, a finger glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with De Quervain's syndrome comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with De Quervain's syndrome comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with digital flexor tendinitis comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with digital flexor tendinitis comprises applying to the intact skin of a hand of a subject in need thereof, a finger glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with digital flexor tendinitis comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with digital flexor tendinitis comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with digital flexor tenosynovitis comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with digital flexor tenosynovitis comprises applying to the intact skin of a hand of a subject in need thereof, a finger glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with digital flexor tenosynovitis comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with digital flexor tenosynovitis comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Dupuytren's contracture comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Dupuytren's contracture comprises applying to the intact skin of a hand of a subject in need thereof, a finger glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Dupuytren's contracture comprises applying to the intact skin of a hand of a subject in need thereof, a fingerless glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Dupuytren's contracture comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Dupuytren's contracture comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Dupuytren's contracture comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Dupuytren's contracture comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Dupuytren's contracture comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Dupuytren's contracture comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Dupuytren's contracture comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Dupuytren's contracture comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with Dupuytren's contracture comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the hand comprises applying to the intact skin of a hand of a subject in need thereof, a finger glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the hand comprises applying to the intact skin of a hand of a subject in need thereof, a fingerless glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the hand associated with supra-spinal lesions to the central nervous system comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with supra-spinal lesions to the central nervous system comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with supra-spinal lesions to the central nervous system comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with supra-spinal lesions to the central nervous system comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with supra-spinal lesions to the central nervous system comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with supra-spinal lesions to the central nervous system comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with supra-spinal lesions to the central nervous system comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with supra-spinal lesions to the central nervous system comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with supra-spinal lesions to the central nervous system comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with supra-spinal lesions to the central nervous system comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with supra-spinal lesions to the central nervous system comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with supra-spinal lesions to the central nervous system comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with spinal lesions to the central nervous system comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with spinal lesions to the central nervous system comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with spinal lesions to the central nervous system comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with spinal lesions to the central nervous system comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with spinal lesions to the central nervous system comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with spinal lesions to the central nervous system comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with spinal lesions to the central nervous system comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with spinal lesions to the central nervous system comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with spinal lesions to the central nervous system comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with spinal lesions to the central nervous system comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the hand associated with spinal lesions to the central nervous system comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating peripheral neuropathic pain of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating mixed central and peripheral neuropathic pain of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating the pain associated with length dependent neuropathies of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with length dependent neuropathies of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with diabetic neuropathies of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with lupus neuropathies of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of the pain associated with Sjogren's syndrome associated neuropathies of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of the pain associated with Sjogren's syndrome associated neuropathies of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of the pain associated with Sjogren's syndrome associated neuropathies of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of the pain associated with Sjogren's syndrome associated neuropathies of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of the pain associated with Sjogren's syndrome associated neuropathies of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of the pain associated with Sjogren's syndrome associated neuropathies of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of the pain associated with Sjogren's syndrome associated neuropathies of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of the pain associated with Sjogren's syndrome associated neuropathies of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of the pain associated with Sjogren's syndrome associated neuropathies of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of the pain associated with Sjogren's syndrome associated neuropathies of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of the pain associated with Sjogren's syndrome associated neuropathies of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of the pain associated with Sjogren's syndrome associated neuropathies of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with hypothyroidism associated neuropathies of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with HIV related neuropathies of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with alcoholic neuropathies of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with alcoholic neuropathies of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with alcoholic neuropathies of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with alcoholic neuropathies of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with alcoholic neuropathies of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with alcoholic neuropathies of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with alcoholic neuropathies of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with alcoholic neuropathies of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with alcoholic neuropathies of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with alcoholic neuropathies of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with alcoholic neuropathies of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with alcoholic neuropathies of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with inflammatory demyelinating polyradiuloneuropathies of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the hand comprises applying to the intact skin of a hand of a subject in need thereof, an elbow glove, wherein said glove comprises a dry, non-occlusive, fibrous structure, and wherein said glove has been finished with a finishing composition that is a and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with paraneoplastic peripheral neuropathies of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with chemotherapy as manifesting in or around the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with chemotherapy as manifesting in or around the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with chemotherapy as manifesting in or around the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with chemotherapy as manifesting in or around the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with chemotherapy as manifesting in or around the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with chemotherapy as manifesting in or around the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with chemotherapy as manifesting in or around the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with chemotherapy as manifesting in or around the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method the pain associated with chemotherapy as manifesting in or around the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with entrapment syndromes of the ulnar nerve comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the ulnar nerve comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the ulnar nerve comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the ulnar nerve comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the ulnar nerve comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the ulnar nerve comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the ulnar nerve comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the ulnar nerve comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the ulnar nerve comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the ulnar nerve comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the ulnar nerve comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the ulnar nerve comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the median nerve comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the median nerve comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the median nerve comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the median nerve comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the median nerve comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the median nerve comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the median nerve comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the median nerve comprises applying to the intact skin of a hand of a subject in need thereof, a short mitten, wherein said mitten comprises a dry, non-occlusive, fibrous structure, and wherein said mitten has been finished with a finishing composition that is a and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the median nerve comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the median nerve comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the median nerve comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the median nerve comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the radial nerve comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the radial nerve comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the radial nerve comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the radial nerve comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the radial nerve comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the radial nerve comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the radial nerve comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the radial nerve comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the radial nerve comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the radial nerve comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the radial nerve comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with entrapment syndromes of the radial nerve comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

The present application also relates to a method of treating an inflammatory condition of the hand, comprising applying to the intact skin of a hand of a subject in need thereof, a glove, and wherein said glove comprises a dry, non-occlusive, fibrous structure, wherein said glove has been finished with a finishing composition that is a homogeneous dispersion comprising an active pharmaceutical ingredient, a surfactant and a solvent, wherein said inflammatory condition comprises either a locally manifesting inflammatory condition or systemic inflammatory condition and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of locally manifesting inflammatory conditions of the hand include dermatomyositis, panniculitis, tendonitis and tendinosis in or around the hand. Examples of inflammation of tendons of the hand include De Quervain's syndrome, digital flexor tendinitis and tenosynovitis.

Examples of systemic inflammatory conditions of the hand include autoimmune diseases, gout, manifestations of inflammatory bowel disease, pseudogout, sarcoidosis and vasculitis in or around the hand. Examples of autoimmune diseases of the hand include juvenile idiopathic arthritis (HA), mast cell activation syndrome (MCAS), psoriasis, rheumatoid arthritis, Sjögren's syndrome and systemic lupus erythematosus (SLE).

In one embodiment of the invention, the method of treating locally manifesting inflammatory conditions of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating dermatomyositis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating panniculitis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide

In one embodiment of the invention, the method of treating panniculitis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide

In one embodiment of the invention, the method of treating panniculitis of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide

In one embodiment of the invention, the method of treating panniculitis of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide

In one embodiment of the invention, the method of treating panniculitis of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide

In one embodiment of the invention, the method of treating panniculitis of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide

In one embodiment of the invention, the method of treating panniculitis of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide

In one embodiment of the invention, the method of treating panniculitis of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide

In one embodiment of the invention, the method of treating panniculitis of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide

In one embodiment of the invention, the method of treating panniculitis of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide

In one embodiment of the invention, the method of treating panniculitis of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide

In one embodiment of the invention, the method of treating panniculitis of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or bothIn a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide

In one embodiment of the invention, the method of treating tendonitis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendonitis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendonitis of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendonitis of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendonitis of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendonitis of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating De Quervain's syndrome comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating De Quervain's syndrome comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating De Quervain's syndrome comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating De Quervain's syndrome comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating De Quervain's syndrome comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides to said subject, either locally or systemically, or both. In a preferred embodiment, the active pharmaceutical ingredient is an NSAID.

In one embodiment of the invention, the method of treating De Quervain's syndrome comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating digital flexor tendinitis comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating digital flexor tendinitis comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating digital flexor tendinitis comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating digital flexor tendinitis comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating digital flexor tendinitis comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating digital flexor tendinitis comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tenosynovitis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tenosynovitis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tenosynovitis of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tenosynovitis of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tenosynovitis of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tenosynovitis of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendinosis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendinosis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendinosis of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendinosis of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 embodiment, the active pharmaceutical ingredient is an NSAID.

In one embodiment of the invention, the method of treating tendinosis of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendinosis of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating systemic inflammatory conditions of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about pharmaceutical ingredient to said subject, either locally or systemically, or bothIn a preferred embodiment, the active pharmaceutical ingredient is a corticosteroid. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating autoimmune diseases of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or bothIn a preferred embodiment, the active pharmaceutical ingredient is a corticosteroid. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating juvenile idiopathic arthritis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a DMARD. In another preferred embodiment, the active pharmaceutical ingredient is an NSAID.

In one embodiment of the invention, the method of treating juvenile idiopathic arthritis of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or bothIn a preferred embodiment, the active pharmaceutical ingredient is a corticosteroid. In a preferred embodiment, the active pharmaceutical ingredient is a DMARD. In another preferred embodiment, the active pharmaceutical ingredient is an NSAID.

In one embodiment of the invention, the method of treating mast cell activation syndrome of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating mast cell activation syndrome of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating mast cell activation syndrome of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating mast cell activation syndrome of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating mast cell activation syndrome of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating mast cell activation syndrome of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating psoriasis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating psoriasis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating psoriasis of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating psoriasis of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating psoriasis of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating psoriasis of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating psoriasis of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating psoriasis of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating psoriasis of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating psoriasis of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating psoriasis of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating psoriasis of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or bothIn a preferred embodiment, the active pharmaceutical ingredient is a corticosteroid. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating rheumatoid arthritis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating rheumatoid arthritis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating rheumatoid arthritis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating rheumatoid arthritis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating rheumatoid arthritis of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating rheumatoid arthritis of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating Sjögren's syndrome of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating Sjögren's syndrome of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating Sjögren's syndrome of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating Sjögren's syndrome of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating Sjögren's syndrome of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating Sjögren's syndrome of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or bothIn a preferred embodiment, the active pharmaceutical ingredient is a corticosteroid. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating Gout of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating Gout of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating Gout of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating Gout of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating Gout of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating Gout of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating Gout of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating Gout of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time subject, either locally or systemically, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating Gout of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating Gout of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating Gout of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating Gout of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or bothIn a preferred embodiment, the active pharmaceutical ingredient is a corticosteroid. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating manifestations of inflammatory bowel disease of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating manifestations of inflammatory bowel disease of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating pseudogout of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID. In another preferred embodiment, the active pharmaceutical ingredient is a colchicine.

In one embodiment of the invention, the method of treating pseudogout of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID. In another preferred embodiment, the active pharmaceutical ingredient is a colchicine.

In one embodiment of the invention, the method of treating pseudogout of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID. In another preferred embodiment, the active pharmaceutical ingredient is a colchicine.

In one embodiment of the invention, the method of treating pseudogout of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID. In another preferred embodiment, the active pharmaceutical ingredient is a colchicine.

In one embodiment of the invention, the method of treating pseudogout of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID. In another preferred embodiment, the active pharmaceutical ingredient is a colchicine.

In one embodiment of the invention, the method of treating pseudogout of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID. In another preferred embodiment, the active pharmaceutical ingredient is a colchicine.

In one embodiment of the invention, the method of treating pseudogout of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID. In another preferred embodiment, the active pharmaceutical ingredient is a colchicine.

In one embodiment of the invention, the method of treating pseudogout of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID. In another preferred embodiment, the active pharmaceutical ingredient is a colchicine.

In one embodiment of the invention, the method of treating pseudogout of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID. In another preferred embodiment, the active pharmaceutical ingredient is a colchicine.

In one embodiment of the invention, the method of treating pseudogout of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID. In another preferred embodiment, the active pharmaceutical ingredient is a colchicine.

In one embodiment of the invention, the method of treating pseudogout of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID. In another preferred embodiment, the active pharmaceutical ingredient is a colchicine.

In one embodiment of the invention, the method of treating pseudogout of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID. In another preferred embodiment, the active pharmaceutical ingredient is a colchicine.

In one embodiment of the invention, the method of treating sarcoidosis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating sarcoidosis of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating vasculitis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time subject, either locally or systemically, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant.

In one embodiment of the invention, the method of treating vasculitis of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical subject, either locally or systemically, or both. In a preferred embodiment, the active pharmaceutical ingredient is a corticosteroid. In another preferred embodiment, the active pharmaceutical ingredient is an immunosuppressant.

The present application also relates to a method of treating an infective condition of the hand, comprising applying to the intact skin of a hand of a subject in need thereof, a glove, and wherein said glove comprises a dry, non-occlusive, fibrous structure, wherein said glove has been finished with a finishing composition that is a homogeneous dispersion comprising an active pharmaceutical ingredient, a surfactant and a solvent, wherein said infective condition comprises either a mycotic infection, a bacterial infection, a parasitic infection or a viral infection and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of mycotic infections of the hand include onychomycosis in or around the hand.

Examples of bacterial infections of the hand include folliculitis, impetigo, erysipelas and infections associated with minor cuts, scrapes or burns in or around the hand.

Examples of parasitic infection of the hand include scabies in or around the hand.

Examples of viral infections of the hand include those associated with manifestations of human papillomavirus in or around the hand.

In one embodiment of the invention, the method of treating onychomycosis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating onychomycosis.

In one embodiment of the invention, the method of treating onychomycosis of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating onychomycosis.

In one embodiment of the invention, the method of treating onychomycosis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating onychomycosis.

In one embodiment of the invention, the method of treating onychomycosis of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating onychomycosis.

In one embodiment of the invention, the method of treating folliculitis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical subject, either locally or systemically, or both. In a preferred embodiment, the active pharmaceutical ingredient is an agent effective in treating folliculitis.

In one embodiment of the invention, the method of treating folliculitis of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the hand comprises applying to the intact skin of a hand of a subject in need thereof, an fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating impetigo of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides to said subject, either locally or systemically, or both. In a preferred embodiment, the active pharmaceutical ingredient is an agent effective in treating impetigo.

In one embodiment of the invention, the method of treating impetigo of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the hand comprises applying to the intact skin of a hand of a subject in need thereof, an fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating impetigo of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating erysipelas of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the hand comprises applying to the intact skin of a hand of a subject in need thereof, an fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating erysipelas of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical subject, either locally or systemically, or both. In a preferred embodiment, the active pharmaceutical ingredient is an agent effective in treating erysipelas.

In one embodiment of the invention, the method of treating erysipelas of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating infections associated with minor cuts, scrapes or burns of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is an agent effective in treating infections associated with minor cuts, scrapes or burns.

In one embodiment of the invention, the method of treating infections associated with minor cuts, scrapes or burns of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the hand comprises applying to the intact skin of a hand of a subject in need thereof, a finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the hand comprises applying to the intact skin of a hand of a subject in need thereof, an fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is an agent effective in treating infections associated with minor cuts, scrapes or burns.

In one embodiment of the invention, the method of treating infections associated with minor cuts, scrapes or burns of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating scabies of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating scabies.

In one embodiment of the invention, the method of treating scabies of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating scabies.

In one embodiment of the invention, the method of treating scabies of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating scabies.

In one embodiment of the invention, the method of treating scabies of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating scabies.

In one embodiment of the invention, the method of treating manifestations of human papillomavirus of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating human papillomavirus.

In one embodiment of the invention, the method of treating manifestations of human papillomavirus of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating human papillomavirus.

In one embodiment of the invention, the method of treating manifestations of human papillomavirus of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating human papillomavirus.

In one embodiment of the invention, the method of treating manifestations of human papillomavirus of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating human papillomavirus.

The present application also relates to a method of treating a dermatological condition of the hand, comprising applying to the intact skin of a hand of a subject in need thereof, a glove, and wherein said glove comprises a dry, non-occlusive, fibrous structure, wherein said glove has been finished with a finishing composition that is a homogeneous dispersion comprising an active pharmaceutical ingredient, a surfactant and a solvent, wherein said dermatological condition comprises either an allergic condition, a blister, a keratosis or a corticosteroid-responsive dermatoses and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of an allergic condition of the hand include atopic dermatitis, contact dermatitis or urticaria in or around the hand.

Examples of a blister of the hand include pemphigus in or around the hand.

Examples of a keratosis of the hand include actinic keratosis or hyperkeratosis in or around the hand.

In one embodiment of the invention, the method of treating an allergic condition of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the hand comprises applying to the intact skin of a hand of a subject in need thereof, an finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating atopic dermatitis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the hand comprises applying to the intact skin of a hand of a subject in need thereof, an finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating atopic dermatitis of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, crisaborole, doxepin, desonide, dexpanthenol, ruxolitinib or suplatast.

In one embodiment of the invention, the method of treating contact dermatitis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is doxepin.

In one embodiment of the invention, the method of treating contact dermatitis of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is doxepin.

In one embodiment of the invention, the method of treating contact dermatitis of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is doxepin.

In one embodiment of the invention, the method of treating contact dermatitis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is doxepin.

In one embodiment of the invention, the method of treating contact dermatitis of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is doxepin.

In one embodiment of the invention, the method of treating contact dermatitis of the hand comprises applying to the intact skin of a hand of a subject in need thereof, an finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is doxepin.

In one embodiment of the invention, the method of treating contact dermatitis of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is doxepin.

In one embodiment of the invention, the method of treating contact dermatitis of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is doxepin.

In one embodiment of the invention, the method of treating contact dermatitis of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is doxepin.

In one embodiment of the invention, the method of treating contact dermatitis of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is doxepin.

In one embodiment of the invention, the method of treating contact dermatitis of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is doxepin.

In one embodiment of the invention, the method of treating contact dermatitis of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is an immunomodulator. In another preferred embodiment, the active pharmaceutical ingredient is doxepin.

In one embodiment of the invention, the method of treating urticaria of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the hand comprises applying to the intact skin of a hand of a subject in need thereof, an finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating blisters of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating blisters of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating blisters of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating blisters of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating blisters of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating blisters of the hand comprises applying to the intact skin of a hand of a subject in need thereof, an finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating blisters of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating blisters of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating blisters of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating blisters of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating blisters of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating blisters of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating pemphigus of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant.

In one embodiment of the invention, the method of treating pemphigus of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant.

In one embodiment of the invention, the method of treating pemphigus of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant.

In one embodiment of the invention, the method of treating pemphigus of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant.

In one embodiment of the invention, the method of treating pemphigus of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant.

In one embodiment of the invention, the method of treating pemphigus of the hand comprises applying to the intact skin of a hand of a subject in need thereof, an finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a corticosteroid. In another preferred embodiment, the active ingredient is an immunosuppressant.

In one embodiment of the invention, the method of treating pemphigus of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant.

In one embodiment of the invention, the method of treating pemphigus of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant.

In one embodiment of the invention, the method of treating pemphigus of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a corticosteroid. In another preferred embodiment, the active ingredient is an immunosuppressant.

In one embodiment of the invention, the method of treating pemphigus of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant.

In one embodiment of the invention, the method of treating pemphigus of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant.

In one embodiment of the invention, the method of treating pemphigus of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant.

In one embodiment of the invention, the method of treating a keratosis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the hand comprises applying to the intact skin of a hand of a subject in need thereof, an finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating actinic keratosis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating actinic keratosis of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating actinic keratosis of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating actinic keratosis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating actinic keratosis of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating actinic keratosis of the hand comprises applying to the intact skin of a hand of a subject in need thereof, an finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating actinic keratosis of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating actinic keratosis of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating actinic keratosis of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating actinic keratosis of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating actinic keratosis of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating actinic keratosis of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating hyperkeratosis of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the hand comprises applying to the intact skin of a hand of a subject in need thereof, an finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating hyperkeratosis of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, calcipotriol, isotretinoin, lactic acid, maxacalcitol, salicylic acid or tretinoin.

In one embodiment of the invention, the method of treating a corticosteroid-responsive dermatoses of the hand comprises applying to the intact skin of a hand of a subject in need thereof, a glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the hand comprises applying to the intact skin of a hand of a subject in need thereof, a short glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the hand comprises applying to the intact skin of a hand of a subject in need thereof, an elbow glove, wherein said glove comprises a dry, non-occlusive, fibrous structure, and wherein said glove has been finished with a finishing composition that is a homogeneous dispersion comprising an active pharmaceutical ingredient, a surfactant and a solvent, and wherein embodiment, the active pharmaceutical ingredient is a corticosteroid.

In one embodiment of the invention, the method of treating a corticosteroid-responsive dermatoses of the hand comprises applying to the intact skin of a hand of a subject in need thereof, a finger glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the hand comprises applying to the intact skin of a hand of a subject in need thereof, a finger short glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the hand comprises applying to the intact skin of a hand of a subject in need thereof, an finger elbow glove, wherein said finger glove comprises a dry, non-occlusive, fibrous structure, and wherein said finger glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the hand comprises applying to the intact skin of a hand of a subject in need thereof, a fingerless glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the hand comprises applying to the intact skin of a hand of a subject in need thereof, a fingerless short glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the hand comprises applying to the intact skin of a hand of a subject in need thereof, a fingerless elbow glove, wherein said fingerless glove comprises a dry, non-occlusive, fibrous structure, and wherein said fingerless glove has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the hand comprises applying to the intact skin of a hand of a subject in need thereof, a mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the hand comprises applying to the intact skin of a hand of a subject in need thereof, a short mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the hand comprises applying to the intact skin of a hand of a subject in need thereof, an elbow mitten, wherein said mitten comprises a dry, non-occlusive, fibrous structure, and wherein said mitten has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

The present application also relates to a method of treating a painful condition of the neck, comprising applying to the intact skin of the neck of a subject in need thereof, a neck sleeve, and wherein said neck sleeve comprises a dry, non-occlusive, fibrous structure, wherein said neck sleeve has been finished with a finishing composition that is a homogeneous dispersion comprising an active pharmaceutical ingredient, a surfactant and a solvent, wherein said painful condition comprises either a superficial nociceptive pain, a deep nociceptive pain, a supra-spinal central neuropathic pain, a spinal central neuropathic pain, a peripheral neuropathic pain or a mixed central and peripheral neuropathic pain and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of superficial nociceptive pain of the neck include the pain associated with abrasions, cuts, bruises, burns, sunburns or insect bites in or around the neck.

Examples of deep nociceptive pain of the neck include the pain associated with musculoskeletal, post-operative and inflammatory response related pain in or around the neck. Examples of musculoskeletal pain of the neck include the pain associated with sprains, tears to ligaments, tendons or muscle tissue, bone fractures, inflammation of tendons, joints, bursae or fascia and degenerative changes to the musculoskeletal system in or around the neck. Examples of the pain associated with sprains include the pain associated with repetitive stress of the neck. Examples of the pain associated with degenerative changes to the musculoskeletal system of the neck include the pain associated with degenerative cervical spine disease.

Examples of central neuropathic pain of the neck include the pain associated with spinal or supra-spinal lesions and compressions to the central nervous system, which manifest in or around the neck. Examples of the pain of the neck associated with supra-spinal lesions to the central nervous system include the pain associated with central venous thrombosis, with a cerebral tumor, with a traumatic brain lesion, with multiple sclerosis, with Parkinson's disease and the pain occurring after a stroke or a thalamotomy, as presenting on or around the neck. Examples of the pain of the neck associated with spinal compressions to the central nervous system include the pain associated with cervical myelopathy. Examples of the pain of the neck associated with spinal lesions to the central nervous system include the pain associated with, with a spinal cord injury, with complications of spinal surgery, with ischemic lesions of the spinal cord, with radiation or HIV associated myelopathy, as presenting on or around the neck.

Examples of peripheral neuropathic pain of the neck include the pain associated with lesions and compressions to the peripheral nervous system in or around the neck. Examples of the pain associated with compressions to peripheral nerves, nerve plexuses and nerve roots of the neck include the pain associated with cervical radiculopathy.

In one embodiment of the invention, the method of treating the pain associated with abrasions, cuts, bruises, burns, sunburns and insect bites of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the neck comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating deep nociceptive pain of the neck comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the neck comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with repetitive stress of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with repetitive stress of the neck comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with repetitive stress of the neck comprises applying to the intact skin of the neck of a subject in need thereof, a neck sleeve, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the neck associated with supra-spinal lesions to the central nervous system comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the neck associated with supra-spinal lesions to the central nervous system comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the neck associated with supra-spinal lesions to the central nervous system comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the neck associated with spinal compressions to the central nervous system comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the neck associated with spinal compressions to the central nervous system comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the neck associated with spinal compressions to the central nervous system comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the neck associated with cervical myelopathy comprises applying to the intact skin of the neck of a subject in need thereof, a neck sleeve, wherein said neck sleeve comprises a dry, non-occlusive, fibrous structure, and wherein said neck sleeve has been finished with a finishing composition that is a and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the neck associated with cervical myelopathy comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the neck associated with cervical myelopathy comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the neck associated with spinal lesions to the central nervous system comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the neck associated with spinal lesions to the central nervous system comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the neck associated with spinal lesions to the central nervous system comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating peripheral neuropathic pain of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the neck comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating mixed central and peripheral neuropathic pain of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the neck comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating the pain associated with compressions to peripheral nerves, nerve plexuses and nerve roots of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with compressions to peripheral nerves, nerve plexuses and nerve roots of the neck comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with compressions to peripheral nerves, nerve plexuses and nerve roots of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with cervical radiculopathy comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with cervical radiculopathy comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with cervical radiculopathy comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

The present application also relates to a method of treating an inflammatory condition of the neck, comprising applying to the intact skin of the neck of a subject in need thereof, a neck sleeve, and wherein said neck sleeve comprises a dry, non-occlusive, fibrous structure, wherein said neck sleeve has been finished with a finishing composition that is a homogeneous dispersion comprising an active pharmaceutical ingredient, a surfactant and a solvent, wherein said inflammatory condition comprises either a locally manifesting inflammatory condition or systemic inflammatory condition and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of locally manifesting inflammatory conditions of the neck include dermatomyositis, and panniculitis in or around the neck.

Examples of systemic inflammatory conditions of the neck include autoimmune diseases and, manifestations of inflammatory bowel disease in or around the neck. Examples of autoimmune diseases of the neck include ankylosing spondylitis and mast cell activation syndrome (MCAS).

In one embodiment of the invention, the method of treating locally manifesting inflammatory conditions of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the neck comprises applying to the intact skin of the neck of a subject in need thereof, a closed neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating dermatomyositis of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the neck comprises applying to the intact skin of the neck of a subject in need thereof, a closed neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating panniculitis of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the neck comprises applying to the intact skin of a closed neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating systemic inflammatory conditions of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the neck comprises applying to the intact skin of the neck of a subject in need thereof, a closed neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating autoimmune diseases of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the neck comprises applying to the intact skin of the neck of a subject in need thereof, a closed neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating ankylosing spondylitis of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a DMARD. In another preferred embodiment, the active pharmaceutical ingredient is an NSAID.

In one embodiment of the invention, the method of treating ankylosing spondylitis of the neck comprises applying to the intact skin of the neck of a subject in need thereof, a closed neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating ankylosing spondylitis of the neckcomprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating mast cell activation syndrome of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating mast cell activation syndrome of the neck comprises applying to the intact skin of the neck of a subject in need thereof, a closed neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating mast cell activation syndrome of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

The present application also relates to a method of treating an infective condition of the neck, comprising applying to the intact skin of the neck of a subject in need thereof, a neck sleeve, and wherein said neck sleeve comprises a dry, non-occlusive, fibrous structure, wherein said neck sleeve has been finished with a finishing composition that is a homogeneous dispersion comprising an active pharmaceutical ingredient, a surfactant and a solvent, wherein said infective condition comprises either a bacterial infection or a parasitic and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of bacterial infections of the neck include folliculitis and infections associated with minor cuts, scrapes or burns in or around the neck.

Examples of parasitic infection of the neck include leishmaniasis in or around the neck.

In one embodiment of the invention, the method of treating folliculitis of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the neck comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating folliculitis of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating infections associated with minor cuts, scrapes or burns of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the neck comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating infections associated with minor cuts, scrapes or burns of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein embodiment, the active pharmaceutical ingredient is an agent effective in treating infections associated with minor cuts, scrapes or burns.

In one embodiment of the invention, the method of treating leishmaniasis of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating leishmaniasis.

In one embodiment of the invention, the method of treating leishmaniasis of the neck comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating leishmaniasis.

In one embodiment of the invention, the method of treating leishmaniasis of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating leishmaniasis.

The present application also relates to a method of treating a dermatological condition of the neck, comprising applying to the intact skin of the neck of a subject in need thereof, a neck sleeve, and wherein said neck sleeve comprises a dry, non-occlusive, fibrous structure, wherein said neck sleeve has been finished with a finishing composition that is a homogeneous dispersion comprising an active pharmaceutical ingredient, a surfactant and a solvent, wherein said dermatological condition comprises either a cutaneous malignancy, a keratosis, a cutaneous condition secondary to an external cause or a corticosteroid-responsive dermatoses and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of a cutaneous malignancy of the neck include superficial basal cell carcinomas in or around the neck.

Examples of a keratosis of the neck include actinic keratosis in or around the neck.

Examples of a cutaneous condition secondary to an external cause of the neck include post surgical scaring in or around the neck.

In one embodiment of the invention, the method of treating a cutaneous malignancy of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating a cutaneous malignancy of the neck comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating a cutaneous malignancy of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating a superficial basal cell carcinoma of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is aminolevulinic acid, fluorouracil, imiquimod, sonidegib, tazarotene or vismodegib.

In one embodiment of the invention, the method of treating a superficial basal cell carcinoma of the neck comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is aminolevulinic acid, fluorouracil, imiquimod, sonidegib, tazarotene or vismodegib.

In one embodiment of the invention, the method of treating a superficial basal cell carcinoma of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is aminolevulinic acid, fluorouracil, imiquimod, sonidegib, tazarotene or vismodegib.

In one embodiment of the invention, the method of treating a keratosis of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the neck comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating actinic keratosis of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating actinic keratosis of the neck comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating actinic keratosis of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating a cutaneous condition secondary to an external cause of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating a cutaneous condition secondary to an external cause of the neck comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating a cutaneous condition secondary to an external cause of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating post surgical scaring of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating post surgical scaring of the neck comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating post surgical scaring of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating a corticosteroid-responsive dermatoses of the neck comprises applying to the intact skin of the neck of a subject in need thereof, a neck sleeve, wherein said neck sleeve comprises a dry, non-occlusive, fibrous structure, and wherein said neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the neck comprises applying to the intact skin of the neck of a subject in need thereof, a closed neck sleeve, wherein said closed neck sleeve comprises a dry, non-occlusive, fibrous structure, and wherein said closed neck sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of the neck comprises applying to the intact skin of the neck of a subject in need thereof, a scarf, wherein said scarf comprises a dry, non-occlusive, fibrous structure, and wherein said scarf has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

The present application also relates to a method of treating a painful condition of the back, comprising applying to the intact skin of the back of a subject in need thereof, a back brace, and wherein said back brace comprises a dry, non-occlusive, fibrous structure, wherein said back brace has been finished with a finishing composition that is a homogeneous dispersion comprising an active pharmaceutical ingredient, a surfactant and a solvent, wherein said painful condition comprises either a superficial nociceptive pain, a deep nociceptive pain, a supra-spinal central neuropathic pain, a spinal central neuropathic pain, a peripheral neuropathic pain or a mixed central and peripheral neuropathic pain and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of superficial nociceptive pain of the back include the pain associated with abrasions, cuts, bruises, burns, sunburns or insect bites in or around the back.

Examples of deep nociceptive pain of the back include the pain associated with musculoskeletal, post-operative and inflammatory response related pain in or around the back. Examples of musculoskeletal pain of the back include the pain associated with sprains, tears to ligaments, tendons or muscle tissue, bone fractures, inflammation of tendons, joints, bursae or fascia and degenerative changes to the musculoskeletal system in or around the back. Examples of the pain associated with sprains include the pain associated with lumbar muscular strain or sprain and scoliosis. Examples of the pain associated with inflammation of the back include the pain associated with a rheumatological condition, including one or more of the following: ankylosing spondylitis, reactive arthritis, psoriatic arthritis, vertebral osteochondritis, polymyalgia rheumatica and fibromyalgia. Examples of the pain associated with degenerative changes to the musculoskeletal system of the back include the pain associated with arthritis of the lumbar spine and osteoporosis.

Examples of central neuropathic pain of the back include the pain associated with spinal or supra-spinal lesions and compressions to the central nervous system, which manifest in or around the back. Examples of the pain of the back associated with supra-spinal lesions to the central nervous system include the pain associated with central venous thrombosis, with a cerebral tumor, with a traumatic brain lesion, with multiple sclerosis, with Parkinson's disease and the pain occurring after a stroke or a thalamotomy, as presenting on or around the back. Examples of the pain of the back associated with spinal compressions to the central nervous system include the pain associated with lumbar myelopathy. Examples of the pain of the back associated with spinal lesions to the central nervous system include the pain associated with, with a spinal cord injury, with complications of spinal surgery, with ischemic lesions of the spinal cord, with radiation or HIV associated myelopathy, as presenting on or around the back.

Examples of peripheral neuropathic pain of the back include the pain associated with lesions and compressions to the peripheral nervous system in or around the back. Examples of the pain associated with lesions to peripheral nerves of the back include the pain associated with length dependent neuropathy, diabetic neuropathy, lupus neuropathy, Sjogren's syndrome associated neuropathy, hypothyroidism associated neuropathy, HIV related neuropathy, alcoholic neuropathy and inflammatory demyelinating polyradiuloneuropathy, in or around the back. Examples of the pain associated with compressions to peripheral nerves, nerve plexuses and nerve roots of the back include the pain associated with lumbar radiculopathy. Examples of the pain associated with lesions to nerve roots and posterior ganglia which manifest in or around the back include the pain associated with postherpetic neuralgia and nerve root avulsions, as presenting in or around the back. Other examples of peripheral neuropathic pain of the back include the pain associated with paraneoplastic peripheral neuropathy and ganglionopathy, with complications of chemotherapy, radiotherapy and surgery and both Type 1 and Type 2 Complex Regional Pain Syndromes, as presenting in or around the back.

In one embodiment of the invention, the method of treating the pain associated with abrasions, cuts, bruises, burns, sunburns and insect bites of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating deep nociceptive pain of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with lumbar muscular strain comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with lumbar muscular sprain comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with scoliosis comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the back associated with ankylosing spondylitis comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the back associated with reactive arthritis comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the back associated with psoriatic arthritis comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the back associated with vertebral osteochondritis comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the back associated with polymyalgia rheumatica comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the back associated with fibromyalgia comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the lumbar spine comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the back associated with osteoporosis comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the back associated with supra-spinal lesions to the central nervous system comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the back associated with spinal compressions to the central nervous system comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the back associated with lumbar myelopathy comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the back associated with spinal lesions to the central nervous system comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating peripheral neuropathic pain of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating mixed central and peripheral neuropathic pain of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating the pain associated with postherpetic neuralgia of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with compressions to peripheral nerves, nerve plexuses and nerve roots of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with lumbar radiculopathy comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

The present application also relates to a method of treating an inflammatory condition of the back, comprising applying to the intact skin of the back of a subject in need thereof, a back brace, and wherein said back brace comprises a dry, non-occlusive, fibrous structure, wherein said back brace has been finished with a finishing composition that is a homogeneous dispersion comprising an active pharmaceutical ingredient, a surfactant and a solvent, wherein said inflammatory condition comprises either a locally manifesting inflammatory condition or systemic inflammatory condition and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of locally manifesting inflammatory conditions of the back include dermatomyositis and panniculitis in or around the back.

Examples of systemic inflammatory conditions of the back include autoimmune diseases and manifestations of inflammatory bowel disease in or around the back. Examples of autoimmune diseases of the back include ankylosing spondylitis, juvenile idiopathic arthritis (JIA), mast cell activation syndrome (MCAS) and psoriasis.

In one embodiment of the invention, the method of treating locally manifesting inflammatory conditions of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating dermatomyositis of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating panniculitis of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating systemic inflammatory conditions of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating autoimmune diseases of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating ankylosing spondylitis of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating mast cell activation syndrome of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating psoriasis of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating manifestations of inflammatory bowel disease of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

The present application also relates to a method of treating an infective condition of the back, comprising applying to the intact skin of the back of a subject in need thereof, a back brace, and wherein said back brace comprises a dry, non-occlusive, fibrous structure, wherein said back brace has been finished with a finishing composition that is a homogeneous dispersion comprising an active pharmaceutical ingredient, a surfactant and a solvent, wherein said infective condition comprises either a mycotic infection, a bacterial infection, a parasitic infection or a viral infection and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of mycotic infections of the back include tinea versicolor and tinea corporis in or around the back.

Examples of bacterial infections of the back include folliculitis, cellulitis and infections associated with minor cuts, scrapes or burns in or around the back.

Examples of parasitic infection of the back include leishmaniasis in or around the back.

Examples of viral infections of the back include those associated with manifestations of varicella zoster virus in or around the back.

In one embodiment of the invention, the method of treating tinea versicolor of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating tinea versicolor.

In one embodiment of the invention, the method of treating tinea corporis of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides to said subject, either locally or systemically, or both. In a preferred embodiment, the active pharmaceutical ingredient is an agent effective in treating tinea corporis.

In one embodiment of the invention, the method of treating folliculitis of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating cellulitis of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating cellulitis.

In one embodiment of the invention, the method of treating infections associated with minor cuts, scrapes or burns of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating leishmaniasis of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating leishmaniasis.

In one embodiment of the invention, the method of treating manifestations of varicella zoster virus of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating varicella zoster virus.

The present application also relates to a method of treating a dermatological condition of the back, comprising applying to the intact skin of the back of a subject in need thereof, a back brace, and wherein said back brace comprises a dry, non-occlusive, fibrous structure, wherein said back brace has been finished with a finishing composition that is a homogeneous dispersion comprising an active pharmaceutical ingredient, a surfactant and a solvent, wherein said dermatological condition comprises either an allergic condition, a cutaneous malignancy, a blister, pruritis, a cutaneous condition secondary to an external cause or a corticosteroid-responsive dermatoses and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of an allergic condition of the back include urticaria in or around the back.

Examples of a cutaneous malignancy of the back include cutaneous t-cell lymphomas in or around the back.

Examples of a blister of the back include pemphigus or pemphigoid in or around the back.

Examples of a cutaneous condition secondary to an external cause of the back include post surgical scaring in or around the back.

In one embodiment of the invention, the method of treating an allergic condition of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating urticaria of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating pruritis of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is a local anesthetic. In another preferred embodiment, the active pharmaceutical ingredient is a topical analgesic drug. In another preferred embodiment, the active pharmaceutical ingredient is cholestyramine, estradiol, isothipendyl, nalfurafine, pseudoephedrine, serlopitant or tradipitant.

In one embodiment of the invention, the method of treating a cutaneous malignancy of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating cutaneous t-cell lymphoma of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, bexarotene, carmustine, denileukin diftitox, etoposide, imiquimod, mechlorethamine, methotrexate, romidepsin, tazarotene or vorinostat.

In one embodiment of the invention, the method of treating blisters of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating pemphigoid of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin or tetracycline.

In one embodiment of the invention, the method of treating pemphigus of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant.

In one embodiment of the invention, the method of treating a cutaneous condition secondary to an external cause of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating post surgical scaring of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating a corticosteroid-responsive dermatoses of the back comprises applying to the intact skin of the back of a subject in need thereof, a back brace, wherein said back brace comprises a dry, non-occlusive, fibrous structure, and wherein said back brace has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

The present application also relates to a method of treating a painful condition of a body appendage, comprising applying to the intact skin of the body appendage of a subject in need thereof, a sleeve, and wherein said sleeve comprises a dry, non-occlusive, fibrous structure, wherein said sleeve has been finished with a finishing composition that is a homogeneous dispersion comprising an active pharmaceutical ingredient, a surfactant and a solvent, wherein said painful condition comprises either a superficial nociceptive pain, a deep nociceptive pain, a supra-spinal central neuropathic pain, a spinal central neuropathic pain, a peripheral neuropathic pain or a mixed central and peripheral neuropathic pain and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of superficial nociceptive pain of a body appendage include the pain associated with abrasions, cuts, bruises, burns, sunburns or insect bites in or around the body appendage.

Examples of deep nociceptive pain of a body appendage include the pain associated with musculoskeletal, post-operative and inflammatory response related pain in or around the body appendage. Examples of musculoskeletal pain of a body appendage include the pain associated with sprains, tears to ligaments, tendons or muscle tissue, bone fractures, inflammation of tendons, joints, bursae or fascia and degenerative changes to the musculoskeletal system in or around the body appendage.

Examples of central neuropathic pain of a body appendage include the pain associated with spinal or supra-spinal lesions and compressions to the central nervous system, which manifest in or around the body appendage. Examples of the pain of a body appendage associated with supra-spinal lesions to the central nervous system include the pain associated with central venous thrombosis, with a cerebral tumor, with a traumatic brain lesion, with multiple sclerosis, with Parkinson's disease and the pain occurring after a stroke or a thalamotomy, as presenting on or around the body appendage. Examples of the pain of a body appendage associated with spinal lesions to the central nervous system include the pain associated with, with a spinal cord injury, with complications of spinal surgery, with ischemic lesions of the spinal cord, with radiation or HIV associated myelopathy, as presenting on or around the body appendage.

Examples of peripheral neuropathic pain of a body appendage include the pain associated with lesions and compressions to the peripheral nervous system in or around the body appendage. Examples of the pain associated with lesions to peripheral nerves of a body appendage include the pain associated with length dependent neuropathy, diabetic neuropathy, lupus neuropathy, Sjogren's syndrome associated neuropathy, hypothyroidism associated neuropathy, HIV related neuropathy, alcoholic neuropathy and inflammatory demyelinating polyradiuloneuropathy, in or around the body appendage. Examples of the pain associated with lesions to nerve roots and posterior ganglia which manifest in or around the body appendage include the pain associated with postherpetic neuralgia and nerve root avulsions, as presenting in or around the body appendage. Other examples of peripheral neuropathic pain of a body appendage include the pain associated with paraneoplastic peripheral neuropathy and ganglionopathy, with complications of chemotherapy, radiotherapy and surgery and both Type 1 and Type 2 Complex Regional Pain Syndromes, as presenting in or around the body appendage.

In one embodiment of the invention, the method of treating the pain associated with abrasions, cuts, bruises, burns, sunburns and insect bites of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating deep nociceptive pain of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of a body appendage associated with supra-spinal lesions to the central nervous system comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of a body appendage associated with spinal lesions to the central nervous system comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating peripheral neuropathic pain of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating mixed central and peripheral neuropathic pain of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

The present application also relates to a method of treating an inflammatory condition of a body appendage, comprising applying to the intact skin of the body appendage of a subject in need thereof, a sleeve, and wherein said sleeve comprises a dry, non-occlusive, fibrous structure, wherein said sleeve has been finished with a finishing composition that is a homogeneous dispersion comprising an active pharmaceutical ingredient, a surfactant and a solvent, wherein said inflammatory condition comprises either a locally manifesting inflammatory condition or systemic inflammatory condition and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of locally manifesting inflammatory conditions of a body appendage include bursitis, dermatomyositis, lichen planus, neuritis, panniculitis, phlebitis, tendonitis and tendinosis in or around a body appendage.

Examples of systemic inflammatory conditions of a body appendage include autoimmune diseases, gout, manifestations of inflammatory bowel disease, pseudogout, rheumatic fever, sarcoidosis and vasculitis in or around a body appendage. Examples of autoimmune diseases of a body appendage include ankylosing spondylitis, juvenile idiopathic arthritis (JIA), mast cell activation syndrome (MCAS), psoriasis, reactive arthritis, rheumatoid arthritis, Sjögren's syndrome and systemic lupus erythematosus (SLE).

In one embodiment of the invention, the method of treating locally manifesting inflammatory conditions of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating systemic inflammatory conditions of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating autoimmune diseases of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

The present application also relates to a method of treating an infective condition of a body appendage, comprising applying to the intact skin of the body appendage of a subject in need thereof, a sleeve, and wherein said sleeve comprises a dry, non-occlusive, fibrous structure, wherein said sleeve has been finished with a finishing composition that is a homogeneous dispersion comprising an active pharmaceutical ingredient, a surfactant and a solvent, wherein said infective condition comprises either a mycotic infection, a bacterial infection, a parasitic infection or a viral infection and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of mycotic infections of a body appendage include tinea versicolor and tinea corporis in or around a body appendage.

Examples of bacterial infections of a body appendage include folliculitis, cellulitis, erysipelas, impetigo and infections associated with minor cuts, scrapes or burns in or around a body appendage.

Examples of parasitic infection of a body appendage include leishmaniasis in or around a body appendage.

Examples of viral infections of a body appendage include those associated with manifestations of varicella zoster virus or human papillomavirus in or around a body appendage.

In one embodiment of the invention, the method of treating tinea versicolor of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating tinea versicolor.

In one embodiment of the invention, the method of treating tinea corporis of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 embodiment, the active pharmaceutical ingredient is an agent effective in treating tinea corporis.

In one embodiment of the invention, the method of treating folliculitis of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating impetigo of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating erysipelas of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating cellulitis of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating cellulitis.

In one embodiment of the invention, the method of treating infections associated with minor cuts, scrapes or burns of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating leishmaniasis of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating leishmaniasis.

In one embodiment of the invention, the method of treating manifestations of varicella zoster virus of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating varicella zoster virus.

In one embodiment of the invention, the method of treating manifestations of human papillomavirus of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating human papillomavirus.

The present application also relates to a method of treating a dermatological condition of a body appendage, comprising applying to the intact skin of the body appendage of a subject in need thereof, a sleeve, and wherein said sleeve comprises a dry, non-occlusive, fibrous structure, wherein said sleeve has been finished with a finishing composition that is a homogeneous dispersion comprising an active pharmaceutical ingredient, a surfactant and a solvent, wherein said dermatological condition comprises either an allergic condition, a cutaneous malignancy, a blister, pruritis, an acneiform eruption, a keratosis, a cutaneous condition secondary to an external cause or a corticosteroid-responsive dermatoses and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of an allergic condition of a body appendage include atopic dermatitis, contact dermatitis or urticaria in or around the body appendage.

Examples of a cutaneous malignancy of a body appendage include cutaneous t-cell lymphomas, superficial basal cell carcinomas or AIDS-related Kaposi's sarcoma in or around the body appendage.

Examples of a blisters of a body appendage include dermatitis herpetiformis, pemphigoid or pemphigus in or around the body appendage.

Examples of a keratosis of a body appendage include actinic keratosis or hyperkeratosis in or around the body appendage.

Examples of a cutaneous condition secondary to an external cause of a body appendage include post surgical scaring in or around the body appendage.

In one embodiment of the invention, the method of treating an allergic condition of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating pruritis of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is a local anesthetic. In another preferred embodiment, the active pharmaceutical ingredient is a topical analgesic drug. In another preferred embodiment, the active pharmaceutical ingredient is cholestyramine, estradiol, isothipendyl, nalfurafine, pseudoephedrine, serlopitant or tradipitant.

In one embodiment of the invention, the method of treating a cutaneous malignancy of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating blisters of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating a keratosis of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a cutaneous condition secondary to an external cause of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating a corticosteroid-responsive dermatoses of a body appendage comprises applying to the intact skin of the body appendage of a subject in need thereof, a sleeve, wherein said sleeve comprises a dry, non-occlusive, fibrous structure, and wherein said sleeve has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

The present application also relates to a method of treating a painful condition of the torso, comprising applying to the intact skin of the torso of a subject in need thereof, a t-shirt, and wherein said t-shirt comprises a dry, non-occlusive, fibrous structure, wherein said t-shirt has been finished with a finishing composition that is a homogeneous dispersion comprising an active pharmaceutical ingredient, a surfactant and a solvent, wherein said painful condition comprises either a superficial nociceptive pain, a deep nociceptive pain, a supra-spinal central neuropathic pain, a spinal central neuropathic pain, a peripheral neuropathic pain or a mixed central and peripheral neuropathic pain and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of superficial nociceptive pain of the torso include the pain associated with abrasions, cuts, bruises, burns, sunburns or insect bites in or around the torso.

Examples of deep nociceptive pain of the torso include the pain associated with musculoskeletal, post-operative and inflammatory response related pain in or around the torso. Examples of musculoskeletal pain of the torso include the pain associated with sprains, tears to ligaments, tendons or muscle tissue, bone fractures, inflammation of tendons, joints, bursae or fascia and degenerative changes to the musculoskeletal system in or around the torso. Examples of the pain associated with sprains include the pain associated with thoracic muscular strain or sprain and scoliosis. Examples of the pain associated with inflammation of the torso include the pain associated with a rheumatological condition, including one or more of the following: ankylosing spondylitis, reactive arthritis, psoriatic arthritis, vertebral osteochondritis, polymyalgia rheumatica and fibromyalgia. Examples of the pain associated with degenerative changes to the musculoskeletal system of the torso include the pain associated with arthritis of the thoracic spine and osteoporosis.

Examples of central neuropathic pain of the torso include the pain associated with spinal or supra-spinal lesions and compressions to the central nervous system, which manifest in or around the torso. Examples of the pain of the torso associated with supra-spinal lesions to the central nervous system include the pain associated with central venous thrombosis, with a cerebral tumor, with a traumatic brain lesion, with multiple sclerosis, with Parkinson's disease and the pain occurring after a stroke or a thalamotomy, as presenting on or around the torso. Examples of the pain of the torso associated with spinal compressions to the central nervous system include the pain associated with thoracic myelopathy. Examples of the pain of the torso associated with spinal lesions to the central nervous system include the pain associated with, with a spinal cord injury, with complications of spinal surgery, with ischemic lesions of the spinal cord, with radiation or HIV associated myelopathy, as presenting on or around the torso.

Examples of peripheral neuropathic pain of the torso include the pain associated with lesions and compressions to the peripheral nervous system in or around the torso. Examples of the pain associated with lesions to peripheral nerves of the torso include the pain associated with length dependent neuropathy, diabetic neuropathy, lupus neuropathy, Sjogren's syndrome associated neuropathy, hypothyroidism associated neuropathy, HIV related neuropathy, alcoholic neuropathy and inflammatory demyelinating polyradiuloneuropathy, in or around the torso. Examples of the pain associated with compressions to peripheral nerves, nerve plexuses and nerve roots of the torso include the pain associated with thoracic radiculopathy. Examples of the pain associated with lesions to nerve roots and posterior ganglia which manifest in or around the torso include the pain associated with postherpetic neuralgia and nerve root avulsions, as presenting in or around the torso. Other examples of peripheral neuropathic pain of the torso include the pain associated with paraneoplastic peripheral neuropathy and ganglionopathy, with complications of chemotherapy, radiotherapy and surgery and both Type 1 and Type 2 Complex Regional Pain Syndromes, as presenting in or around the torso.

In one embodiment of the invention, the method of treating the pain associated with abrasions, cuts, bruises, burns, sunburns and insect bites of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the shoulders and chest comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating the pain associated with abrasions, cuts, bruises, burns, sunburns and insect bites of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating deep nociceptive pain of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the shoulders and chest comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating deep nociceptive pain of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with thoracic muscular strain comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with thoracic muscular strain comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with thoracic muscular sprain comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with thoracic muscular sprain comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with scoliosis comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with scoliosis comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with ankylosing spondylitis comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with ankylosing spondylitis comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with reactive arthritis comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with reactive arthritis comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with psoriatic arthritis comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with psoriatic arthritis comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with vertebral osteochondritis comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with vertebral osteochondritis comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with polymyalgia rheumatica comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with polymyalgia rheumatica comprises applying to the intact skin of the torso of a subject in need thereof, a long-shirt, wherein said long-shirt comprises a dry, non-occlusive, fibrous structure, and wherein said long-shirt has been finished with a finishing composition that is a and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with fibromyalgia comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with fibromyalgia comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the thoracic spine comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the thoracic spine comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with arthritis of the thoracic spine comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with osteoporosis comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with osteoporosis comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with osteoporosis comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with supra-spinal lesions to the central nervous system comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the torso associated with supra-spinal lesions to the central nervous system comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the torso associated with supra-spinal lesions to the central nervous system comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the torso associated with spinal compressions to the central nervous system comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with spinal compressions to the central nervous system comprises applying to the intact skin of the torso of a subject in need thereof, a short-shirt, wherein said t-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with spinal compressions to the central nervous system comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with thoracic myelopathy comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with thoracic myelopathy comprises applying to the intact skin of the torso of a subject in need thereof, a short-shirt, wherein said t-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with thoracic myelopathy comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain of the torso associated with spinal lesions to the central nervous system comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the torso associated with spinal lesions to the central nervous system comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain of the torso associated with spinal lesions to the central nervous system comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating peripheral neuropathic pain of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating peripheral neuropathic pain of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating mixed central and peripheral neuropathic pain of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating mixed central and peripheral neuropathic pain of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating the pain associated with postherpetic neuralgia of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with postherpetic neuralgia of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating the pain associated with compressions to peripheral nerves, nerve plexuses and nerve roots of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with compressions to peripheral nerves, nerve plexuses and nerve roots of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with compressions to peripheral nerves, nerve plexuses and nerve roots of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with thoracic radiculopathy comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with thoracic radiculopathy comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with thoracic radiculopathy comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

The present application also relates to a method of treating an inflammatory condition of the torso, comprising applying to the intact skin of the torso of a subject in need thereof, a t-shirt, and wherein said t-shirt comprises a dry, non-occlusive, fibrous structure, wherein said t-shirt has been finished with a finishing composition that is a homogeneous dispersion comprising an active pharmaceutical ingredient, a surfactant and a solvent, wherein said inflammatory condition comprises either a locally manifesting inflammatory condition or systemic inflammatory condition and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of locally manifesting inflammatory conditions of the torso include dermatomyositis, lichen planus and panniculitis in or around the torso.

Examples of systemic inflammatory conditions of the torso include autoimmune diseases and manifestations of inflammatory bowel disease in or around the torso. Examples of autoimmune diseases of the torso include ankylosing spondylitis, mast cell activation syndrome (MCAS) and psoriasis.

In one embodiment of the invention, the method of treating locally manifesting inflammatory conditions of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating locally manifesting inflammatory conditions of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating dermatomyositis of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating dermatomyositis of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating panniculitis of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating panniculitis of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating systemic inflammatory conditions of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating systemic inflammatory conditions of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating autoimmune diseases of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating autoimmune diseases of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating ankylosing spondylitis of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating ankylosing spondylitis of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a DMARD. In another preferred embodiment, the active pharmaceutical ingredient is an NSAID.

In one embodiment of the invention, the method of treating ankylosing spondylitis of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating mast cell activation syndrome of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating mast cell activation syndrome of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating mast cell activation syndrome of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating psoriasis of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating psoriasis of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating psoriasis of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

The present application also relates to a method of treating an infective condition of the torso, comprising applying to the intact skin of the torso of a subject in need thereof, a t-shirt, and wherein said t-shirt comprises a dry, non-occlusive, fibrous structure, wherein said t-shirt has been finished with a finishing composition that is a homogeneous dispersion comprising an active pharmaceutical ingredient, a surfactant and a solvent, wherein said infective condition comprises either a mycotic infection, a bacterial infection, a parasitic infection or a viral infection and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of mycotic infections of the torso include tinea versicolor and tinea corporis in or around the torso.

Examples of bacterial infections of the torso include folliculitis, cellulitis and infections associated with minor cuts, scrapes or burns in or around the torso.

Examples of parasitic infection of the torso include leishmaniasis in or around the torso.

Examples of viral infections of the torso include those associated with manifestations of varicella zoster virus in or around the torso.

In one embodiment of the invention, the method of treating tinea versicolor of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating tinea versicolor.

In one embodiment of the invention, the method of treating tinea corporis of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating tinea corporis.

In one embodiment of the invention, the method of treating folliculitis of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating cellulitis of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating cellulitis.

In one embodiment of the invention, the method of treating infections associated with minor cuts, scrapes or burns of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating leishmaniasis of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating leishmaniasis.

In one embodiment of the invention, the method of treating manifestations of varicella zoster virus of the torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating varicella zoster virus.

In one embodiment of the invention, the method of treating tinea versicolor of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating tinea versicolor.

In one embodiment of the invention, the method of treating tinea corporis of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating tinea corporis.

In one embodiment of the invention, the method of treating folliculitis of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating cellulitis of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating cellulitis.

In one embodiment of the invention, the method of treating infections associated with minor cuts, scrapes or burns of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating leishmaniasis of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides to said subject, either locally or systemically, or both. In a preferred embodiment, the active pharmaceutical ingredient is an agent effective in treating leishmaniasis.

In one embodiment of the invention, the method of treating manifestations of varicella zoster virus of the torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating varicella zoster virus.

In one embodiment of the invention, the method of treating tinea versicolor of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating tinea versicolor.

In one embodiment of the invention, the method of treating tinea corporis of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating tinea corporis.

In one embodiment of the invention, the method of treating folliculitis of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating cellulitis of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating cellulitis.

In one embodiment of the invention, the method of treating infections associated with minor cuts, scrapes or burns of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating leishmaniasis of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating leishmaniasis.

In one embodiment of the invention, the method of treating manifestations of varicella zoster virus of the torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating varicella zoster virus.

The present application also relates to a method of treating a dermatological condition of a torso, comprising applying to the intact skin of the torso of a subject in need thereof, a t-shirt, and wherein said t-shirt comprises a dry, non-occlusive, fibrous structure, wherein said t-shirt has been finished with a finishing composition that is a homogeneous dispersion comprising an active pharmaceutical ingredient, a surfactant and a solvent, wherein said dermatological condition comprises either an allergic condition, a cutaneous malignancy, a blister, pruritis, an acneiform eruption, a keratosis, a cutaneous condition secondary to an external cause or a corticosteroid-responsive dermatoses and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of an allergic condition of a torso include urticaria in or around the torso.

Examples of a cutaneous malignancy of a torso include superficial basal cell carcinomas in or around the torso.

Examples of a blisters of a torso include pemphigoid or pemphigus in or around the torso.

Examples of a keratosis of a torso include actinic keratosis in or around the torso.

Examples of a cutaneous condition secondary to an external cause of a torso include post surgical scaring in or around the torso.

In one embodiment of the invention, the method of treating an allergic condition of a torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of a torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating an allergic condition of a torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating urticaria of a torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of a torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating urticaria of a torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is capsaicin, cromolyn sodium, dapsone, doxepin or sulfasalazine.

In one embodiment of the invention, the method of treating pruritis of a torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is a local anesthetic. In another preferred embodiment, the active pharmaceutical ingredient is a topical analgesic drug. In another preferred embodiment, the active pharmaceutical ingredient is cholestyramine, estradiol, isothipendyl, nalfurafine, pseudoephedrine, serlopitant or tradipitant.

In one embodiment of the invention, the method of treating pruritis of a torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is a local anesthetic. In another preferred embodiment, the active pharmaceutical ingredient is a topical analgesic drug. In another preferred embodiment, the active pharmaceutical ingredient is cholestyramine, estradiol, isothipendyl, nalfurafine, pseudoephedrine, serlopitant or tradipitant.

In one embodiment of the invention, the method of treating pruritis of a torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is a local anesthetic. In another preferred embodiment, the active pharmaceutical ingredient is a topical analgesic drug. In another preferred embodiment, the active pharmaceutical ingredient is cholestyramine, estradiol, isothipendyl, nalfurafine, pseudoephedrine, serlopitant or tradipitant.

In one embodiment of the invention, the method of treating a cutaneous malignancy of a torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating a cutaneous malignancy of a torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating a cutaneous malignancy of a torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating superficial basal cell carcinoma of a torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating superficial basal cell carcinoma of a torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating superficial basal cell carcinoma of a torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating blisters of a torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating blisters of a torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating blisters of a torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating pemphigoid of a torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin or tetracycline.

In one embodiment of the invention, the method of treating pemphigoid of a torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin or tetracycline.

In one embodiment of the invention, the method of treating pemphigoid of a torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin or tetracycline.

In one embodiment of the invention, the method of treating pemphigus of a torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating pemphigus of a torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant.

In one embodiment of the invention, the method of treating pemphigus of a torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant.

In one embodiment of the invention, the method of treating a keratosis of a torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of a torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a keratosis of a torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating actinic keratosis of a torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating actinic keratosis of a torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating actinic keratosis of a torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, diclofenac, etretinate, fluorouracil, imiquimod or ingenol.

In one embodiment of the invention, the method of treating a cutaneous condition secondary to an external cause of a torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating a cutaneous condition secondary to an external cause of a torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating a cutaneous condition secondary to an external cause of a torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating post surgical scaring of a torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating post surgical scaring of a torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating post surgical scaring of a torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating a corticosteroid-responsive dermatoses of a torso comprises applying to the intact skin of the torso of a subject in need thereof, a t-shirt, wherein said t-shirt comprises a dry, non-occlusive, fibrous structure, and wherein said t-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of a torso comprises applying to the intact skin of the torso of a subject in need thereof, a short-shirt, wherein said short-shirt comprises a dry, non-occlusive, fibrous structure, and wherein said short-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

In one embodiment of the invention, the method of treating a corticosteroid-responsive dermatoses of a torso comprises applying to the intact skin of the torso of a subject in need thereof, a long-shirt, wherein said long-shirt comprises a dry, non-occlusive, fibrous structure, and wherein said long-shirt has been finished with a finishing composition that is a homogeneous dispersion comprising an active pharmaceutical ingredient, a surfactant and a solvent, and wherein said application is for a time period (e.g., about 1 hour to about 168 hours or about 1 hour to about embodiment, the active pharmaceutical ingredient is a corticosteroid.

The present application also relates to a method of treating a painful condition of the body, comprising applying to the intact skin of the body of a subject in need thereof, a bandage, and wherein said bandage comprises a dry, non-occlusive, fibrous structure, wherein said bandage has been finished with a finishing composition that is a homogeneous dispersion comprising an active pharmaceutical ingredient, a surfactant and a solvent, wherein said painful condition comprises either a superficial nociceptive pain, a deep nociceptive pain, a supra-spinal central neuropathic pain, a spinal central neuropathic pain, a peripheral neuropathic pain or a mixed central and peripheral neuropathic pain and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of superficial nociceptive pain of the body include the pain associated with abrasions, cuts, bruises, burns, sunburns or insect bites.

Examples of deep nociceptive pain of the body include the pain associated with musculoskeletal, post-operative and inflammatory response related pain. Examples of musculoskeletal pain of the body include the pain associated with sprains, tears to ligaments, tendons or muscle tissue, bone fractures, inflammation of tendons, joints, bursae or fascia and degenerative changes to the musculoskeletal system.

Examples of central neuropathic pain of the body include the pain associated with spinal or supra-spinal lesions and compressions to the central nervous system. Examples of the pain of the body associated with supra-spinal lesions to the central nervous system include the pain associated with central venous thrombosis, with a cerebral tumor, with a traumatic brain lesion, with multiple sclerosis, with Parkinson's disease and the pain occurring after a stroke or a thalamotomy. Examples of the pain of the body associated with spinal lesions to the central nervous system include the pain associated with, with a spinal cord injury, with complications of spinal surgery, with ischemic lesions of the spinal cord, with radiation or HIV associated myelopathy.

Examples of peripheral neuropathic pain of the body include the pain associated with lesions and compressions to the peripheral nervous system. Examples of the pain associated with lesions to peripheral nerves of the body include the pain associated with length dependent neuropathy, diabetic neuropathy, lupus neuropathy, Sjogren's syndrome associated neuropathy, hypothyroidism associated neuropathy, HIV related neuropathy, alcoholic neuropathy and inflammatory demyelinating polyradiuloneuropathy. Examples of the pain associated with lesions to nerve roots and posterior ganglia include the pain associated with postherpetic neuralgia and nerve root avulsions. Other examples of peripheral neuropathic pain of the body include the pain associated with paraneoplastic peripheral neuropathy and ganglionopathy, with complications of chemotherapy, radiotherapy and surgery and both Type 1 and Type 2 Complex Regional Pain Syndromes.

In one embodiment of the invention, the method of treating the pain associated with abrasions, cuts, bruises, burns, sunburns and insect bites comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is an anti-histamine. Examples of anti-histamines include one or more of the following: acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

In one embodiment of the invention, the method of treating deep nociceptive pain comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with sprains comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with tears to ligaments, tendons or muscle tissue comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with bone fractures comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with inflammation of tendons, joints, bursae or fascia comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with degenerative changes to the musculoskeletal system comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In yet another preferred embodiment, the active pharmaceutical ingredient is a topical analgesic drug.

In one embodiment of the invention, the method of treating the pain associated with supra-spinal lesions to the central nervous system comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating the pain associated with spinal lesions to the central nervous system comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, baclofen, dronabinol, mexiletine, naloxone or trazodone.

In one embodiment of the invention, the method of treating peripheral neuropathic pain comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

In one embodiment of the invention, the method of treating mixed central and peripheral neuropathic pain comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an NSAID. In another preferred embodiment, the active pharmaceutical ingredient is a topical analgesic drug. In another preferred embodiment, the active pharmaceutical ingredient is an anticonvulsant. In another preferred embodiment, the active pharmaceutical ingredient is amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid or trazodone.

In one embodiment of the invention, the method of treating the pain associated with postherpetic neuralgia comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a local anesthetic. In another preferred embodiment, the active pharmaceutical ingredient is an anticonvulsant. In another preferred embodiment, the active pharmaceutical ingredient is amitriptyline, benfotiamine, capsaicin, citalopram, desipramine, doxepine, duloxetine, escitalopram, fluoxetine, paroxetine, sertraline or thioctic acid.

The present application also relates to a method of treating an inflammatory condition of the body, comprising applying to the intact skin of the body of a subject in need thereof, a bandage, and wherein said bandage comprises a dry, non-occlusive, fibrous structure, wherein said bandage has been finished with a finishing composition that is a homogeneous dispersion comprising an active pharmaceutical ingredient, a surfactant and a solvent, wherein said inflammatory condition comprises either a locally manifesting inflammatory condition or systemic inflammatory condition and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of locally manifesting inflammatory conditions of the body include bursitis, dermatomyositis, lichen planus, neuritis, panniculitis, phlebitis, tendonitis and tendinosis in or around the body.

Examples of systemic inflammatory conditions of the body include autoimmune diseases, gout, manifestations of inflammatory bowel disease, pseudogout, rheumatic fever, sarcoidosis and vasculitis in or around the body. Examples of autoimmune diseases of the body include ankylosing spondylitis, juvenile idiopathic arthritis (JIA), mast cell activation syndrome (MCAS), psoriasis, reactive arthritis, rheumatoid arthritis, Sjögren's syndrome and systemic lupus erythematosus (SLE).

In one embodiment of the invention, the method of treating locally manifesting inflammatory conditions of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is acitretin, chloroquine, colchicine, dapsone, hydroxychloroquine, potassium iodide or sulfasalazine.

In one embodiment of the invention, the method of treating bursitis of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating dermatomyositis of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is chloroquine, dapsone or hydroxychloroquine.

In one embodiment of the invention, the method of treating lichen planus of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a to said subject, either locally or systemically, or both. In a preferred embodiment, the active pharmaceutical ingredient is a corticosteroid. In another preferred embodiment, the active pharmaceutical ingredient is acitretin or sulfasalazine.

In one embodiment of the invention, the method of treating panniculitis of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an NSAID. In another preferred embodiment, the active pharmaceutical ingredient is colchicine or potassium iodide.

In one embodiment of the invention, the method of treating phlebitis of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating tendonitis of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a to said subject, either locally or systemically, or both. In a preferred embodiment, the active pharmaceutical ingredient is an NSAID.

In one embodiment of the invention, the method of treating tendinosis of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an NSAID.

In one embodiment of the invention, the method of treating systemic inflammatory conditions of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an anti-metabolite. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, allopurinol, apremilast, an ACE inhibitor, bexarotene, calcipotriol, coal tar, colchicine, cromolyn sodium, dapsone, etretinate, febuxostat, isotretinoin, minocycline, paricalcitol, pentoxifylline, probenecid, roflumilast, tazarotene or ursodeoxycholic acid.

In one embodiment of the invention, the method of treating autoimmune diseases of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID. In another preferred embodiment, the active pharmaceutical ingredient is acitretin, adapalene, alitretinoin, bexarotene, calcipotriol, coal tar, cromolyn sodium, etretinate, isotretinoin, paricalcitol or tazarotene.

In one embodiment of the invention, the method of treating ankylosing spondylitis of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating juvenile idiopathic arthritis of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a DMARD. In another preferred embodiment, the active pharmaceutical ingredient is an NSAID.

In one embodiment of the invention, the method of treating mast cell activation syndrome of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is an NSAID. In another preferred embodiment, the active pharmaceutical ingredient is cromolyn sodium.

In one embodiment of the invention, the method of treating psoriasis of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is acitretin, adapalene, alitretinoin, bexarotene, calcipotriol, coal tar, etretinate, isotretinoin, paricalcitol, salicylic acid and tazarotene.

In one embodiment of the invention, the method of treating reactive arthritis of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating rheumatoid arthritis of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating Sjögren's syndrome of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID.

In one embodiment of the invention, the method of treating systemic lupus erythematosus of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant. In another preferred embodiment, the active pharmaceutical ingredient is a DMARD. In another preferred embodiment, the active pharmaceutical ingredient is an NSAID.

In one embodiment of the invention, the method of treating gout of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID. In another preferred embodiment, the active pharmaceutical ingredient is allopurinol, colchicine, febuxostat or probenecid.

In one embodiment of the invention, the method of treating manifestations of inflammatory bowel disease of the body comprises applying to the intact skin of the body of a subject in need thereof, a bandage, wherein said bandage comprises a dry, non-occlusive, fibrous structure, and wherein said bandage has been finished with a finishing composition that is a and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is a DMARD.

In one embodiment of the invention, the method of treating pseudogout of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is a DMARD. In another preferred embodiment, the active pharmaceutical ingredient is an NSAID. In another preferred embodiment, the active pharmaceutical ingredient is a colchicine.

In one embodiment of the invention, the method of treating rheumatic fever of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an NSAID.

In one embodiment of the invention, the method of treating sarcoidosis of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-metabolite. In another preferred embodiment, the active pharmaceutical ingredient is a corticosteroid. In another preferred embodiment, the active pharmaceutical ingredient is a DMARD. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is an NSAID. In another preferred embodiment, the active pharmaceutical ingredient is an ACE inhibitor, apremilast, dapsone, minocycline, pentoxifylline, roflumilast or ursodeoxycholic acid.

In one embodiment of the invention, the method of treating vasculitis of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is an immunosuppressant.

The present application also relates to a method of treating an infective condition of the body, comprising applying to the intact skin of the body of a subject in need thereof, a bandage, and wherein said bandage comprises a dry, non-occlusive, fibrous structure, wherein said bandage has been finished with a finishing composition that is a homogeneous dispersion comprising an active pharmaceutical ingredient, a surfactant and a solvent, wherein said infective condition comprises either a mycotic infection, a bacterial infection, a parasitic infection or a viral infection and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of mycotic infections of the body include tinea versicolor and tinea corporis in or around the body.

Examples of bacterial infections of the body include folliculitis, cellulitis, erysipelas, impetigo and infections associated with minor cuts, scrapes or burns in or around the body.

Examples of parasitic infection of the body include leishmaniasis in or around the body.

Examples of viral infections of the body include those associated with manifestations of varicella zoster virus or human papillomavirus in or around the body.

In one embodiment of the invention, the method of treating tinea versicolor of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating tinea versicolor.

In one embodiment of the invention, the method of treating tinea corporis of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating tinea corporis.

In one embodiment of the invention, the method of treating folliculitis of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating folliculitis.

In one embodiment of the invention, the method of treating impetigo of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating impetigo.

In one embodiment of the invention, the method of treating erysipelas of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating erysipelas.

In one embodiment of the invention, the method of treating cellulitis of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating cellulitis.

In one embodiment of the invention, the method of treating infections associated with minor cuts, scrapes or burns of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating infections associated with minor cuts, scrapes or burns.

In one embodiment of the invention, the method of treating leishmaniasis of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating leishmaniasis.

In one embodiment of the invention, the method of treating manifestations of varicella zoster virus of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active embodiment, the active pharmaceutical ingredient is an agent effective in treating varicella zoster virus.

In one embodiment of the invention, the method of treating manifestations of human papillomavirus of the body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an agent effective in treating human papillomavirus.

The present application also relates to a method of treating a dermatological condition of a body, comprising applying to the intact skin of the body of a subject in need thereof, a bandage, and wherein said bandage comprises a dry, non-occlusive, fibrous structure, wherein said bandage has been finished with a finishing composition that is a homogeneous dispersion comprising an active pharmaceutical ingredient, a surfactant and a solvent, wherein said dermatological condition comprises either an allergic condition, a cutaneous malignancy, a blister, pruritis, an acneiform eruption, a keratosis, a cutaneous condition secondary to an external cause or a corticosteroid-responsive dermatoses and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both.

Examples of an allergic condition of a body include atopic dermatitis, contact dermatitis or urticaria in or around the body.

Examples of a cutaneous malignancy of a body include cutaneous t-cell lymphomas, superficial basal cell carcinomas or AIDS-related Kaposi's sarcoma in or around the body.

Examples of a blisters of a body include dermatitis herpetiformis, pemphigoid or pemphigus in or around the body.

Examples of a keratosis of a body include actinic keratosis or hyperkeratosis in or around the body.

Examples of a cutaneous condition secondary to an external cause of a body include post surgical scaring in or around the body.

In one embodiment of the invention, the method of treating an allergic condition of a body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is an anti-leukotriene. In another preferred embodiment, the active pharmaceutical ingredient is a corticosteroid. In another preferred embodiment, the active pharmaceutical ingredient is an immunomodulator. In another preferred embodiment, the active pharmaceutical ingredient is an immunosuppressant. In another preferred embodiment, the active pharmaceutical ingredient is abrocitinib, baricitinib, capsaicin, crisaborole, cromolyn sodium, dapsone, doxepin, desonide, dexpanthenol, ruxolitinib, suplatast or sulfasalazine.

In one embodiment of the invention, the method of treating pruritis of a body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is an anti-histamine. In another preferred embodiment, the active pharmaceutical ingredient is a corticosteroid. In another preferred embodiment, the active pharmaceutical ingredient is a local anesthetic. In another preferred embodiment, the active pharmaceutical ingredient is a topical analgesic drug. In another preferred embodiment, the active pharmaceutical ingredient is cholestyramine, estradiol, isothipendyl, nalfurafine, pseudoephedrine, serlopitant or tradipitant.

In one embodiment of the invention, the method of treating a cutaneous malignancy of a body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active pharmaceutical ingredient is alitretinoin, aminolevulinic acid, bexarotene, carmustine, denileukin diftitox, etoposide, fluorouracil, imiquimod, mechlorethamine, methotrexate, romidepsin, sonidegib, tazarotene, vorinostat or vismodegib.

In one embodiment of the invention, the method of treating blisters of a body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunosuppressant. In another preferred embodiment, the active pharmaceutical ingredient is erythromycin, colchicine, lymecycline, nicotinamide, sulfamethoxypyridazine, sulfapyridine or tetracycline.

In one embodiment of the invention, the method of treating a keratosis of a body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time subject, either locally or systemically, or both. In a preferred embodiment, the active pharmaceutical ingredient is a corticosteroid. In another preferred embodiment, the active pharmaceutical ingredient is acitretin, adapalene, aminolevulinic acid, calcipotriol, diclofenac, etretinate, fluorouracil, imiquimod, ingenol, isotretinoin, lactic acid, maxacalcitol, salicylic acid or tretinoin.

In one embodiment of the invention, the method of treating a cutaneous condition secondary to an external cause of a body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid. In another preferred embodiment, the active ingredient is an immunomodulator. In another preferred embodiment, the active ingredient is an immunosuppressant. In another preferred embodiment, the active ingredient is bleomycin, doxorubicin, fluorouracil, imiquimod, tretinoin or verapamil.

In one embodiment of the invention, the method of treating a corticosteroid-responsive dermatoses of a body comprises applying to the intact skin of the body of a subject in need thereof, a bandage, wherein said bandage comprises a dry, non-occlusive, fibrous structure, and wherein said bandage has been finished with a finishing composition that is a homogeneous dispersion comprising an active pharmaceutical ingredient, a surfactant and a solvent, and wherein said application is for a time period (e.g., about 1 hour to about 168 hours or about 1 hour to about 72 hours) and provides topical administration of a therapeutically effective amount of said active pharmaceutical ingredient to said subject, either locally or systemically, or both. In a preferred embodiment, the active pharmaceutical ingredient is a corticosteroid.

The solvent used in the finishing compositions of the invention can be an aqueous solvent or an organic solvent or a combination of water and an organic solvent. In some aspects, the finishing composition of the invention is an aqueous solution comprising an aqueous solvent combined with the other components. The aqueous solvent can be water. In other aspects, the aqueous solvent can comprise water and an organic, water-miscible solvent. In some aspects, the aqueous solvent comprising at least about 10% (v/v) water. In other aspects, the aqueous solvent comprises at least about 15, 20, 25, 30, 35, 40, 45, 50, 55, 60, 65, 70, 75, 80, 85, 90, 95, or about 100% (v/v) of water. In other aspects, the solvent comprises less than 10% (v/v) water, for example, 9, 8, 7, 6, 5, 4, 3, 2, or 1% (v/v) or less of water. Alternatively, the finishing composition of the invention is an organic solvent (e.g., chloroform, diethyl ether, tetrahydrofuran, toluene, hexanes, methanol, ethanol, propanol, and the like, and mixtures thereof), or combination of organic solvents, combined with other components. In some aspects, the organic solvent can be methanol, ethanol or propanol.

In some aspects, the finishing compositions of the invention are prepared by combining the active pharmaceutical ingredient, or a pharmaceutically acceptable salt thereof, in the solvent to form the finishing composition. The solvent can be water, an organic solvent (e.g., chloroform, diethyl ether, tetrahydrofuran, toluene, hexanes, methanol, ethanol, propanol, and the like, and mixtures thereof), or a mixture of water and an organic solvent, as described herein.

In other aspects, finishing compositions of the invention, in addition to the API (or salt thereof) and the solvent, may further comprise a surfactant. In some of these aspects, the surfactant is combined with the active pharmaceutical ingredient, or a pharmaceutically acceptable salt thereof, in the solvent to form a finishing composition. In some aspects, finishing compositions are prepared by combining a surfactant and the active pharmaceutical ingredient, or a pharmaceutically acceptable salt thereof, in an aqueous solvent to form a finishing composition. In some aspects, finishing compositions are prepared by combining a surfactant and the active pharmaceutical ingredient, or a pharmaceutically acceptable salt thereof, in water to form a finishing composition. In some aspects, finishing compositions are prepared by combining a surfactant and the active pharmaceutical ingredient, or a pharmaceutically acceptable salt thereof, in an organic solvent (e.g., chloroform, diethyl ether, tetrahydrofuran, toluene, hexanes, methanol, ethanol, propanol, and the like, and mixtures thereof) to form a finishing composition. In some aspects, finishing compositions are prepared by combining a surfactant and the active pharmaceutical ingredient, or a pharmaceutically acceptable salt thereof, in solvent comprising water and an organic solvent (e.g., chloroform, diethyl ether, tetrahydrofuran, toluene, hexanes, methanol, ethanol, propanol, and the like, and mixtures thereof) to form a finishing composition.

The surfactant can be present in the finishing composition in an amount of from about 0.1% (w/w) to about 5% (w/w). For example, the surfactant can be present in the finishing composition in an amount of about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or about 5% (w/w). In some aspects, the surfactant can be present in the finishing composition in an amount of from about 0.1% (w/w) to about 3% (w/w) or from about 1% (w/w) to about 3% (w/w). In some aspects, the surfactant can be present in the finishing composition in an amount of from about 0.5% (w/w) to about 3.5% (w/w) or from about 1.5% (w/w) to about 2.5% (w/w). In some aspects, the surfactant can be present in the finishing composition in an amount of from about 1.5% (w/w) to about 4% (w/w).

In other aspects, the surfactant can be present in the finishing composition in an amount of up to 30% (w/w). For example, the surfactant can be present in the finishing composition in an amount of about 6, 7, 8, 9, 10, 11, 12, 13, 14, 15, 16, 17, 18, 19, 20, 21, 22, 23, 24, 25, 26, 27, 28, 29, or about 30% (w/w). In some aspects, the surfactant can be present in the finishing composition in an amount of from about 6% (w/w) to about 30% (w/w) or from about 10% (w/w) to about 30% (w/w). In some aspects, the surfactant can be present in the finishing composition in an amount of from about 15% (w/w) to about 25% (w/w) or from about 15% (w/w) to about 20% (w/w). In some aspects, the surfactant can be present in the finishing composition in an amount of from about 20% (w/w) to about 25% (w/w).

Surfactants suitable for use in the invention are known in the art and include nonionic surfactants. Preferred nonionic surfactants include, for example, fatty acid esters of glycerol and fatty acid esters of sorbitol, ethoxylated amines, fatty acid amide (e.g., polyethoxylated tallow amine, cocamide monoethanolamine, cocamide diethanolamine), terminally blocked ethoxylates (e.g., poloxamers), as well as combinations thereof. In preferred embodiments, the surfactant is a fatty acid ester of sorbitol. Preferred fatty acid esters of sorbitol are polysorbates, for example, polysorbate 20, polysorbate 40, polysorbate 60, and polysorbate 80. Polysorbate 80 is preferred.

The finishing composition can optionally include a humectant. In some embodiments, the finishing composition comprises a humectant and the active pharmaceutical ingredient, or a pharmaceutically acceptable salt thereof, in a solvent (aqueous or organic solvent). In some embodiments, the finishing composition comprises a surfactant, a humectant, and the active pharmaceutical ingredient, or a pharmaceutically acceptable salt thereof, in a solvent (aqueous or organic solvent). In those aspects wherein the humectant is a liquid at ambient temperature, the finishing composition may be prepared in the absence of a solvent. That is, in these embodiments, the finishing compositions comprises a humectant and the active pharmaceutical ingredient, or a pharmaceutically acceptable salt thereof. In other of these aspects, the finishing composition comprises a surfactant, a humectant, and the active pharmaceutical ingredient, or a pharmaceutically acceptable salt thereof.

In those embodiments employing a humectant, the humectant can be present in the finishing composition in an amount of from about 5% (w/w) to about 25% (w/w). For example, the humectant can be present in the finishing composition in an amount of about 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, or about 25% (w/w). In some aspects, the humectant can be present in the finishing composition in an amount of from about 5% (w/w) to about 10% (w/w) or from about 10% (w/w) to about 15% (w/w). In some aspects, the humectant can be present in the finishing composition in an amount of from about 15% (w/w) to about 25% (w/w). In some aspects, the humectant can be present in the finishing composition in an amount of from about 20% (w/w) to about 25% (w/w). In some aspects, the humectant can be present in the finishing composition in an amount of from about 8% (w/w) to about 12% (w/w) or from about 9% (w/w) to about 11% (w/w). In some aspects, the humectant can be present in the finishing composition in an amount of from about 12% (w/w) to about 15% (w/w).

Humectants suitable for use in the invention are known in the art and include, for example, polyalkene glycols, polymeric polyols, sugar alcohols, and combinations thereof. Suitable polyalkene glycols include, for example, polyethylene glycols, such as, for example, polyethylene glycols having an average molecular weight of about 200 daltons to about 800 daltons, for example, 200, 400, 600, or about 800 daltons. Polyethylene glycol 400 is a preferred humectant. Suitable polymeric polyols include, for example, polymers of monosaccharides, such as, for example, polydextrose. Suitable sugar alcohols include, for example, arabitol, erythritol, fucitol, glycerol, galactitol, HSH, iditol, inositol, isomalt, lactitol, maltitol, mannitol, ribitol, sorbitol, threitol, volemitol, and xylitol.

The finishing compositions of the invention can optionally include a permeation enhancer. In those embodiments, the finishing composition comprises a surfactant, a permeation enhancer, and the active pharmaceutical ingredient, or a pharmaceutically acceptable salt thereof, in a solvent (aqueous or organic solvent). In other aspects, the finishing composition comprises a permeation enhancer and the active pharmaceutical ingredient, or a pharmaceutically acceptable salt thereof, in a solvent (aqueous or organic solvent). The finishing composition can optionally include both a humectant and a permeation enhancer. In those embodiments, the finishing composition comprises a surfactant, a humectant, a permeation enhancer, and the active pharmaceutical ingredient, or a pharmaceutically acceptable salt thereof, in a solvent (aqueous or organic solvent). In those aspects wherein the permeation enhancer is a liquid at ambient temperature, the finishing composition may be prepared in the absence of a solvent. That is, in these embodiments, the finishing compositions comprises a surfactant, a permeation enhancer, and the active pharmaceutical ingredient, or a pharmaceutically acceptable salt thereof. In other of these aspects, the finishing composition comprises a surfactant, a humectant, a permeation enhancer, and the active pharmaceutical ingredient, or a pharmaceutically acceptable salt thereof. In other of these aspects, the finishing composition comprises a permeation enhancer and the active pharmaceutical ingredient, or a pharmaceutically acceptable salt thereof.

In those embodiments employing a permeation enhancer, the permeation enhancer can be present in the finishing composition in an amount of from about 5% (w/w) to about 25% (w/w). For example, the permeation enhancer can be present in the finishing composition in an amount of about 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, or about 25% (w/w). In some aspects, the permeation enhancer can be present in the finishing composition in an amount of from about 5% (w/w) to about 10% (w/w) or from about 10% (w/w) to about 15% (w/w). In some aspects, the permeation enhancer can be present in the finishing composition in an amount of from about 15% (w/w) to about 25% (w/w). In some aspects, the permeation enhancer can be present in the finishing composition in an amount of from about 20% (w/w) to about 25% (w/w). In some aspects, the permeation enhancer can be present in the finishing composition in an amount of from about 8% (w/w) to about 12% (w/w) or from about 9% (w/w) to about 11% (w/w). In some aspects, the permeation enhancer can be present in the finishing composition in an amount of from about 12% (w/w) to about 15% (w/w).

Permeation enhancers suitable for use in the invention are known in the art and include, for example, monomeric glycols, monomeric polyols (e.g., glycerol), monomeric alcohols, pyrrolidones, medium chain glycerides, laurate salts, bile salts and derivatives, fatty acids, fatty acid derivatives, chelating agents, sulfoxides, urea and urea derivatives, terpenes, terpenoids, phospholipids, dimethyl acetamide, dimethylformamide, diethylene glycol monoethyl ether, dimethyl isosorbide, and combinations thereof. Suitable monomeric glycols include, for example, propylene glycol. Suitable monomeric alcohols include, for example, ethanol, 2-propanol, and decanol. Suitable pyrrolidones include, for example, 2-pyrrolidone and N-methyl pyrrolidone. Bile salts and derivatives include, for example, sodium glycolate and sodium deoxycholate. Suitable sulfoxides include, for example, dimethyl sulfoxide. Fatty acids are known in the art and include, for example, oleic acid and caprylic acid. Chelating agents include, for example, ethylenediaminetetraacetic acid (EDTA) and citric acid.

The finished, dry, non-occlusive, pharmaceutical fibrous structures of the invention are such that the active pharmaceutical ingredient (or pharmaceutically acceptable salt thereof) is distributed substantially homogenously throughout the fibrous structure. In these aspects, the finish is a “level” finish. That is, the API (or salt thereof) is distributed throughout the fibrous structure such that the concentration of API (or salt thereof) differs by 25% or less, across the whole of the structure. For example, the concentration of API (or salt thereof) differs by 1, 2, 3, 4, 5, 6, 7, 8, 9, 10, 11, 12, 13, 14, 15, 16, 17, 18, 19, 20, 21, 22, 23, 24, or 25% or less across the whole of the fibrous structure.

In some aspects, the finished, dry, non-occlusive, pharmaceutical fibrous structures of the invention are such that the quantitative distribution of API (or salt thereof), the thickness distribution of the API (or salt thereof), the crystallinity of the API (or salt thereof), the adherence of the API (or salt thereof), or a combination thereof, is level throughout the structure. In some aspects, the quantitative distribution of API (or salt thereof) differs by 25% or less, across the whole of the structure. For example, the quantitative distribution of API (or salt thereof) differs by 1, 2, 3, 4, 5, 6, 7, 8, 9, 10, 11, 12, 13, 14, 15, 16, 17, 18, 19, 20, 21, 22, 23, 24, or 25% or less across the whole of the structure. In some aspects, the thickness distribution of API (or salt thereof) differs by 25% or less, across the whole of the structure. For example, the thickness distribution of API (or salt thereof) differs by 1, 2, 3, 4, 5, 6, 7, 8, 9, 10, 11, 12, 13, 14, 15, 16, 17, 18, 19, 20, 21, 22, 23, 24, or 25% or less across the whole of the structure. In some aspects, the crystallinity of the API (or salt thereof) differs by 25% or less, across the whole of the structure. For example, the crystallinity of the API (or salt thereof) differs by 1, 2, 3, 4, 5, 6, 7, 8, 9, 10, 11, 12, 13, 14, 15, 16, 17, 18, 19, 20, 21, 22, 23, 24, or 25% or less across the whole of the structure. In some aspects, the adherence of the API (or salt thereof) differs by 25% or less, across the whole of the structure. For example, the adherence of the API (or salt thereof) differs by 1, 2, 3, 4, 5, 6, 7, 8, 9, 10, 11, 12, 13, 14, 15, 16, 17, 18, 19, 20, 21, 22, 23, 24, or 25% or less across the whole of the structure.

Non-occlusive, finished, dry, pharmaceutical fibrous structures according to the invention (including those produced according to the described methods) contain sufficient quantities of API so as to allow for administration of the API. The amount of API on the finished fibrous structure can be identified by analysis of the mass balance of the finishing composition and/or by the fibrous structure's percentage Add on. The percentage Add on of the finished fibrous structure can be calculated as 100*(weight of the finished fibrous structure−weight of the fibrous structure prior to finishing)/weight of the fibrous structure prior to finishing. In some aspects, the percentage Add on of the finished fibrous structure can be from 20% to about 300%. For example, the percentage Add on of the finished fibrous structure can be 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or 300%, as compared to the dry weight of the fibrous structure absent the finishing composition treatment. In some aspects, the percentage Add on of the finished fibrous structure is increased at least about 20%. In other aspects, the percentage Add on of the finished fibrous structure is increased up to about 300%, as compared to the dry weight of the fibrous structure absent the finishing composition treatment.

In some aspects, the Add on weight is present on the finished fibrous structure of the invention in an amount of about 1 g/m² to about 1200 g/m². In some aspects, the Add on weight is present on the finished fibrous structure of the invention in an amount of about 1 g/m² to about 50 g/m². In some aspects, the Add on weight is present on the finished fibrous structure of the invention in an amount of about 50 g/m² to about 100 g/m². In some aspects, the Add on weight is present on the finished fibrous structure of the invention in an amount of about 100 g/m² to about 150 g/m². In some aspects, the Add on weight is present on the finished fibrous structure of the invention in an amount of about 150 g/m² to about 200 g/m². In some aspects, the Add on weight is present on the finished fibrous structure of the invention in an amount of about 200 g/m² to about 250 g/m². In some aspects, the Add on weight is present on the finished fibrous structure of the invention in an amount of about 250 g/m² to about 300 g/m². In some aspects, the Add on weight is present on the finished fibrous structure of the invention in an amount of about 300 g/m² to about 350 g/m². In some aspects, the Add on weight is present on the finished fibrous structure of the invention in an amount of about 350 g/m² to about 400 g/m². In some aspects, the Add on weight is present on the finished fibrous structure of the invention in an amount of about 400 g/m² to about 450 g/m². In some aspects, the Add on weight is present on the finished fibrous structure of the invention in an amount of about 450 g/m² to about 500 g/m². In some aspects, the Add on weight is present on the finished fibrous structure of the invention in an amount of about 500 g/m² to about 600 g/m². In some aspects, the Add on weight is present on the finished fibrous structure of the invention in an amount of about 600 g/m² to about 700 g/m². In some aspects, the Add on weight is present on the finished fibrous structure of the invention in an amount of about 700 g/m² to about 800 g/m². In some aspects, the Add on weight is present on the finished fibrous structure of the invention in an amount of about 800 g/m² to about 900 g/m². In some aspects, the Add on weight is present on the finished fibrous structure of the invention in an amount of about 900 g/m² to about 1000 g/m². In some aspects, the Add on weight is present on the finished fibrous structure of the invention in an amount of about 1000 g/m² to about 1100 g/m². In some aspects, the Add on weight is present on the finished fibrous structure of the invention in an amount of about 1100 g/m² to about 1200 g/m². In some aspects, the Add on weight is present on the finished fibrous structure of the invention in an amount of about 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1050, 1100, 1150, or about 1200 g/m².

The API finished fibrous structures of the invention are found to maintain both the functional and aesthetic properties of the fibrous structure as found prior to its finishing. As such, the API finished fibrous structures of the invention are found to have minor changes in the tensile strength when compared to the fibrous structure as found prior to its finishing, which do not affect their softness, strength, breathability, vapor transport, flexibility and appearance when compared to their structures prior to finishing. Similarly, the API finished fibrous structures of the invention are found to have minimal friability of the finishing composition from the fibrous structure.

In some aspects, the finished, dry, non-occlusive, pharmaceutical fibrous structures of the invention have a tensile strength of about 10 kgf to about 50 kgf peak load at 25° C. and 51% relative humidity, when tested according to ASTM D5034-09. In some aspects, the finished, dry, non-occlusive, pharmaceutical fibrous structures of the invention have a tensile strength of about 10 kgf to about 15 kgf peak load at 25° C. and 51% relative humidity. In some aspects, the finished, dry, non-occlusive, pharmaceutical fibrous structures of the invention have a tensile strength of about 15 kgf to about 20 kgf peak load at 25° C. and 51% relative humidity. In some aspects, the finished, dry, non-occlusive, pharmaceutical fibrous structures of the invention have a tensile strength of about 20 kgf to about 25 kgf peak load at 25° C. and 51% relative humidity. In some aspects, the finished, dry, non-occlusive, pharmaceutical fibrous structures of the invention have a tensile strength of about 25 kgf to about 30 kgf peak load at 25° C. and 51% relative humidity. In some aspects, the finished, dry, non-occlusive, pharmaceutical fibrous structures of the invention have a tensile strength of about 30 kgf to about 35 kgf peak load at 25° C. and 51% relative humidity. In some aspects, the finished, dry, non-occlusive, pharmaceutical fibrous structures of the invention have a tensile strength of about 35 kgf to about 40 kgf peak load at 25° C. and 51% relative humidity. In some aspects, the finished, dry, non-occlusive, pharmaceutical fibrous structures of the invention have a tensile strength of about 40 kgf to about 45 kgf peak load at 25° C. and 51% relative humidity. In some aspects, the finished, dry, non-occlusive, pharmaceutical fibrous structures of the invention have a tensile strength of about 45 kgf to about 50 kgf peak load at 25° C. and 51% relative humidity. In some aspects, the finished, dry, non-occlusive, pharmaceutical fibrous structures of the invention have a tensile strength of about 10, 15, 20, 25, 30, 35, 40, 45, or about 50 kgf peak load at 25° C. and 51% relative humidity.

In some aspects, the finished, dry, non-occlusive, pharmaceutical fibrous structures of the invention have a tensile strength that is equivalent to, or up to 150% greater than, the tensile strength of the fibrous structure absent the treatment with the finishing composition, when tested according to ASTM D5034-09. In some aspects, the finished, dry, non-occlusive, pharmaceutical fibrous structures of the invention have a tensile strength that is equivalent to the tensile strength of the fibrous structure absent the treatment with the finishing composition. In some aspects, the finished, dry, non-occlusive, pharmaceutical fibrous structures of the invention have a tensile strength that is up to 150% greater than the tensile strength of the fibrous structure absent the treatment with the finishing composition. In some aspects, the finished, dry, non-occlusive, pharmaceutical fibrous structures of the invention have a tensile strength that is 5% to 20% greater than the tensile strength of the fibrous structure absent the treatment with the finishing composition. In some aspects, the finished, dry, non-occlusive, pharmaceutical fibrous structures of the invention have a tensile strength that is 20% to 50% greater than the tensile strength of the fibrous structure absent the treatment with the finishing composition. In some aspects, the finished, dry, non-occlusive, pharmaceutical fibrous structures of the invention have a tensile strength that is 50% to 75% greater than the tensile strength of the fibrous structure absent the treatment with the finishing composition. In some aspects, the finished, dry, non-occlusive, pharmaceutical fibrous structures of the invention have a tensile strength that is 75% to 100% greater than the tensile strength of the fibrous structure absent the treatment with the finishing composition. In some aspects, the finished, dry, non-occlusive, pharmaceutical fibrous structures of the invention have a tensile strength that is 100% to 125% greater than the tensile strength of the fibrous structure absent the treatment with the finishing composition. In some aspects, the finished, dry, non-occlusive, pharmaceutical fibrous structures of the invention have a tensile strength that is 125% to 150% greater than the tensile strength of the fibrous structure absent the treatment with the finishing composition. In some aspects, the finished, dry, non-occlusive, pharmaceutical fibrous structures of the invention have a tensile strength that is 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, or about 150% greater than the tensile strength of the fibrous structure absent the treatment with the finishing composition.

In some aspects, the finished, dry, non-occlusive, pharmaceutical fibrous structures of the invention have a stiffness of about 10 mm to about 50 mm, when tested according to ASTM method D1388-18. In some aspects, the finished, dry, non-occlusive, pharmaceutical fibrous structures of the invention have a stiffness of about 10 mm to about 20 mm. In some aspects, the finished, dry, non-occlusive, pharmaceutical fibrous structures of the invention have a stiffness of about 20 mm to about 30 mm. In some aspects, the finished, dry, non-occlusive, pharmaceutical fibrous structures of the invention have a stiffness of about 30 mm to about 40 mm. In some aspects, the finished, dry, non-occlusive, pharmaceutical fibrous structures of the invention have a stiffness of about 40 mm to about 50 mm. In some aspects, the finished, dry, non-occlusive, pharmaceutical fibrous structures of the invention have a stiffness of about 10, 15, 20, 25, 30, 35, 40, 45, or about 50 mm.

In some aspects, the finished, dry, non-occlusive, pharmaceutical fibrous structures of the invention have a stiffness that is equivalent to, or up to 300% greater than, the stiffness of the fibrous structure absent treatment with the finishing composition, when tested according to ASTM method D1388-18. In some aspects, the finished, dry, non-occlusive, pharmaceutical fibrous structures of the invention have a stiffness that is equivalent to the stiffness of the fibrous structure absent treatment with the finishing composition. In some aspects, the finished, dry, non-occlusive, pharmaceutical fibrous structures of the invention have a stiffness that is up to 300% greater than the stiffness of the fibrous structure absent treatment with the finishing composition. In some aspects, the finished, dry, non-occlusive, pharmaceutical fibrous structures of the invention have a stiffness that is 10% to 50% greater than the stiffness of the fibrous structure absent treatment with the finishing composition. In some aspects, the finished, dry, non-occlusive, pharmaceutical fibrous structures of the invention have a stiffness that is 50% to 100% greater than the stiffness of the fibrous structure absent treatment with the finishing composition. In some aspects, the finished, dry, non-occlusive, pharmaceutical fibrous structures of the invention have a stiffness that is 100% to 150% greater than the stiffness of the fibrous structure absent treatment with the finishing composition. In some aspects, the finished, dry, non-occlusive, pharmaceutical fibrous structures of the invention have a stiffness that is 150% to 200% greater than the stiffness of the fibrous structure absent treatment with the finishing composition. In some aspects, the finished, dry, non-occlusive, pharmaceutical fibrous structures of the invention have a stiffness that is 200% to 250% greater than the stiffness of the fibrous structure absent treatment with the finishing composition. In some aspects, the finished, dry, non-occlusive, pharmaceutical fibrous structures of the invention have a stiffness that is 250% to 300% greater than the stiffness of the fibrous structure absent treatment with the finishing composition. In some aspects, the finished, dry, non-occlusive, pharmaceutical fibrous structures of the invention have a stiffness that is 1, 2, 3, 4, 5, 6, 7, 8, 9, 10, 15, 20, 25, 30, 35, 40, 45, 50, 55, 60, 65, 70, 75, 80, 85 90, 95, 100, 125, 150, 175, 200, 225, 250, 275, or about 300% greater than the stiffness of the fibrous structure absent treatment with the finishing composition.

In some aspects, the finished, dry, non-occlusive, pharmaceutical fibrous structures of the invention have an air permeation rate of about 200 mm/second to about 400 mm/second, when tested according to ASTM D737-09. These air permeation rates are particularly preferred for fibrous structures comprising nylon. In some aspects, the finished, dry, non-occlusive, pharmaceutical fibrous structures of the invention have an air permeation rate of about 200 mm/second to about 250 mm/second. In some aspects, the finished, dry, non-occlusive, pharmaceutical fibrous structures of the invention have an air permeation rate of about 250 mm/second to about 300 mm/second. In some aspects, the finished, dry, non-occlusive, pharmaceutical fibrous structures of the invention have an air permeation rate of about 300 mm/second to about 350 mm/second. In some aspects, the finished, dry, non-occlusive, pharmaceutical fibrous structures of the invention have an air permeation rate of about 350 mm/second to about 400 mm/second. In some aspects, the finished, dry, non-occlusive, pharmaceutical fibrous structures of the invention have an air permeation rate of about 200, 225, 250, 275, 300, 325, 350, 375, or about 400 mm/second.

In some aspects, the finished, dry, non-occlusive, pharmaceutical fibrous structures of the invention have an air permeation rate of about 2100 mm/second to about 2600 mm/second, when tested according to ASTM D737-09. These air permeation rates are particularly preferred for fibrous structures comprising cotton. In some aspects, the finished, dry, non-occlusive, pharmaceutical fibrous structures of the invention have an air permeation rate of about 2100 mm/second to about 2200 mm/second. In some aspects, the finished, dry, non-occlusive, pharmaceutical fibrous structures of the invention have an air permeation rate of about 2200 mm/second to about 2300 mm/second. In some aspects, the finished, dry, non-occlusive, pharmaceutical fibrous structures of the invention have an air permeation rate of about 2300 mm/second to about 2400 mm/second. In some aspects, the finished, dry, non-occlusive, pharmaceutical fibrous structures of the invention have an air permeation rate of about 2400 mm/second to about 2500 mm/second. In some aspects, the finished, dry, non-occlusive, pharmaceutical fibrous structures of the invention have an air permeation rate of about 2500 mm/second to about 2600 mm/second. In some aspects, the finished, dry, non-occlusive, pharmaceutical fibrous structures of the invention have an air permeation rate of about 2100, 2125, 2150, 2175, 2200, 2225, 2250, 2275, 2300, 2325, 2350, 2375, 2400, 2425, 2450, 2475, 2500, 2525, 2550, 2575, or about 2600 mm/second.

In some aspects, the finished, dry, non-occlusive, pharmaceutical fibrous structures of the invention have an air permeation rate that is equivalent to, or +/−50% of, the air permeation rate of the fibrous structure absent treatment with the finishing composition, when tested according to ASTM D737-09. In some aspects, the finished, dry, non-occlusive, pharmaceutical fibrous structures of the invention have an air permeation rate that is equivalent to the air permeation rate of the fibrous structure absent treatment with the finishing composition. In some aspects, the finished, dry, non-occlusive, pharmaceutical fibrous structures of the invention have an air permeation rate that is less than 50% of (−50% of), the air permeation rate of the fibrous structure absent treatment with the finishing composition. In some aspects, the finished, dry, non-occlusive, pharmaceutical fibrous structures of the invention have an air permeation rate that is greater than 50% of (50% of), the air permeation rate of the fibrous structure absent treatment with the finishing composition. In some aspects, the finished, dry, non-occlusive, pharmaceutical fibrous structures of the invention have an air permeation rate that is 50% of (50% of), the air permeation rate of the fibrous structure absent treatment with the finishing composition. In some aspects, the finished, dry, non-occlusive, pharmaceutical fibrous structures of the invention have an air permeation rate that is −50, −45, −40, −35, −30, −25, −20, −15, −10, −5, 0, 5, 10, 15, 20, 25, 30, 35, 40, 45 or about 50% of the air permeation rate of the fibrous structure absent treatment with the finishing composition.

The finishing compositions of the invention can be in the form of a solution, a suspension, an emulsion, or a dispersion. In some aspects, the finishing composition is a solution. In other aspects, the finishing composition is a suspension. In other aspects, the finishing composition is an emulsion. In other aspects, the finishing composition is a dispersion.

According to the invention, the dry, non-occlusive, pharmaceutical fibrous structure finished with a finishing composition of the invention can be used to administer an active pharmaceutical ingredient to a subject. In these methods, the dry, non-occlusive, pharmaceutical fibrous structure finished with a finishing composition of the invention is topically applied to the intact skin of the subject. According to the invention, the dry, non-occlusive, pharmaceutical fibrous structure finished with a finishing composition of the invention is topically applied to the intact skin of the subject for a time sufficient to locally or systemically, or both, administer a therapeutically effective amount of the active pharmaceutical ingredient to the subject. In some aspects, the dry, non-occlusive, pharmaceutical fibrous structure finished with a finishing composition of the invention is topically applied to the intact skin of the subject for a time sufficient to locally administer a therapeutically effective amount of the active pharmaceutical ingredient to the subject. In some aspects, the dry, non-occlusive, pharmaceutical fibrous structure finished with a finishing composition of the invention is topically applied to the intact skin of the subject for a time sufficient to systemically administer a therapeutically effective amount of the active pharmaceutical ingredient to the subject. In some aspects, the dry, non-occlusive, pharmaceutical fibrous structure finished with a finishing composition of the invention is topically applied to the intact skin of the subject for a time sufficient to locally and systemically administer a therapeutically effective amount of the active pharmaceutical ingredient to the subject.

In some aspects, the dry, non-occlusive, pharmaceutical fibrous structure finished with a finishing composition of the invention is topically applied to the subject's intact skin for a time period (e.g., about 1 hour to about 168 hours or about 1 hour to about 72 hours). In some aspects, the dry, non-occlusive, pharmaceutical fibrous structure finished with a finishing composition of the invention is topically applied to the subject's intact skin for a time period of about 8 hours to about 72 hours. In some aspects, the dry, non-occlusive, pharmaceutical fibrous structure finished with a finishing composition of the invention is topically applied to the subject's intact skin for a time period of about 24 hours to about 72 hours. In some aspects, the dry, non-occlusive, pharmaceutical fibrous structure finished with a finishing composition of the invention is topically applied to the subject's intact skin for a time period of about 48 hours to about 72 hours. In some aspects, the dry, non-occlusive, pharmaceutical fibrous structure finished with a finishing composition of the invention is topically applied to the subject's intact skin for a time period of about 12 hours. In some aspects, the dry, non-occlusive, pharmaceutical fibrous structure finished with a finishing composition of the invention is topically applied to the subject's intact skin for a time period of about 1 day. In some aspects, the dry, non-occlusive, pharmaceutical fibrous structure finished with a finishing composition of the invention is topically applied to the subject's intact skin for a time period of about 2 days. In some aspects, the dry, non-occlusive, pharmaceutical fibrous structure finished with a finishing composition of the invention is topically applied to the subject's intact skin for a time period of about 3 days. In some aspects, the dry, non-occlusive, pharmaceutical fibrous structure finished with a finishing composition of the invention is topically applied to the subject's intact skin for a time period of about 4 days. In some aspects, the dry, non-occlusive, pharmaceutical fibrous structure finished with a finishing composition of the invention is topically applied to the subject's intact skin for a time period of about 5 days. In some aspects, the dry, non-occlusive, pharmaceutical fibrous structure finished with a finishing composition of the invention is topically applied to the subject's intact skin for a time period of about 6 days. In some aspects, the dry, non-occlusive, pharmaceutical fibrous structure finished with a finishing composition of the invention is topically applied to the subject's intact skin for a time period of about 7 days.

In some aspects, the therapeutically effective amount of the active pharmaceutical ingredient is administered to the subject throughout the time period that the finished, dry, non-occlusive, pharmaceutical fibrous structure is applied to the subject's intact skin. That is, plasma levels sufficient to treat the subject's condition (either locally, systemically, or both) are substantially consistent throughout the time period. In some aspects, the therapeutically effective amount of the active pharmaceutical ingredient is administered to the subject for about 1 hour to about 72 hours. In some aspects, the therapeutically effective amount of the active pharmaceutical ingredient is administered to the subject for about 8 hours to about 72 hours. In some aspects, the therapeutically effective amount of the active pharmaceutical ingredient is administered to the subject for about 24 hours to about 72 hours. In some aspects, the therapeutically effective amount of the active pharmaceutical ingredient is administered to the subject for about 48 hours to about 72 hours. In some aspects, the therapeutically effective amount of the active pharmaceutical ingredient is administered to the subject for about 1, 2, 3, 4, 5, 6, 7, 8, 9, 10, 11, 12, 13, 14, 15, 16, 17, 18, 19, 20, 21, 22, 23, or about 24 hours. In some aspects, the therapeutically effective amount of the active pharmaceutical ingredient is administered to the subject for at least 4 hours. In some aspects, the therapeutically effective amount of the active pharmaceutical ingredient is administered to the subject for at least 6 hours. In some aspects, the therapeutically effective amount of the active pharmaceutical ingredient is administered to the subject for about 4 days. In some aspects, the therapeutically effective amount of the active pharmaceutical ingredient is administered to the subject for about 5 days. In some aspects, the therapeutically effective amount of the active pharmaceutical ingredient is administered to the subject for about 6 days. In some aspects, the therapeutically effective amount of the active pharmaceutical ingredient is administered to the subject for about 7 days.

In some aspects, the therapeutically effective amount of the active pharmaceutical ingredient is achieved within two hours of applying the finished, dry, non-occlusive pharmaceutical fibrous structure to the subject's intact skin. In some aspects, the therapeutically effective amount of the active pharmaceutical ingredient is achieved within 1 hour of applying the finished, dry, non-occlusive pharmaceutical fibrous structure to the subject's intact skin. In some aspects, the therapeutically effective amount of the active pharmaceutical ingredient is achieved within 30 minutes of applying the finished, dry, non-occlusive pharmaceutical fibrous structure to the subject's intact skin. In some aspects, the therapeutically effective amount of the active pharmaceutical ingredient is achieved within 15 minutes of applying the finished, dry, non-occlusive pharmaceutical fibrous structure to the subject's intact skin. In some aspects, the therapeutically effective amount of the active pharmaceutical ingredient is achieved within 10 minutes of applying the finished, dry, non-occlusive pharmaceutical fibrous structure to the subject's intact skin. In some aspects, the therapeutically effective amount of the active pharmaceutical ingredient is achieved within 5 minutes of applying the finished, dry, non-occlusive pharmaceutical fibrous structure to the subject's intact skin.

The finished, dry, non-occlusive pharmaceutical fibrous structures of the invention, for example, those treated with a finishing composition of the invention, can be applied to the subject's intact skin continuously throughout the time period. For example, in some aspects, the finished, dry, non-occlusive pharmaceutical fibrous structures of the invention, for example, those treated with a finishing composition of the invention, can be applied to the subject's intact skin continuously throughout the daytime hours, for example for about 12 hours to about 18 hours. In other aspects, the finished, dry, non-occlusive pharmaceutical fibrous structures of the invention, for example, those treated with a finishing composition of the invention, can be applied to the subject's intact skin continuously throughout the nighttime hours, for example for about 6 hours to about 12 hours.

In other aspects, the finished, dry, non-occlusive pharmaceutical fibrous structures of the invention, for example, those treated with a finishing composition of the invention, can be applied to the subject's intact skin intermittently throughout the time period. In these embodiments wherein the structure is intermittently applied during the time period, the subject will experience brief periods where the structure is not applied to the subject's intact skin. These periods can range from 1 minute to 1 hour, for example, 1, 2, 3, 4, 5, 6, 7, 8, 9, 10, 11, 12, 13, 14, 15, 20, 25, 30, 35, 40, 45, 50, 55, or about 60 minutes. The subject may experience one or more of these periods during the time period of application. In other aspects, the subject will experience longer periods where the structure is not applied to the subject's intact skin. These periods can range from over to hour to about 12 hours to about 24 hours, for example, 2, 3, 4, 5, 6, 7, 8, 9, 10, 11, 12, 13, 14, 15, 16, 17, 18, 19, 20, 21, 22, 23, or about 24 hours. In some aspects, the structure is applied to the subject's intact skin for about 12 hours, followed by a period of about 12 hours wherein no structure of the invention is applied to the subject's intact skin.

In some aspects, the finished, dry, non-occlusive pharmaceutical fibrous structures of the invention, for example, those treated with a finishing composition of the invention, are replaced with another finished, dry, non-occlusive pharmaceutical fibrous structure of the invention, after the time period. In these aspects, the finished, dry, non-occlusive pharmaceutical fibrous structures of the invention are worn in a manner similar to how conventional garments are worn and are replaced with other garments or finished, dry, non-occlusive pharmaceutical structures of the invention, worn as garments.

In some aspects of the invention, the finished, dry, non-occlusive pharmaceutical fibrous structures are applied to the subject with an intermittent or continuous pressure. In some aspects of the invention, the finished, dry, non-occlusive pharmaceutical fibrous structures are applied to the subject with an intermittent pressure. Intermittent pressure may arise, for example, from the subject's usual activities such as walking, sitting, standing, laying, etc. In some aspects of the invention, the finished, dry, non-occlusive pharmaceutical fibrous structures are applied to the subject with a constant pressure. Constant pressure may arise, for example, from an external, mechanical application of force to the fibrous structure. According to the invention, the amount of active pharmaceutical ingredient administered to the subject from a finished, dry, non-occlusive pharmaceutical fibrous structure of the invention will be clinically equivalent to the amount of active pharmaceutical ingredient administered to the subject absent the pressure. That is, the local and/or systemic levels of the active pharmaceutical agent administered will be independent of the pressure (if any) applied to the fibrous structure during the application to the subject's intact skin.

In some aspects, the subject may produce sweat between the intact skin and the finished, dry, non-occlusive pharmaceutical fibrous structure of the invention. In these embodiments, the amount of active pharmaceutical ingredient administered to the subject will be clinically equivalent to the amount of active pharmaceutical ingredient administered to the subject, absent the sweat.

Methods of producing the finished, dry, non-occlusive, pharmaceutical fibrous structures of the invention are also within the scope of the invention. According to these methods, a fibrous structure (i.e., of synthetic, manmade, and/or natural fibers as described herein) is treated with a finishing composition of the invention. The finishing compositions comprise an active pharmaceutical ingredient (or a pharmaceutically acceptable salt thereof), a surfactant and a solvent. The finishing compositions may optionally include a humectant, a permeation enhancer, or a combination thereof. Finishing compositions of the invention are described in more detail supra. According to the invention, the fibrous structure is treated with the finishing composition for a time sufficient to finish the fibrous structure with the active pharmaceutic ingredient (or salt thereof).

In some aspects, the fibrous structure is treated with the finishing composition for about 5 minutes to about 24 hours. In some aspects, the fibrous structure is treated with the finishing composition for about 5 seconds to about 24 hours. In other aspects, the fibrous structure is treated with the finishing composition for about 5 minutes to about 90 minutes. In other aspects, the fibrous structure is treated with the finishing composition for about 5 minutes to about 60 minutes. In other aspects, the fibrous structure is treated with the finishing composition for about 5 minutes to about 30 minutes. In other aspects, the fibrous structure is treated with the finishing composition for about 5 minutes to about 15 minutes. In other aspects, the fibrous structure is treated with the finishing composition for about 5 minutes to about 10 minutes. In some aspects, the fibrous structure can be treated for about 1, 5, 10, 15, 20, 25, 30, 35, 40, 45, 50, 55, or about 60 minutes. In other embodiments, the fibrous structure can be treated for about 1.5, 2, 2.5, 3, 3.5, 4, 4.5, or about 5 hours. In other embodiments, the fibrous structure can be treated for about 6, 7, 8, 9, 10, 11, 12, 13, 14, 15, 16, 17, 18, 19, 20, 21, 22, 23, or about 24 hours.

The finishing may be a batch process or a continuous process. In a batch process, the fibrous structure is loaded into a process vessel and removed after the process is completed. In a continuous process, the fibrous structure is continuously transported into a finishing bath or a coating device and subsequently to a drying zone.

In some aspects of the invention, a fibrous structure of the invention is treated by immersing the fibrous structure into the finishing composition. The fibrous structure can be immersed for a sufficient amount of time, for example, for about 5 seconds to about 24 hours, preferably about 5 minutes to about 24 hours for the batch process. For example, the fibrous structure can be immersed for about 1, 5, 10, 15, 20, 25, 30, 35, 40, 45, 50, 55, or about 60 minutes. In other embodiments, the fibrous structure can be immersed for about 1.5, 2, 2.5, 3, 3.5, 4, 4.5, or about 5 hours. In other embodiments, the fibrous structure can be immersed for about 6, 7, 8, 9, 10, 11, 12, 13, 14, 15, 16, 17, 18, 19, 20, 21, 22, 23, or about 24 hours.

In other aspects of the invention, a structure of the invention is treated by coating the fibrous structure with the finishing composition. Coating can be achieved using methods known in the art, for example, by spraying or printing. In these methods, the fibrous structure can be treated for a sufficient amount of time, for example, for about 1 second to about 1 hour, preferably about 1 second to about 30 minutes or about 1 second to about 10 minutes. For example, the fibrous structure can be treated for about 1, 5, 10, 15, 20, 25, 30, 35, 40, 45, 50, 55, or about 60 seconds. In other embodiments, the fibrous structure can be treated for about 1, 2, 3, 4, 5, 6, 7, 8, 9, 10, 15, 20, 25, 30, 35, 40, 45, 50, 55, or about 60 minutes.

According to the methods of the invention, the pH of the finishing composition can be, or can be optionally adjusted to, a pH that is, for example, pH 7 or above, during the treatment process. For example, the pH of the finishing composition can be adjusted to 7, 7.1, 7.2, 7.3, 7.4, 7.5, 7.6, 7.8, 7.9, 8, 8.1, 8.2, 8.3, 8.4, 8.5, 8.6, 8.7, 8.8, 8.9, 9, 9.5, or about pH 10 during the treatment process. Such pH adjustment may be desirable when the API is provided in a pharmaceutically acceptable salt form. In such instances, the pH of the finishing composition is adjusted so as to free-base the API. As those of ordinary skill in the art will appreciate, pH can be adjust by adding a sufficient amount of aqueous base (or aqueous buffer) to achieve the desired pH. In preferred embodiments wherein the API is provided as a salt form, pH is adjusted with an appropriate amount of an aqueous solution of KOH or an aqueous solution of NaOH or by an organic base such as diethethanolamine, triethanolamine or diethylamine.

In some embodiments, the pH of the finishing composition is less than pH 7 during the treatment process. For example, the pH of the finishing composition is about 3, 3.5, 4, 4.5, 5, 5.5, 6, or about 6.5 during the treatment process. The pH of the finishing composition can be between about 3 to 7. In other aspects, the pH is from about 5 to 7 during the treatment process. In some aspects, the pH of the finishing composition is adjusted to a pH of 7 or below during the treatment process. Finishing compositions having a pH of 7 or below will be particularly useful for fibrous structures that comprise, or that are, cotton. Typically, a finishing compositions is above pH 7 for fibrous structures that comprise, or that are, polyamide and/or polyester.

According to the invention, the finishing compositions can be heated to a temperature that is about 20° C. during the finishing treatment process. In some aspects, the finishing composition is heated to a temperature of from about 25° C. to about 100° C. For example, the finishing composition can be heated to a temperature of about 25, 30, 35, 40, 45, 50, 55, 60, 65, 70, 75, 80, 85, 90, 95, or about 100° C. In other aspects, the finishing composition is heated to a temperature of from about 30° C. to about 100° C. In other aspects, the finishing composition is heated to a temperature of from about 30° C. to about 60° C. In other aspects, the finishing composition is heated to a temperature of from about 70° C. to about 90° C. In other aspects, the finishing composition is heated to a temperature of from about 40° C. to about 70° C.

In other aspects of the invention, the finishing composition can be cooled during treatment to a temperature that is below 20° C. during the finishing treatment process. In some aspects, the finishing composition be cooled during treatment to a temperature that is between about 0° C. and 20° C. In some aspects, the finishing composition be cooled during treatment to a temperature that is between about 0° C. and 15° C. In some aspects, the finishing composition be cooled during treatment to a temperature that is between about 0° C. and 10° C. In some aspects, the finishing composition be cooled during treatment to a temperature that is about 0, 5, 10, 15, or about 20° C.

After a sufficient time of treatment, either by immersion or otherwise, the finished, non-occlusive, pharmaceutical fibrous structure is removed from the finishing composition. In some aspects, after the fibrous structure is removed from the finishing composition, excess solution can be mechanically removed from the fibrous structure. In those embodiments wherein the finishing compositions comprises a solvent, this process is known as “dewatering” of the finished, non-occlusive pharmaceutical fibrous structure. For example, in these embodiments, the fibrous structure can be spun, for example, by centrifugation, to remove excess finishing composition. In other aspects, the fibrous structure can be hung so as to permit excess finishing composition to drip from the fibrous structure. In other aspects, the fibrous structure can be squeezed so as to permit excess finishing composition be released from the fibrous structure.

In other aspects, for example, following a dewatering step such as a spinning step, mechanical squeezing or directly following removal from the finishing composition, the fibrous structure is dried. As used herein, “drying” refers to the process by which any excess solution and moisture is removed from the fibrous structure. A “dry, finished, non-occlusive, pharmaceutical fibrous structure” is one where excess finishing solution and moisture has been removed from the fibrous structure. The fibrous structure can be dried by any method known in the art, for example, using heat, reduced pressure and heat, vacuum, freeze drying or via sublimation. In some aspects, the fibrous structure is dried at a temperature of from about 150° C. to about 170° C. In other aspects, the fibrous structure is dried at a temperature of from about 90° C. to about 150° C. In other aspects, the fibrous structure is dried at a temperature of from about 30° C. to about 90° C. Such drying conditions, especially secondary to an initial dewatering process, may also be referred to as “curing.”

In some aspects, the finished, non-occlusive, pharmaceutical fibrous structure is rinsed after removal from the finishing composition. The finished, non-occlusive, pharmaceutical fibrous structure can be rinsed with a solvent. For example, in some aspects, the finished, non-occlusive, pharmaceutical fibrous structure is rinsed with water. In other aspects, the finished, non-occlusive, pharmaceutical fibrous structure is rinsed with an organic solvent (e.g., chloroform, diethyl ether, tetrahydrofuran, toluene, hexanes, methanol, ethanol, propanol, and the like, and mixtures thereof). In other aspects, the finished, non-occlusive, pharmaceutical fibrous structure is rinsed with a combination of water and an organic solvent (e.g., chloroform, diethyl ether, tetrahydrofuran, toluene, hexanes, methanol, ethanol, propanol, and the like, and mixtures thereof). In other aspects, the finished, non-occlusive, pharmaceutical fibrous structure is rinsed with both water and an organic solvent, for example, a first rinsing with water and a second rinsing with an organic solvent. In other aspects, the finished, non-occlusive, pharmaceutical fibrous structure is first rinsed with an organic solvent and a subsequent rinse is with water.

In some aspects of the invention, the rate of release of the active pharmaceutical ingredient from the finished, dry, non-occlusive pharmaceutical fibrous structure of the invention can be modified by treating the fibrous structure with a finishing composition of the invention, wherein the temperature of the finishing composition during the treatment is between 5° C. and 95° C. In these aspects, increased temperature within this range decreases the in vitro rate of release of the active pharmaceutical ingredient from the finished, dry, non-occlusive, pharmaceutical fibrous structure after 5 minutes in about 500 mL of aqueous NaOH, pH 13, at room temperature and 450 rpm. In some aspects, the temperature of the finishing composition during the treatment is about 5° C. In some aspects, the temperature of the finishing composition during the treatment is about 25° C. In some aspects, the temperature of the finishing composition during the treatment is about 50° C. In some aspects, the temperature of the finishing composition during the treatment is about 55° C. In some aspects, the temperature of the finishing composition during the treatment is about 60° C. In some aspects, the temperature of the finishing composition during the treatment is about 65° C. In some aspects, the temperature of the finishing composition during the treatment is about 70° C. In some aspects, the temperature of the finishing composition during the treatment is about 75° C. In some aspects, the temperature of the finishing composition during the treatment is about 80° C. In some aspects, the temperature of the finishing composition during the treatment is about 95° C.

In some aspects, the dry, non-occlusive, pharmaceutical fibrous structures finished with a treatment comprising a finishing composition of the invention will exhibit an in vitro release of 85% or less of the active pharmaceutical ingredient after 5 minutes in 500 mL of aqueous NaOH, pH 13, at room temperature and at 450 rpm under sink conditions (i.e., under conditions of sufficient media to ensure unimpaired dissolution). In some aspects, the dry, non-occlusive, pharmaceutical fibrous structures finished with a treatment comprising a finishing composition of the invention will exhibit an in vitro release of 80% or less of the active pharmaceutical ingredient after 5 minutes in 500 mL of aqueous NaOH, pH 13, at room temperature and at 450 rpm. In some aspects, the dry, non-occlusive, pharmaceutical fibrous structures finished with a treatment comprising a finishing composition of the invention will exhibit an in vitro release of 75% or less of the active pharmaceutical ingredient after 5 minutes in 500 mL of aqueous NaOH, pH 13, at room temperature and at 450 rpm. In some aspects, the dry, non-occlusive, pharmaceutical fibrous structures finished with a treatment comprising a finishing composition of the invention will exhibit an in vitro release of 70% or less of the active pharmaceutical ingredient after 5 minutes in 500 mL of aqueous NaOH, pH 13, at room temperature and at 450 rpm. In some aspects, the dry, non-occlusive, pharmaceutical fibrous structures finished with a treatment comprising a finishing composition of the invention will exhibit an in vitro release of 65% or less of the active pharmaceutical ingredient after 5 minutes in 500 mL of aqueous NaOH, pH 13, at room temperature and at 450 rpm. In some aspects, the dry, non-occlusive, pharmaceutical fibrous structures finished with a treatment comprising a finishing composition of the invention will exhibit an in vitro release of 60% or less of the active pharmaceutical ingredient after 5 minutes in 500 mL of aqueous NaOH, pH 13, at room temperature and at 450 rpm. In some aspects, the dry, non-occlusive, pharmaceutical fibrous structures finished with a treatment comprising a finishing composition of the invention will exhibit an in vitro release of 55% or less of the active pharmaceutical ingredient after 5 minutes in 500 mL of aqueous NaOH, pH 13, at room temperature and at 450 rpm. In some aspects, the dry, non-occlusive, pharmaceutical fibrous structures finished with a treatment comprising a finishing composition of the invention will exhibit an in vitro release of 50% or less of the active pharmaceutical ingredient after 5 minutes in 500 mL of aqueous NaOH, pH 13, at room temperature and at 450 rpm.

In some aspects, the dry, non-occlusive, pharmaceutical fibrous structures finished with a treatment comprising a finishing composition of the invention will exhibit an in vitro release of 85% or more of the active pharmaceutical ingredient after 5 minutes in 500 mL of aqueous NaOH, pH 13, at room temperature and at 450 rpm. In some aspects, the dry, non-occlusive, pharmaceutical fibrous structures finished with a treatment comprising a finishing composition of the invention will exhibit an in vitro release of 90% or more of the active pharmaceutical ingredient after 5 minutes in 500 mL of aqueous NaOH, pH 13, at room temperature and at 450 rpm. In some aspects, the dry, non-occlusive, pharmaceutical fibrous structures finished with a treatment comprising a finishing composition of the invention will exhibit an in vitro release of 95% or more of the active pharmaceutical ingredient after 5 minutes in 500 mL of aqueous NaOH, pH 13, at room temperature and at 450 rpm. In some aspects, the dry, non-occlusive, pharmaceutical fibrous structures finished with a treatment comprising a finishing composition of the invention will exhibit an in vitro release of 99% or more of the active pharmaceutical ingredient after 5 minutes in 500 mL of aqueous NaOH, pH 13, at room temperature and at 450 rpm.

Finished, dry, non-occlusive, pharmaceutical fibrous structures produced according to any of the described methods are envisioned. Fibrous structures produced according to the described methods are useful for transdermally administering the APIs. Fibrous structures produced according to the described methods are also useful for dermally administering the APIs. For example, a fibrous structure produced according to a method of the invention can be applied to the skin of a subject in need of treatment with the API. In some aspects, the API is delivered systemically, that is, the API achieves an effective therapeutic concentration in the subject's bloodstream. In other aspects, the API is delivered substantially locally, that is, the API does not achieve an effective therapeutic concentration in the subject's bloodstream, but does achieve an effective therapeutic concentration at the locally-administered site.

“Tensile strength” refers to a stress/strain property of a material and can be measured by a tensile testing machine such as a Testometric® M350 Universal Testing Machine using an accepted standard method such as ASTM D5034-09. Results may be reported in load (kgf) versus elongation (%). “Strength” refers to resistance to break of a material under tensile load and can be measured by a tensile testing machine such as a Testometric® M350 Universal Testing Machine using an accepted standard method such as ASTM D5034-09. Results may be reported in peak load (kgf).

“Softness” or “stiffness” or “flexibility” refers to the ease of deformation and can be measured by a Shirley bending length tester using an accepted standard method such as ASTM method D1388-18. Results may be reported in bending length (mm).

“Breathability” or “air permeability” refers to the ability of a material to allow air flow through it under a given pressure drop and can be measured by an air permeability tester such as YG461E/II Digital Fabric Air Permeability Tester using an accepted standard method such as ASTM D737-09. Results may be reported in air permeability flow rate (mm/sec).

“Vapor transport” refers to the ability of a material to allow moisture vapor flow through it under given conditions and can be measured by a moisture vapor transport rate measuring apparatus such as Ludlow Corp. CS-141 Moisture Transmission Tester using an accepted standard method such as ASTM E96-80. Results may be reported in moisture flow rate (gr/m²/24 hrs) or as the evaporation % weight loss.

“Leveling” refers to the uniformity of finishing in terms of the differing amount of material deposited on different areas of the fibrous structure and can be measured by extraction, color difference spectroscopy or gravimetrically. Segments of the fibrous structure are examined for the local concentrations of the material and results reported in term of relative standard deviation (RSD).

### Aspects

Aspect 1. A finished, non-occlusive, pharmaceutical fibrous structure comprising natural fibers, manmade fibers, synthetic fibers, or a mixture thereof, wherein the fibrous structure has been finished with a finishing composition comprising
   i. an active pharmaceutical ingredient or a pharmaceutically acceptable salt thereof, and a solvent; and
   ii. optionally, a surfactant;
   iii. optionally, a humectant;
   iv. optionally, a permeation enhancer.
Aspect 2. The finished, non-occlusive, pharmaceutical fibrous structure of aspect 1, wherein the finishing composition further comprises a surfactant.
Aspect 3. The finished, non-occlusive, pharmaceutical fibrous structure of aspect 2, wherein the finishing composition comprises the surfactant in an amount of from about 0.1% (w/w) to about 5% (w/w).
Aspect 4. The finished, non-occlusive, pharmaceutical fibrous structure of aspect 2 or aspect 3, wherein the surfactant is a nonionic surfactant.
Aspect 5. The finished, non-occlusive, pharmaceutical fibrous structure of any one of aspects 2 to 4, wherein the surfactant is a fatty acid ester of glycerol, a fatty acid ester of sorbitol, an ethoxylated amine, a fatty acid amide, a terminally blocked ethoxylate, or a combination thereof.
Aspect 6. The finished, non-occlusive, pharmaceutical fibrous structure of any one of aspects 2 to 5, wherein the surfactant is a fatty acid ester of sorbitol, for example, polysorbate 20, polysorbate 40, polysorbate 60, or polysorbate 80, or a combination thereof, preferably polysorbate 80.
Aspect 7. The finished, non-occlusive, pharmaceutical fibrous structure of any one of the preceding aspects, wherein the finishing composition further comprises a humectant.
Aspect 8. The finished, non-occlusive, pharmaceutical fibrous structure of aspect 7, wherein the finishing composition comprises a humectant in an amount of from about 5% (w/w) to about 25% (w/w).
Aspect 9. The finished, non-occlusive, pharmaceutical fibrous structure of aspect 7 or aspect 8, wherein the humectant is a polyalkene glycol, a polymeric polyols, a sugar alcohol, or a combination thereof.
Aspect 10. The finished, non-occlusive, pharmaceutical fibrous structure of any one of aspects 7 to 9, wherein the humectant is a polyalkene glycol, a polymeric polyol, a sugar alcohol, or a combination thereof, preferably a polyethylene glycol, more preferably polyethylene glycol 400.
Aspect 11. The finished, non-occlusive, pharmaceutical fibrous structure of any one of the preceding aspects, wherein the finishing composition further comprises a permeation enhancer.
Aspect 12. The finished, non-occlusive, pharmaceutical fibrous structure of aspect 11, wherein the finishing composition comprises the permeation enhancer in an amount of from about 5% (w/w) to about 15% (w/w).
Aspect 13. The finished, non-occlusive, pharmaceutical fibrous structure of aspect 11 or aspect 12, wherein the permeation enhancer is a monomeric glycol, a monomeric polyol, a monomeric alcohol, a pyrrolidone, a medium chain glyceride, a laurate salt, a bile salt or bile salt derivative, a fatty acid, a fatty acid derivative, a chelating agent, a sulfoxide, a urea or urea derivative, a terpene, a terpenoid, a phospholipid, dimethyl acetamide, dimethylformamide, diethylene glycol monoethyl ether, dimethyl isosorbide, or a combination thereof.
Aspect 14. The finished, non-occlusive, pharmaceutical fibrous structure of any one of the preceding aspects, wherein the finishing composition comprises from about 0.1% (w/w) to about 25% (w/w) of the active pharmaceutical ingredient or the pharmaceutically acceptable salt thereof.
Aspect 15. The finished, non-occlusive, pharmaceutical fibrous structure of any one of the preceding aspects, wherein the active pharmaceutical ingredient is a lipophilic active pharmaceutical ingredient.
Aspect 16. The finished, non-occlusive, pharmaceutical fibrous structure of any one of the preceding aspects, wherein the active pharmaceutical ingredient is distributed substantially homogenously throughout the fibrous structure and the finish is a level finish.
Aspect 17. The finished, non-occlusive, pharmaceutical fibrous structure of aspect 16, wherein the quantitative distribution of the active pharmaceutical ingredient, the thickness distribution of the active pharmaceutical ingredient, the crystallinity of the active pharmaceutical ingredient, the adherence of the active pharmaceutical ingredient, or a combination thereof, is level throughout the fibrous structure.
Aspect 18. The finished, non-occlusive, pharmaceutical fibrous structure of any one of the preceding aspects, wherein the dry weight of the finished fibrous structure is increased from about 20% to about 300% Add-on, as compared to the dry weight of the fibrous structure absent the finishing treatment.
Aspect 19. The finished, non-occlusive, pharmaceutical fibrous structure of any one of aspects 1 to 18, wherein the dry weight of the finished fibrous structure is increased at least about 20% Add-on, as compared to the dry weight of the fibrous structure absent the finishing treatment.
Aspect 20. The finished, non-occlusive, pharmaceutical fibrous structure of any one of aspects 1 to 18, wherein the dry weight of the finished fibrous structure is increased up to about 300% Add-on, as compared to the dry weight of the fibrous structure absent the finishing treatment.
Aspect 21. The finished, non-occlusive, pharmaceutical fibrous structure of any one of the preceding aspects, wherein the Add-on weight is present in an amount of about 1 g/m² to about 1200 g/m².
Aspect 22. The finished, non-occlusive, pharmaceutical fibrous structure of any one of the preceding aspects, in the form of loose fibers, yarn, or fabric.
Aspect 23. The finished, non-occlusive, pharmaceutical fibrous structure of any one of the preceding aspects, in the form of a garment, for example, a sock, a t-shirt, glove, or a band.
Aspect 24. The finished, non-occlusive, pharmaceutical fibrous structure of any one of the preceding aspects, comprising synthetic fibers.
Aspect 25. The finished, non-occlusive, pharmaceutical fibrous structure of any one of the preceding aspects, comprising manmade fibers.
Aspect 26. The finished, non-occlusive, pharmaceutical fibrous structure of any one of the preceding aspects, comprising natural fibers.
Aspect 27. The finished, non-occlusive, pharmaceutical fibrous structure of any one of the preceding aspects, comprising any combination of synthetic, manmade, or natural fibers.
Aspect 28. The finished, non-occlusive, pharmaceutical fibrous structure of any one of the preceding aspects, wherein the structure is non-adhesive to skin.
Aspect 29. The finished, non-occlusive, pharmaceutical fibrous structure of any one of the preceding aspects, wherein the structure is non-irritant.
Aspect 30. The finished, non-occlusive, pharmaceutical fibrous structure of any one of the preceding aspects, wherein the structure is hypoallergenic.
Aspect 31. The finished, non-occlusive, pharmaceutical fibrous structure of any one of the preceding aspects, having a tensile strength of about 10 kgf to about 50 kgf peak load at 25° C. and 51% relative humidity.
Aspect 32. The finished, non-occlusive, pharmaceutical fibrous structure of any one of the preceding aspects, having a tensile strength that is equivalent to, or up to 150% greater than, the tensile strength of the fibrous structure absent the finishing treatment.
Aspect 33. The finished, non-occlusive, pharmaceutical fibrous structure of any one of the preceding aspects, having a stiffness of about 10 mm to about 50 mm.
Aspect 34. The finished, non-occlusive, pharmaceutical fibrous structure of any one of the preceding aspects, having a stiffness that is equivalent to, or up to 300% greater than, the stiffness of the fibrous structure absent the finishing treatment.
Aspect 35. The finished, non-occlusive, pharmaceutical fibrous structure of any one of the preceding aspects, having an air permeation rate of about 200 mm/second to about 400 mm/second.
Aspect 36. The finished, non-occlusive, pharmaceutical fibrous structure of any one of the preceding aspects, having an air permeation rate that is equivalent to, or +/−50% of the air permeation rate of the fibrous structure absent the finishing treatment.
Aspect 37. The finished, non-occlusive, pharmaceutical fibrous structure of any one of the preceding aspects, wherein the solvent is water.
Aspect 38. The finished, non-occlusive, pharmaceutical fibrous structure of any one of aspects 1 to 37, wherein the solvent comprises an organic solvent.
Aspect 39. The finished, non-occlusive, pharmaceutical fibrous structure of any one of aspects 1 to 37, wherein the solvent comprises water and an organic solvent.
Aspect 40. The finished, non-occlusive, pharmaceutical fibrous structure of any one of the preceding aspects, wherein the pH of the finishing composition is adjusted to a pH of 7 or above during the treatment.
Aspect 41. The finished, non-occlusive, pharmaceutical fibrous structure of any one of aspects 1 to 40, wherein the pH of the finishing composition is adjusted to a pH of 7 or below during the treatment.
Aspect 42. The finished, non-occlusive, pharmaceutical fibrous structure of any one of the preceding aspects, wherein the finishing composition is in the form of a solution, a suspension, an emulsion, or a dispersion.
Aspect 43. The finished, non-occlusive, pharmaceutical fibrous structure of any one of the preceding aspects, wherein when the structure is placed in contact with the intact skin of a subject for a time period (e.g., about 1 hour to about 168 hours or about 1 hour to about 72 hours), it provides topical administration of a therapeutically effective amount of said active pharmaceutical ingredient to said subject, either locally or systemically, or both.
Aspect 44. The finished, non-occlusive, pharmaceutical fibrous structure of aspect 43, wherein the therapeutically effective amount is administered for the duration of the time period.
Aspect 45. The finished, non-occlusive, pharmaceutical fibrous structure of any one of aspects 43 to 44, wherein the therapeutically effective amount is administered systemically.
Aspect 46. The finished, non-occlusive, pharmaceutical fibrous structure of any one of aspects 43 to 45, wherein the therapeutically effective amount is administered locally.
Aspect 47. A method of administering an active pharmaceutical ingredient to a subject comprising applying a finished, non-occlusive, pharmaceutical fibrous structure of any one of aspects 1 to 46 to the intact skin of the subject for a time sufficient to locally administer a therapeutically effective amount of the active pharmaceutical ingredient to the subject.
Aspect 48. A method of administering an active pharmaceutical ingredient to a subject comprising applying the finished, non-occlusive, pharmaceutical fibrous structure of any one of aspects 1 to 46 to the intact skin of the subject for a time sufficient to systemically administer a therapeutically effective amount of the active pharmaceutical ingredient to the subject.
Aspect 49. The method of any one of aspects 47 to 48, wherein the finished, non-occlusive, pharmaceutical fibrous structure is applied to the subject's intact skin for a time period that is between about 1 hour and about 72 hours.
Aspect 50. The method of aspect 49, wherein the therapeutically effective amount is administered to the subject throughout the time period.
Aspect 51. The method of aspect 49, wherein the finished, non-occlusive, pharmaceutical fibrous structure is applied to the subject's intact skin continuously throughout the time period.
Aspect 52. The method of aspect 49, wherein the finished, non-occlusive, pharmaceutical fibrous structure is applied to the subject's intact skin intermittently throughout the time period.
Aspect 53. The method of any one of aspects 47 to 52, wherein the therapeutically effective amount is achieved within two hours of applying the finished, non-occlusive, pharmaceutical fibrous structure to the subject's intact skin.
Aspect 54. The method of any one of aspects 47 to 52, wherein the therapeutically effective amount is administered for at least 4 hours.
Aspect 55. The method of any one of aspects 47 to 52, wherein the therapeutically effective amount is administered for at least 6 hours.
Aspect 56. The method of any one of aspects 47 to 52, wherein the application of the structure to the subject comprises an intermittent or continuous pressure.
Aspect 57. The method of aspect 56, wherein the amount of active pharmaceutical ingredient administered to the subject is clinically equivalent to the amount of active pharmaceutical ingredient administered to the subject absent the pressure.
Aspect 58. The method of any one of aspects 47 to 57, wherein the subject's sweat is present between the structure and the subject's intact skin.
Aspect 59. The method of aspect 58, wherein the amount of active pharmaceutical ingredient administered to the subject is clinically equivalent to the amount of active pharmaceutical ingredient administered to the subject absent the sweat.
Aspect 60. A method of producing the finished, non-occlusive, pharmaceutical fibrous structure of any one of aspects 1 to 48 comprising
   a. treating a fibrous structure with a finishing composition comprising
      i. an active pharmaceutical ingredient or a pharmaceutically acceptable salt thereof; and a solvent; and
      ii. optionally, a surfactant;
      iii. optionally, a humectant;
      iv. optionally, a permeation enhancer;
   b. for a time sufficient to finish the fibrous structure with the active pharmaceutical ingredient; and
   c. removing the finished, non-occlusive, pharmaceutical fibrous structure from the finishing composition.
Aspect 61. The method of aspect 60, wherein the solvent is water.
Aspect 62. The method of aspect 60, wherein the solvent comprises an organic solvent.
Aspect 63. The method of aspect 60, wherein the solvent comprises water and an organic solvent.
Aspect 64. The method of any one of aspects 60 to 63, wherein the pH of the finishing composition is adjusted to a pH of 7 or above.
Aspect 65. The method of any one of aspects 60 to 63, wherein the pH of the finishing composition is adjusted to a pH of 7 or below.
Aspect 66. The method of any one of aspects 60 to 63, wherein the finishing composition is in the form of a solution, a suspension, an emulsion, or a dispersion.
Aspect 67. The method of any one of aspects 60 to 63, wherein the finishing composition comprises the surfactant in an amount of from about 0.1% (w/w) to about 5% (w/w).
Aspect 68. The method of aspect 67, wherein the surfactant is a nonionic surfactant, preferably a fatty acid ester of glycerol or a fatty acid ester of sorbitol or a combination thereof, preferably a fatty acid ester of sorbitol, more preferably a polysorbate.
Aspect 69. The method of any one of aspects 60 to 68, wherein the finishing composition comprises a humectant in an amount of from about 5% (w/w) to about 25% (w/w).
Aspect 70. The method of aspect 60, wherein the humectant is a polyalkene glycol, a polymeric polyol, a sugar alcohol, or a combination thereof, preferably a polyethylene glycol, more preferably polyethylene glycol 400.
Aspect 71. The method of any one of aspects 60 to 70, wherein the finishing composition comprises the permeation enhancer in an amount of from about 5% (w/w) to about 15% (w/w).
Aspect 72. The method of aspect 60, wherein the permeation enhancer is a monomeric glycol, a monomeric alcohol, a pyrrolidone, or a combination thereof, preferably a monomeric glycol, more preferably a propylene glycol.
Aspect 73. The method of any one of aspects 60 to 72, wherein the finishing composition comprises from about 0.1% (w/w) to about 25% (w/w) of the active pharmaceutical ingredient or the pharmaceutically acceptable salt thereof.
Aspect 74. The method of any one of aspects 60 to 72, wherein the active pharmaceutical ingredient is a lipophilic active pharmaceutical ingredient.
Aspect 75. The method of any one of aspects 60 to 72, further comprising heating the finishing composition, preferably to a temperature of from about 30° C. to about 100° C., preferably between about 40° C. and about 70° C., prior to treating the fibrous structure.
Aspect 76. The method of any one of aspects 60 to 72, wherein the treating comprises immersing the fibrous structure in the finishing composition or coating the fibrous structure with the finishing composition.
Aspect 77. The method of any one of aspects 60 to 72, wherein the time is from about 5 minutes to about 24 hours, preferably about 5 minutes to about 90 minutes, more preferably about 5 minutes to about 10 minutes.
Aspect 78. The method of any one of aspects 60 to 72, further comprising rinsing the finished, pharmaceutical fibrous structure.
Aspect 79. The method of any one of aspects 60 to 72, further comprising dewatering the finished, pharmaceutical fibrous structure.
Aspect 80. The method of aspect 79, wherein the dewatering is achieved using mechanical means, preferably squeezing, or centrifugation.
Aspect 81. The method of aspect 79, wherein the dewatering is achieved using thermal means.
Aspect 82. A finished, non-occlusive, pharmaceutical fibrous structure produced according to the method of any one of aspects 60 to 81.
Aspect 83. A non-occlusive, pharmaceutical garment comprising the finished, pharmaceutical fibrous structure of any one of aspects 1 to 48.
Aspect 84. A method of controlling the rate of release of active pharmaceutical ingredient from a finished, non-occlusive, pharmaceutical fibrous structure of any one of aspects 1 to 48 comprising
   a. treating a fibrous structure comprising synthetic fibers, manmade fibers, natural fibers, or a combination thereof, with a finishing composition comprising
      i. an active pharmaceutical ingredient or a pharmaceutically acceptable salt thereof; and a solvent; and
      ii. optionally, a surfactant;
      iii. optionally, a humectant;
      iv. optionally, a permeation enhancer;
   b. wherein the treating step is performed with a finishing composition temperature of between 5° C. and 95° C., wherein increased temperature within this range decreases the in vitro rate of release of the active pharmaceutical ingredient from the finished, non-occlusive, pharmaceutical fibrous structure after 5 minutes in about 500 mL aqueous NaOH, pH 13, room temperature, and 450 rpm.
Aspect 85. The method of aspect 84, wherein the finishing composition temperature is about 5° C.
Aspect 86. The method of aspect 84, wherein the finishing composition temperature is about 25° C.
Aspect 87. The method of aspect 84, wherein the finishing composition temperature is about 50° C.
Aspect 88. The method of aspect 84, wherein the finishing composition temperature is about 55° C.
Aspect 89. The method of aspect 84, wherein the finishing composition temperature is about 60° C.
Aspect 90. The method of aspect 84, wherein the finishing composition temperature is about 65° C.
Aspect 91. The method of aspect 84, wherein the finishing composition temperature is about 70° C.
Aspect 92. The method of aspect 84, wherein the finishing composition temperature is about 75° C.
Aspect 93. The method of aspect 84, wherein the finishing composition temperature is about 80° C.
Aspect 94. The method of aspect 84, wherein the finishing composition temperature is about 95° C.
Aspect 95. A non-occlusive, pharmaceutical fibrous structure finished with a treatment comprising a finishing composition comprising an active pharmaceutical ingredient, or a pharmaceutically acceptable salt thereof, which exhibits an in vitro release of 85% or less of the active pharmaceutical ingredient after about 5 minutes in about 500 mL aqueous NaOH, pH 13, room temperature, and at 450 rpm in sink conditions.
Aspect 96. A non-occlusive, pharmaceutical fibrous structure finished with a treatment comprising a finishing composition comprising an active pharmaceutical ingredient, or a pharmaceutically acceptable salt thereof, which exhibits an in vitro release of greater than 85% of the active pharmaceutical ingredient after about 5 minutes in about 500 mL aqueous NaOH, pH 13, room temperature, and at 450 rpm in sink conditions.
Aspect 97. A non-occlusive, pharmaceutical fibrous structure finished with an active pharmaceutical ingredient, or a pharmaceutically acceptable salt thereof.
Aspect 98. The non-occlusive, pharmaceutical fibrous structure of aspect 97, wherein the structure is non-adhesive.
Aspect 99. The non-occlusive, pharmaceutical fibrous structure of aspect 97 or aspect 98, wherein the structure is non-irritant.
Aspect 100. The non-occlusive, pharmaceutical fibrous structure of any one of aspects 97 to aspect 99, wherein the structure is hypoallergenic.
Aspect 101. A method of administering an active pharmaceutical ingredient to a subject comprising topically applying the non-occlusive, pharmaceutical fibrous structure of any one of aspects 97 to 100 to the intact skin of the subject for a time sufficient to locally or systemically, or both, administer a therapeutically effective amount of the active pharmaceutical ingredient to the subject.
Aspect 102. The method of aspect 101, wherein the structure is applied to the subject's intact skin for a time period (e.g., about 1 hour to about 168 hours or about 1 hour to about 72 hours).
Aspect 103. The method of aspect 101 or aspect 102, wherein the therapeutically effective amount of the active pharmaceutical ingredient is administered to the subject throughout the time period.
Aspect 104. A method of treating a condition of a subject comprising applying to the intact skin of a body part of the subject, an item of clothing, wherein the item of clothing comprises:
a dry, non-occlusive, fibrous structure finished with a finishing composition comprising an active pharmaceutical ingredient and a solvent;
wherein the condition is a painful condition, an inflammatory condition, an infective condition, or a dermatological conditions; and
wherein the application is for a time period and provides topical administration of a therapeutically effective amount of the active pharmaceutical ingredient, locally and/or systemically, to the subject.
Aspect 105. The method of aspect 104, wherein the item of clothing is a sock, a knee brace, a shoulder brace, an elbow sleeve, a wrist band, a glove, a neck sleeve, a back brace, a sleeve, a t-shirt, or a bandage.
Aspect 106. The method of aspect 104 or aspect 105, wherein the item of clothing is a sock and the sock is a toed sock, a toeless sock or a non-toed sock.
Aspect 107. The method of aspect 106, wherein the item of clothing is a sock and the sock is an ankle sock, a mid-length sock, a knee-length sock or a high length sock.
Aspect 108. The method of any one of aspects 104 to 107, wherein the item of clothing is a sock and the sock is a toed ankle sock, a toeless ankle sock, a non-toed ankle sock, a toed mid-length sock, a toeless mid-length sock, a non-toed mid-length sock, a toed knee-length sock, a toeless knee-length sock, a non-toed knee-length sock, a toed high-length sock, a toeless high-length sock or a non-toed high-length sock.
Aspect 109. The method of aspect 104 or aspect 105, wherein the item of clothing is a knee brace.
Aspect 110. The method of aspect 104 or aspect 105, wherein the item of clothing is a shoulder brace.
Aspect 111. The method of aspect 104 or aspect 105, wherein the item of clothing is a elbow sleeve.
Aspect 112. The method of aspect 104 or aspect 105, wherein the item of clothing is a wrist band.
Aspect 113. The method of aspect 104 or aspect 105, wherein the item of clothing is a glove and the glove is a short glove or an elbow glove.
Aspect 114. The method of aspect 113, wherein the glove is a finger glove, a fingerless glove, or a mitten.
Aspect 115. The method of aspect 113 or aspect 114, wherein the glove is a finger short glove, finger elbow glove, fingerless short glove, fingerless elbow glove, short glove mitten or elbow glove mitten.
Aspect 116. The method of aspect 104 or aspect 105, wherein the item of clothing is a neck sleeve and the neck sleeve is a closed neck sleeve or a scarf.
Aspect 117. The method of aspect 104 or aspect 105, wherein the item of clothing is a back brace.
Aspect 118. The method of aspect 104 or aspect 105, wherein the item of clothing is a sleeve.
Aspect 119. The method of aspect 104 or aspect 105, wherein the item of clothing is a t-shirt and the t-shirt is a short-shirt or a long shirt.
Aspect 120. The method of aspect 104 or aspect 105, wherein the item of clothing is a bandage.
Aspect 121. The method of any one of aspects 104 to 120, wherein the condition is a painful condition.
Aspect 122. The method of aspect 121, wherein the painful condition is superficial nociceptive pain, deep nociceptive pain, superficial and deep nociceptive pain, central neuropathic pain, peripheral neuropathic pain, or mixed central and peripheral neuropathic pain.
Aspect 123. The method of aspect 121 or aspect 122, wherein the painful condition is superficial nociceptive pain, for example, pain associated with abrasions, cuts, bruises, burns, sunburns, or insect bites.
Aspect 124. The method of aspect 121 or aspect 122, wherein the painful condition is deep nociceptive pain, for example, musculoskeletal pain (e.g., pain associated with sprains, tears to ligaments, tendons or muscle tissue, bone fractures, inflammation of tendons, joints, bursae or fascia and degenerative changes to the musculoskeletal system), post-operative pain, or inflammatory response related pain.
Aspect 125. The method of aspect 121 or aspect 122, wherein the painful condition is central neuropathic pain, for example, pain associated with a central venous thrombosis, pain associated with a cerebral tumor, pain associated with a traumatic brain lesion, pain associated with multiple sclerosis, pain associated with Parkinson's disease, pain associated with the pain occurring after a stroke or a thalamotomy, pain associated with a spinal cord injury, pain associated with complications of spinal surgery, pain associated with ischemic lesions of the spinal cord, pain associated with radiation, or pain associated with HIV associated myelopathy.
Aspect 126. The method of aspect 121 or aspect 122, wherein the painful condition is peripheral neuropathic pain (e.g., pain associated with lesions to peripheral nerves, lesions to nerve roots and posterior ganglia and lesions to cranial nerves), pain associated with compressions to peripheral nerves, pain associated with compressions to nerve plexuses, pain associated with compressions to and nerve roots, pain associated with length dependent neuropathies, pain associated with those lesions caused by systemic disease (e.g., diabetic neuropathy, lupus neuropathy or hypothyroidism associated neuropathy), pain associated with drug-induced disorders (e.g., chemotherapy induced neuropathy, metabolic or nutritional disorders such as alcoholic neuropathy, traumatic and entrapment syndromes), pain associated with post-surgical nerve injuries, pain associated with inflammatory demyelinating polyradiuloneuropathy, pain associated with HIV sensory neuropathy, pain associated with those lesions associated with postherpetic neuralgia and nerve root avulsions (e.g., pain associated with cranial neuralgias such as trigeminal neuralgia), pain associated with paraneoplastic peripheral neuropathy, pain associated with ganglionopathy, pain associated with complications of chemotherapy, pain associated with radiotherapy, pain associated with surgery, pain associated with Type 1 Complex Regional Pain Syndrome, or pain associated with Type 2 Complex Regional Pain Syndrome.
Aspect 127. The method of any one of aspects 104 to 126, wherein the condition is an inflammatory condition.
Aspect 128. The method of aspect 127, wherein the inflammatory condition is a locally manifesting inflammatory condition or a systemic inflammatory condition.
Aspect 129. The method of aspect 127 or aspect 128, wherein the inflammatory condition is bursitis, dermatomyositis, lichen planus, neuritis, panniculitis, phlebitis, tendonitis, or tendinosis.
Aspect 130. The method of aspect 127 or aspect 128, wherein the inflammatory condition is an autoimmune disease (e.g., ankylosing spondylitis, juvenile idiopathic arthritis (JIA), mast cell activation syndrome (MCAS), psoriasis, reactive arthritis, rheumatoid arthritis, Sjögren's syndrome or systemic lupus erythematosus (SLE)), gout, inflammatory bowel disease, pseudogout, rheumatic fever, sarcoidosis, or vasculitis.
Aspect 131. The method of any one of aspects 104 to 130, wherein the condition is an infective condition.
Aspect 132. The method of aspect 131, wherein the infective condition is a mycotic infection (e.g., tinea versicolor, tinea corporis, tinea pedis or onychomycosis), a bacterial infection (e.g., folliculitis, impetigo, erysipelas, cellulitis, or infections associated with minor cuts, scrapes or burns), a parasitic infection (e.g., scabies, leishmaniasis, or hookworm), a viral infection (e.g., a varicella zoster virus infection or a human papillomavirus infection.
Aspect 133. The method of any one of aspects 140 to 132, wherein the condition is a dermatological condition.
Aspect 134. The method of aspect 133, wherein the dermatological condition is an allergic condition (e.g., atopic dermatitis, contact dermatitis or urticaria), a cutaneous malignancy (e.g., cutaneous t-cell lymphomas, superficial basal cell carcinomas or AIDS-related Kaposi's sarcoma), a blister (e.g., dermatitis herpetiformis, pemphigus or pemphigoid), pruitis, acneiform eruption, keratosis (e.g., actinic keratosis or hyperkeratosis), a cutaneous condition secondary to an external cause, or a corticosteroid-responsive dermatoses.
Aspect 135. The method of any one of aspects 104 to 134, wherein the condition is a painful condition and the active pharmaceutical ingredient is articaine, benzocaine, bupivacaine, butamben, cinchocaine, chloroprocaine, cyclomethycaine, dibucaine, dimethisoquin, dyclonine, etidocaine, levobupivacaine, lidocaine, mepivacaine, pramoxine, piperocaine, prilocaine, propoxycaine, procaine, proparacaine, ropivacaine, tetracaine, trimecaine, aceclofenac, aspirin, benzydamine, celecoxib, dexketoprofen, diflunisal, droxicam, dexibuprofen, diclofenac, enolic acid, etodolac, etoricoxib, fenoprofen, firocoxib, flufenamic acid, flurbiprofen, ibuprofen, indomethacin, ketoprofen, ketorolac, lornoxicam, loxoprofen, meclofenamic acid, mefenamic acid, meloxicam, nabumetone, naproxen, nimesulide, oxaprozin, paracetamol, parecoxib, phenylbutazone, piroxicam, salsalate, sulindac, tenoxicam, tolmetin, tolfenamic acid, carbamazepine, gabapentin, lacosamide, lamotrigine, levetiracetam, oxcarbazepine, pregabalin, tiagabine, valproate, vigabatrin, capsaicin, menthol, clonidine, detomidine, dexmedetomidine, duloxetine, medetomidine, brimonidine, guanabenz, camphor, amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid, trazodone or a combination thereof.
Aspect 136. The method of any one of aspects 104 to 134, wherein the condition is an inflammatory condition and the active pharmaceutical ingredient is an anti-histamine (e.g., acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine, or triprolidine), an anti-leukotriene (e.g., curcumin, montelukast, pranlukast, St. John's wort, zafirlukast, or zileuton), an anti-metabolite (e.g., azathioprine, leflunomide, mercaptopurine, methotrexate, mycophenolic acid, pemetrexed or pralatrexate), a corticosteroid (e.g., alclometasone, amcinonide, betamethasone, clobetasol, clocortolone, desonide, desoximetasone, diflorasone, fluocinolone, flurandrenolide, fluticasone, halcinonide, halobetasol, hydrocortisone, prednicarbate, triamcinolone, or mometasone), a disease-modifying antirheumatic drug (DMARD) (e.g., azathioprine, chloroquine, ciclosporin, gold salts, hydroxychloroquine, leflunomide, mesalazine, methotrexate, sulfasalazine or tofacitinib), an immunomodulator (e.g., apremilast, azathioprine, lenalidomide, methotrexate, pentoxifylline, pomalidomide or thalidomide), an immunosuppressant (e.g., azathioprine, cyclophosphamide, cladribine, chlorambucil, cyclophosphamide, cyclosporine, everolimus, mercaptopurine, methotrexate, mycophenolic acid, sirolimus or tacrolimus), a non-steroidal anti-inflammatory drug (NSAID) (e.g., aceclofenac, aspirin, benzydamine, celecoxib, dexketoprofen, diflunisal, droxicam, dexibuprofen, diclofenac, enolic acid, etodolac, etoricoxib, fenoprofen, firocoxib, flufenamic acid, flurbiprofen, ibuprofen, indomethacin, ketoprofen, ketorolac, lornoxicam, loxoprofen, meclofenamic acid, mefenamic acid, meloxicam, nabumetone, naproxen, nimesulide, oxaprozin, paracetamol, parecoxib, phenylbutazone, piroxicam, salsalate, sulindac, tenoxicam, tolmetin, tolfenamic acid, acitretin, adapalene, alitretinoin, allopurinol, apremilast, an ACE inhibitor, bexarotene, calcipotriol, chloroquine, coal tar, colchicine, cromolyn sodium, dapsone, etretinate, febuxostat, hydroxychloroquine, isotretinoin, minocycline, paricalcitol, pentoxifylline, potassium iodide, probenecid, roflumilast, salicylic acid, sulfasalazine, tazarotene or ursodeoxycholic acid or a combination thereof.
Aspect 137. The method of any one of aspects 104 to 134, wherein the condition is an infective condition and the active pharmaceutical ingredient is bacitracin, benzoyl peroxide, cephalexin, ciprofloxacin, clindamycin, dapsone, erythromycin, loxacillin, dicloxacillin, flucloxacillin, linezolid, minocycline, mupirocin, neomycin, polymyxin B, rifampin, trimethoprim-sulfamethoxazole, amoxicillin-clavulanate potassium, bacitracin, cephalexin, chloramphenicol, ciprofloxacin, clindamycin, doxycycline, erythromycin, fusidic acid, gentamicin, levofloxacin, linezolid, mupirocin, neosporin, ofloxacin, ozenoxacin, retapamulin, tetracycline, or trimethoprim-sulphamethoxazole, ampicillin, amoxicillin, amoxicillin-clavulanate potassium, azithromycin, azlocillin, bacampicillin, carbenicillin, cephalexin, clindamycin, cloxacillin, dicloxacillin, epicillin, erythromycin, flucloxacillin, hetacillin, metampicillin, methicillin, mezlocillin, nafcillin, penicillin, piperacillin, pivampicillin, oxacillin, sulbactam, talampicillin, tazobactam, temocillin, ticarcillin, amoxicillin, ampicillin-sulbactam, azithromycin, cefuroxime, cephalexin, ceftriaxone, cefazolin, clarithromycin, clindamycin, dicloxacillin, erythromycin, levofloxacin, nafcillin, oxacillin, penicillin, vancomycin, or bacitracin, chlortetracycline, neomycin, tetracycline, butenafine, selenium, terbinafine, betamethasone, butenafine, ciclopirox, clioquinol, clotrimazole, econazole, haloprogin, luliconazole, miconazole, naftifine, oxiconazole, povidone-iodine, sertaconazole, sulconazole, terbinafine, tolnaftate, undecylenic acid, betamethasone, butenafine, ciclopirox, clioquinol, clotrimazole, econazole, haloprogin, luliconazole, miconazole, naftifine, oxiconazole, povidone-iodine, sertaconazole, sulconazole, terbinafine, tolnaftate, undecylenic acid, benzyl benzoate, crotamiton, ivermectin, lindane, moxidectin, permethrin, precipitated sulfur, salicylic acid, synergized pyrethrins, allopurinol, amphotericin B, antimonite, cotrimoxazole, dapsone, fluconazole, ketoconazole, meglumine, methylbenzethonium chloride, metronidazole, miltefosine, paromomycin, pentoxifylline, raconazole, rifampicin, sodium stibogluconante, albendazole, levamisole, mebendazole, pyrantel pamoate, acyclovir, famciclovir, penciclovir, valacyclovir, benzoyl peroxide, bleomycin, cantharidin, dinitrochlorobenzene, formic acid, imiquimod, podophyllin, salicylic acid, silver nitrate, trichloroacetic acid, zinc oxide, zinc sulfate or a combination thereof.
Aspect 138. The method of any one of aspects 104 to 134, wherein the condition is a dermatological condition and the active pharmaceutical ingredient is an anti-histamine (e.g., acrivastine, azelastine, bepotastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, ketotifen, levocabastine, levocetirizine, loratadine, meclizine, mirtazapine, mizolastine, olopatadine, orphenadrine, phenindamine, pheniramine, phenyltoloxamine, promethazine, pyrilamine, rupatadine, tripelennamine or triprolidine), an anti-leukotriene (e.g., curcumin, montelukast, pranlukast, St. John's wort, zafirlukast or zileuton), a corticosteroid (e.g., alclometasone, amcinonide, betamethasone, clobetasol, clocortolone, desonide, desoximetasone, diflorasone, fluocinolone, flurandrenolide, fluticasone, halcinonide, halobetasol, hydrocortisone, prednicarbate, triamcinolone, or mometasone), an immunomodulator (e.g., apremilast, azathioprine, lenalidomide, methotrexate, pentoxifylline, pomalidomide or thalidomide), an immunosuppressant (e.g., azathioprine, cyclophosphamide, cladribine, chlorambucil, cyclophosphamide, cyclosporine, everolimus, mercaptopurine, methotrexate, mycophenolic acid, pimecrolimus, sirolimus or tacrolimus), a local anesthetic (articaine, benzocaine, bupivacaine, butamben, cinchocaine, chloroprocaine, cyclomethycaine, dibucaine, dimethisoquin, dyclonine, etidocaine, levobupivacaine, lidocaine, mepivacaine, pramoxine, piperocaine, prilocaine, propoxycaine, procaine, proparacaine, ropivacaine, tetracaine or trimecaine), or a topical analgesic drug (e.g, capsaicin, menthol, clonidine, detomidine, dexmedetomidine, duloxetine, medetomidine, brimonidine, guanabenz, camphor, abrocitinib, acitretin, adapalene, alitretinoin, aminolevulinic acid, baricitinib, bexarotene, bleomycin, calcipotriol, capsaicin, carmustine, cholestyramine, colchicine, crisaborole, cromolyn sodium, dapsone, denileukin, desonide, dexpanthenol, diclofenac, diftitox, doxepin, doxorubicin, erythromycin, estradiol, etoposide, etretinate, fluorouracil, imiquimod, ingenol, isothipendyl, isotretinoin, lactic acid, lymecycline, maxacalcitol, mechlorethamine, methotrexate, nalfurafine, nicotinamide, pseudoephedrine, romidepsin, ruxolitinib salicylic acid, serlopitant, sonidegib, sulfamethoxypyridazine, sulfapyridine suplatast, sulfasalazine, tazarotene, tetracycline, tradipitant, tretinoin, verapamil, vorinostat, or vismodegib or a combination thereof.
Aspect 139. The method of any one of aspects 104 to 138, wherein time period is from about 1 hour to about 168 hours.
Aspect 140. The method of aspect 139, wherein the time period is about 1 hour to about 3 hours, 1 hour to about 6 hours, 1 hour to about 8 hours, 1 hour to about 12 hours, 1 hour to about 24 hours, 1 hour to about 48 hours, 1 hour to about 72 hours, 1 hour to about 96 hours, 1 hour to about 120 hours, or 1 hour to about 144 hours.
Aspect 141. The method of any one of aspects 104 to 140, wherein the body part is a foot, a knee, a shoulder, an elbow, a wrist, a hand, neck, back, body appendage, a torso or a combination thereof.
Aspect 142. The method of any one of aspects 104 to 141, wherein the topical administration of the therapeutically effective amount of the active pharmaceutical ingredient is locally administered to the subject.
Aspect 143. The method of any one of aspects 104 to 142, wherein the topical administration of the therapeutically effective amount of the active pharmaceutical ingredient is systemically administered to the subject.
Aspect 144. The method of any one of aspects 104 to 143, wherein the topical administration of the therapeutically effective amount of the active pharmaceutical ingredient is locally and systemically administered to the subject.

The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

Polyethylene glycol 400 (100 g), propylene glycol (100 g), Polysorbate 80 (20 g), and lidocaine HCl (100 g) were added into a beaker. Double distilled water was then added to the beaker, up to final weight of 1000 g. The resulting mixture was stirred and heated to 50° C. to form a clear and homogenous solution. The solution was divided into two aliquots of 500 g, each. A polyamide fibrous structure (˜50 g) was added into each of the aliquots. A 45% w/w potassium hydroxide solution was added to the aliquots, while stirring, until pH reached 7-8 pH values. The aliquots were then placed in a Labomat type <BFA> dying apparatus (Mathis AG, Switzerland) for 30 minutes, at 50° C., while rotating clockwise and anticlockwise. The fibrous structures were removed from the solutions, spun to remove excess solution, and placed into an AEG combined washing-drying machine to dry. Drug content on the finished fibrous structure was estimated by fibrous structure weight gain. Amounts of lidocaine finished on the fibrous structures was further analyzed using HPLC or by UV spectrophotometric techniques.

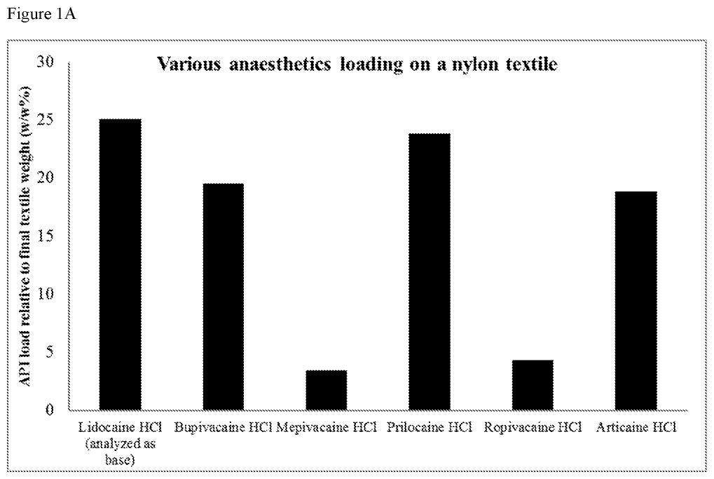

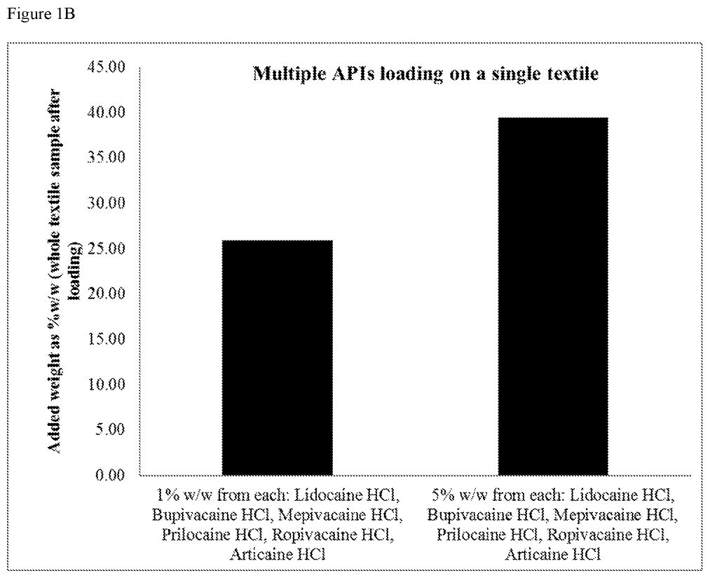

A similar procedure was used to impregnate a polyamide fibrous structure with lidocaine, bupivacaine, mepivacaine, prilocaine, ropivacaine, or articaine, each example using a solution including 10% (w/w) of the API as its HCl salt. See FIG. 1.

A fibrous structure finished with six APIs (lidocaine, bupivacaine, mepivacaine, prilocaine, ropivacaine, and articaine) was prepared using a similar procedure, using a solution including 1% (w/w) or 5% (w/w) of the APIs as their HCl salts. See FIG. 1A.

### Example 2

Lidocaine base was finished on a polyamide fibrous structure using a procedure similar to that described in Example 1, but starting with lidocaine free base and without pH adjustment. Instead of 50° C., the finishing composition was heated to 60° C.

### Example 3

A combination of lidocaine and prilocaine was finished on a polyamide fibrous structure using a procedure similar to that described in Example 1, but with 15% (w/w) of lidocaine HCl and 15% (w/w) of prilocaine HCl.

### Example 4

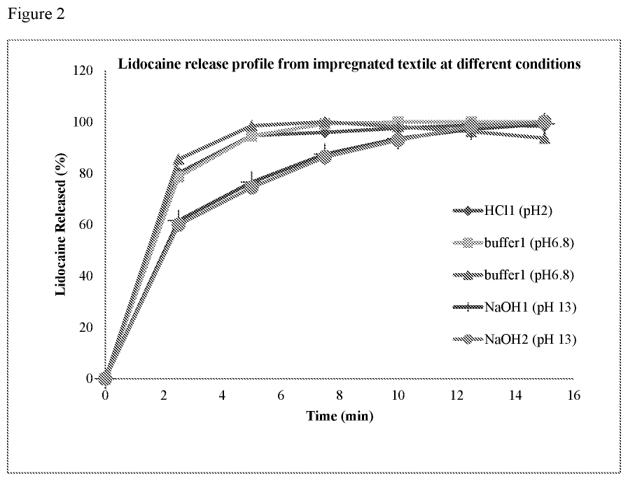

This study was conducted to characterize drug release profiles from a fibrous structure at different pH conditions. 708-DS Dissolution Apparatus 2 (Agilent Technologies Inc., Palo Alto, Calif.) connected to Agilent 8453 Spectrophotometer and UV-visible ChemStation for Dissolution testing were used. Three different pH solutions (pH 2 (n=1), 6.8 (n=2), and 13 (n=2)) were added into separate 900 mL beakers. Substantially identical, lidocaine-loaded, fibrous structures were added and the drug levels in the solutions were measured using an on-line UV spectrophotometer. Results are presented in FIG. 2.

### Example 5

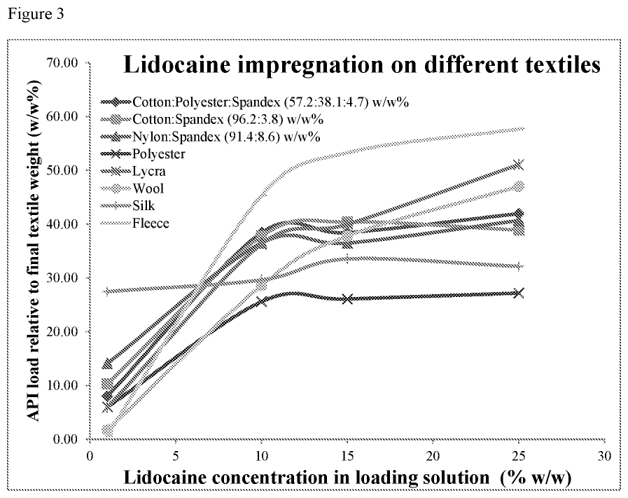

Lidocaine base was finished on different kinds of fibrous structures using a procedure similar to that described in Example 1 at different lidocaine HCl concentrations ranged from 1 to 25 w/w %. (See FIG. 3)

### Example 6

Skin levels and plasma pharmacokinetic profiles of lidocaine after application of an finished fibrous structure with lidocaine in a mini pig model were tested. A commercially available 5% lidocaine patch was used as a reference (Lidoderm®, Endo pharmaceuticals, Inc, Chadds Ford, Calif.). Two lidocaine patches (total of 1400 mg lidocaine; 280 cm² surface area) were compared with finished fibrous structures containing ˜3000 mg lidocaine HCl in each prototype; 280 cm² surface area. See Table 1.

TABLE 1

| | | | Propylene | | |
|---|---|---|---|---|---|
| Examples | Lidocaine* | PEG400** | glycol** | Polysorbate80** | Sorbitol** |
| LS-12 | about 3000 mg | | | 2% (w/w) | |
| LS-13 | about 3000 mg | | 10% (w/w) | 2% (w/w) | |
| LS-14 | about 3000 mg | 10% (w/w) | 10% (w/w) | 2% (w/w) | |
| LS-15 | about 3000 mg | | 10% (w/w) | 2% (w/w) | 10% (w/w) |
| LS-16 | about 5000 mg | 10% (w/w) | 10% (w/w) | 2% (w/w) | |

*lidocaine content on finished fibrous structure, as measured by HPLC
**content in finishing composition

Test articles were placed on the dorsal skin of the tested animals. Test and reference article patches were applied to cover 280 cm² of the application site. Prior to application of the reference patch or the test articles, dose sites were gently wiped with tap water-moistened paper tissues/towels or with gauze pads. The application site was completely dry before application of the patches. No soaps or any cleansing agents were used to clean the application site. During the dose application, gloves were changed between each animal treatment by the study-performing technicians.

In general, each reference patch (LIDODERM® (lidocaine patch)) was 140 square centimeters in area (containing 700 mg lidocaine) and had one piece of release liner that covered the entire adhesive layer. The sticky side of two patches was applied to the pig's skin. To ensure the patch reference article was in good contact with the application site, the patch was gently smoothed out after application to ensure no air bubbles had been entrapped under the surface of the patch, and then gently pressed around the edges for at least 30 seconds. Two reference article patches were applied to the width of the application site, directly adjacent to each other, and were covered with a gauze pad. To ensure fixation, the gauze pad was secured with Vet-Flex.

The test article was a fibrous structure finished with Lidocaine of 280 square centimeter area. The test article was attached with pins and adhesive tape to Velcro strips that could wrap around the animal's abdomen in order to affix the test article. The region of the fibrous structure finished with Lidocaine was positioned over the application site (mid-abdomen just lateral of the spine on the left side).

The test article was held in contact with the skin by applying adhesive tape (3M) on the edges of the test fibrous structure and secured with Vet Flex tape but without covering the portion of the fibrous structure laden with Lidocaine.

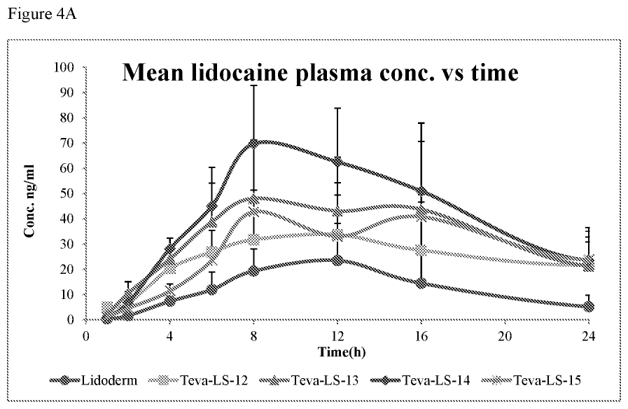

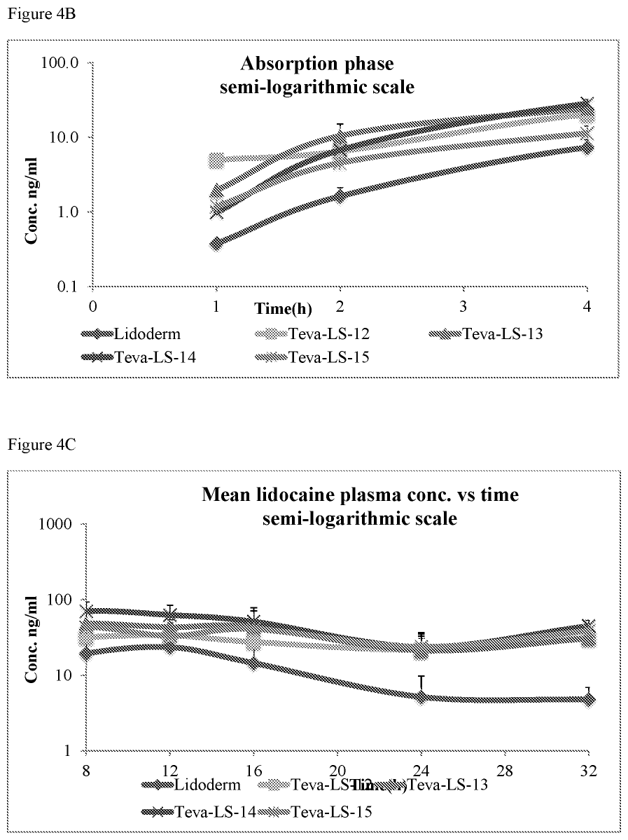

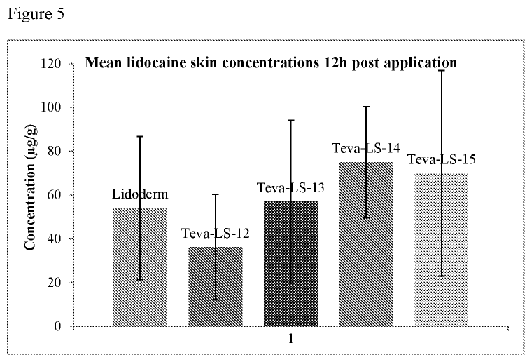

The administration period for the reference/test articles was 12 h. Skin biopsies were collected after 12 hrs. Plasma samples were taken up to 32 hours post application. Lidocaine levels in skin biopsies and in plasma were analyzed. See Table 2, below. Results are depicted in FIGS. 4, 4A, 4B, and 5.

TABLE 2

| | Duration and | | | | Skin |
|---|---|---|---|---|---|
| | Area of | Dose and | Plasma | Skin | local |
| Treatment | Application | Drug Load | Collection | Collection | tolerance |
| LIDODERM ® | 12 h | 1400 mg | 0, 1, 2, 4, 6, 8, | SC, full | Draze |
| 2 patches | 280 cm² | 5 mg/cm² | 10, 12, 16, 14, | thickness skin | modified test |
| Teva-LS-12 | (7.5% BSA) | 3000 mg | and 32 h | at 12 h | at 12 h |
| Teva-LS-13 | | 11 mg/cm² | | | |
| Teva-LS-14 | | | | | |
| Teva-LS-15 | | | | | |

Results - Mean Plasma Concentrations vs. Time

### Example 7

Local pressure and human sweat effects on LS-16 prototype (see Table 1) were investigated. Skin samples as well as blood samples for investigating the plasma pharmacokinetic profiles of lidocaine after application of lidocaine-finished fibrous structure in Gottingen minipigs were collected. An finished fibrous structure containing 5000 mg lidocaine was applied to each animal; test article surface area was 280 cm². Individual test article doses were administered on a fixed basis (1 test article/animal) via topical application. At least 1 day prior to the day of dosing, an area of dorsal skin larger than, but including the application site, was clipped to become free of hair and to allow uniform application of doses and clear observation of the application site. Prior to application of the test articles, dose sites were gently wiped with tap water-moistened gauze pads. The application site was completely dry before application of the test articles. No soaps or any cleansing agents were used to clean the application site.

The test articles were supplied as a fibrous structure finished with Lidocaine of 280 cm², attached with pins and adhesive tape to Velcro strips that wrapped the animal around its abdomen in order to affix the test article. The region of the fibrous structure finished with Lidocaine was positioned over the application site perpendicular to the spine. The test article was held in contact with the skin by applying adhesive tape on the edges of the test fibrous structure and secured with Vet Flex tape without covering the portion of the fibrous structure laden with Lidocaine.

The test article was applied for approximately 12 hours. At the end of the application period, the dressing was removed and skin irritation examination was performed using the modified Draize scoring system. For each group, the dose site was scored/graded predose and at 12 hours postdose (immediately following patch removal).

Evaluation of Skin Reactions Using Draize Scoring System
Erythema (Redness) and Eschar (Scabbing) Formation

| | |
|---|---|
| 0 | Normal |
| 1 | Very slight erythema (barely perceptible) |
| 2 | Well-defined erythema |
| 3 | Moderate-to-severe erythema |
| 4 | Severe erythema (beet redness) to slight |
| | eschar formation (injuries in depth) |

Edema (Swelling) Formation

| | |
|---|---|
| 0 | Normal |
| 1 | Very slight edema (barely perceptible) |
| 2 | Slight edema (edges of area well defined by definite raising) |
| 3 | Moderate edema (area raised approximately 1 mm) |
| 4 | Severe edema (raised more than 1 mm and |
| | extending beyond area of exposure) |

This study showed that mild scoring for erythema noted score of “2” for one minipig treated with LIDODERM. Other animals were scored “0” or “1” for erythema. All animals were scored “0” for edema and eschar. After test articles removal, skin was cleaned followed by skin sample collection process. Skin stripping and skin biopsy samples were taken.

### Pressure Application:

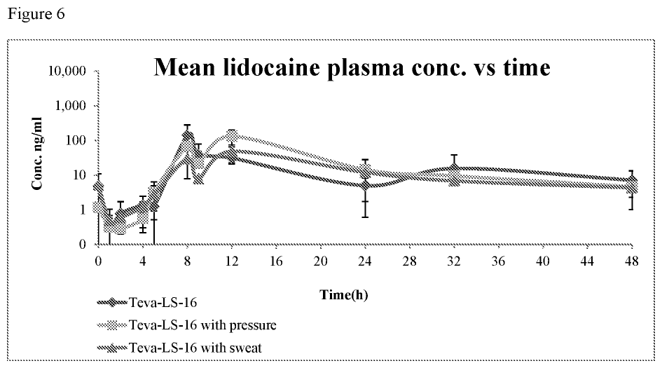

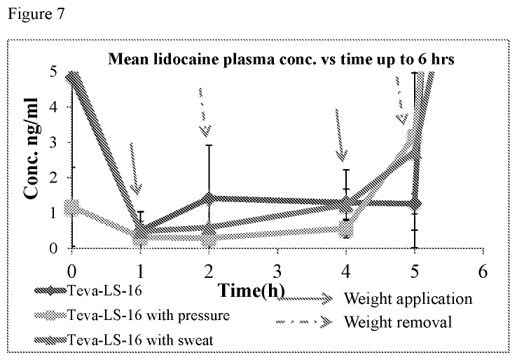

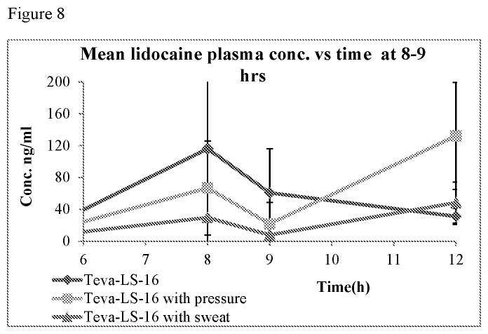

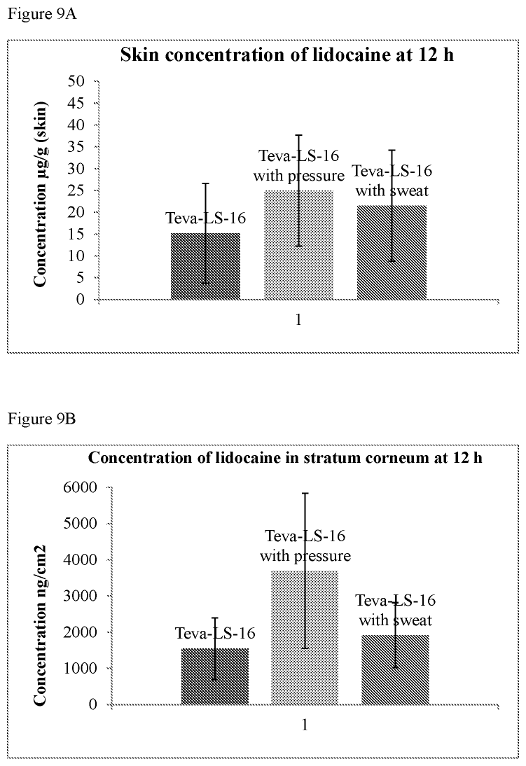

Pressure was applied to the dose application area on 3 separate occasions for duration of approximately 1 hour each time using a sand weight 0.75 kg. The weight was applied at the following time ranges relative to the time of the dose application: 1-2 hours postdose, 4-5 hours postdose and 8-9 hours postdose. The weight was applied across the test article perpendicular to the spine. See FIGS. 6, 7, 8, 9A, and 9B.

### Human Sweat Application:

Normal Sweat Human Fluid from individual donors was purchased from mybiosource (San Diego, Calif.) and was sprayed on the test article at approximately 1 and 4 hours after the dose application. At each occasion of human sweat application approximately 1.5 g of the human sweat was uniformly sprayed over the test article (approx. 280 cm²). The test article was covered with occlusive dressing. The dressing was removed after approximately 1 hr. See FIGS. 6, 7, 8, 9A, and 9B.

### Example 8

The study design, including group designations and dose levels, is shown in Table 3.

TABLE 3

| | No. of | | No. of Test | No. of | Duration of | Target Dose Level |
|---|---|---|---|---|---|---|
| Group | Animals | Treatment | Articles/dose | Doses | each dose | (mg/animal) |
| 1 | 3 | LIDODERM ® | 2^{a} | 4^{a} | 24 h | 1400 |
| 2 | 5 | Teva-LS-16 | 1^{b} | 4^{b} | 24 h | 5000 |

Note:
^{a}Two patches were applied approximately every 24 hours for 96 hours (total of 4 applications).
^{b}One test article was applied approximately every 24 hours for 96 hours (total of 4 applications).

Evaluation of local tolerance and PK of lidocaine following a continuous application (every 24 hours) of 4 doses of fibrous structure relative to LIDODERM® in mini-pigs (n=3-5) was conducted. The treatment duration was 96 hours. The tested products were replaced every 24 hours. Endpoints were plasma PK, skin PK, skin local tolerance Draize test. Reference articles (LIDODERM® patch) were administered as 2 patches/animal/application via topical application. Individual Teva-LS-16 test article doses were administered on a fixed basis 1 test article/animal/application via topical application.

At least 1 day prior to the day of dosing, an area of dorsal skin larger than, but including the application site, was clipped free of hair to allow uniform application of doses and clear observation of the application site. Prior to application of the test article, dose sites were gently wiped with tap water-moistened gauze pads. The application site was completely dry before application of the patches. No soaps or any cleansing agents were used to clean the application site. The test article used was a fibrous structure finished with Lidocaine of 280 cm² area and attached with pins and adhesive tape to Velcro strips that used to wrap the animal around its abdomen in order to affix the test article. The region of the fibrous structure finished with Lidocaine was positioned over the application site perpendicular to the spine. The test article was held in contact with the skin by applying adhesive tape on the edges of the test fibrous structure and secured with Vet Flex tape that did not cover the portion of the fibrous structure laden with the drug. The test articles were applied approximately every 24 hours up to 96 hours (Groups 1 and 2, total of 4 applications).

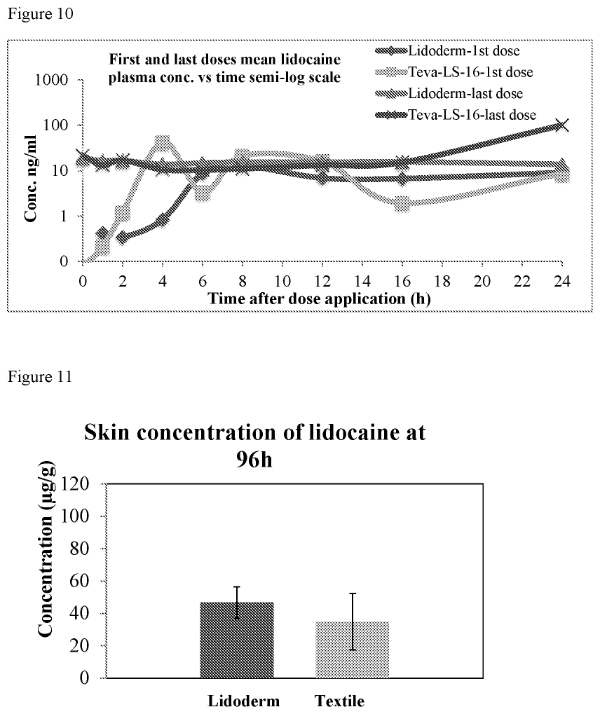

At the end of the application period, the test/reference articles were removed; the application sites were scored, and then gently wiped with gauze pads. After cleaning, skin samples were collected (skin stripping and skin biopsies). Results are depicted in FIGS. 10 and 11.

### Example 9: Study of Different Temperatures and API Concentrations on Fibrous Structure Loading

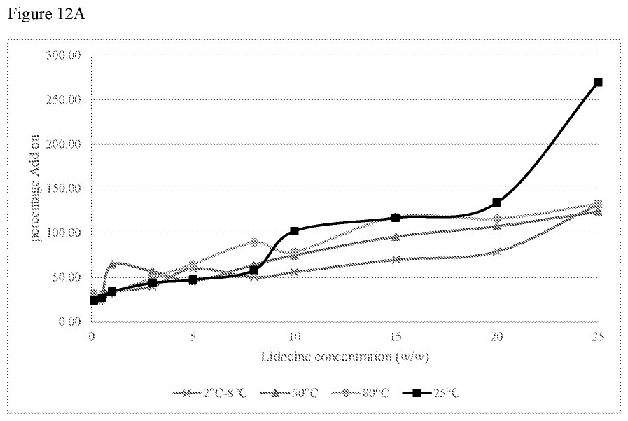

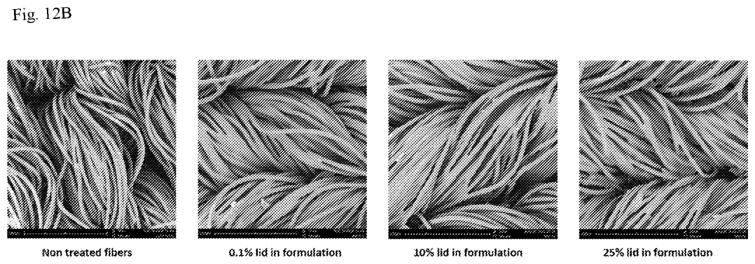

10 g aqueous compositions of 10% PEG 400, 10% propylene glycol and 2% Polysorbate 80 together with lidocaine at concentrations between 0.1% to 25% were added to 20 ml vessels and placed on controlled temperature, 400 RPM, stirrer plates at either 2-8° C., room temperature, 50° C. or 80° C. After reaching the required temperature, 0.5 g samples of 91.4% polyamide/8.6% elastane were added to the vessels and mixed for a further hour. The samples were then removed from their vessels, weighed and left to dry overnight at room temperature before their dry weight and percentage Add on were determined. The results are shown in Table 4 and FIGS. 12 and 12A.

TABLE 4

| Lidocaine | 2-8° C. | RT | 50° C. | 80° C. |
|---|---|---|---|---|
| % (w/w) | % Add on | % Add on | % Add on | % Add on |
| 0.1 | 25.93 | 23.91 | 32.73 | 32.20 |
| 0.5 | 31.48 | 26.79 | 29.79 | 23.91 |
| 1 | 34.00 | 33.96 | 64.44 | 32.14 |
| 3 | 40.00 | 43.75 | 56.52 | 48.98 |
| 5 | 60.00 | 47.27 | 46.00 | 64.91 |
| 8 | 50.00 | 58.00 | 64.15 | 89.13 |
| 10 | 55.77 | 102.04 | 74.55 | 78.72 |
| 15 | 69.77 | 116.98 | 95.74 | 117.78 |
| 20 | 78.72 | 134.04 | 107.41 | 116.00 |
| 25 | 132.08 | 269.57 | 124.00 | 132.56 |

### Example 10. Study of Squeezing and Washing on Fibrous Structure Loading

100 ml aqueous compositions of 10% PEG400, 10% propylene glycol and 2% Polysorbate 80 together with lidocaine at concentrations of 0.1% or 25% were added to 250 ml vessels and placed on controlled temperature stirrer plates and heated to either 25° C. or 80° C. Once the temperature had been reached, 6 samples of 91.4% polyamide/8.6% elastane were added to each vessel. After a further hour, samples were then either a) left to dry at room temperature, b) squeezed—the sample was placed between the 2 layers of blotting paper and pressed for 2 min by the flat plate with a weight of 2.2 kg and then allowed to dry at room temperature, c) washed—using 200 ml water, the sample was mixed for 1 min in solution using a 400 RPM magnetic bar stirrer and then allowed to dry at room temperature or d) washed in 0.1M NaOH—the sample was mixed for 1 minute in 200 ml 0.1M NaOH solution that had been cooled to about 17° C. using a 400 RPM magnetic bar stirrer and then allowed to dry at room temperature. After drying, the sample's dry weight and percentage Add on were determined. The results are shown in Table 5.

TABLE 5

| | % Add on | | | | |
|---|---|---|---|---|---|
| | lidocaine | dried | | washed | washed |
| temperature | concentration | at RT | squeezed | in water | in NaOH |
| 25° C. | 0.1 | 42.31 | 7.23 | 1.40 | 2.74 |
| | 25 | 118.56 | 43.01 | 3.02 | 7.05 |
| 80° C. | 0.1 | 53.21 | 6.30 | 2.02 | 2.97 |
| | 25 | 136.85 | 112.83 | 83.44 | 55.28 |

The lidocaine concentration in the mother liquor was assayed for the 0.1% samples using HPLC (Luna C18(2), 100 Å, 5 μm, 250×4.6 mm (Phenomenex), mobile phase—water with acetic acid (pH 3.4) (80%) and Acetonitrile (20%), UV detection at 254 nm at room temperature, flow rate of 1.5 mL/min) and found to be 0.995 mg/ml for the 0.1%/25° C. liquor and 0.862 mg/ml for the 0.1%/80° C. liquor indicating that approximately 0.5 mg (0.5%) of lidocaine was loaded from the 0.1%/25° C. sample and 13.8 mg (14%) from the 0.1%/80° C. sample.

### Example 11. Study of API without Excipients on Fibrous Structure Loading

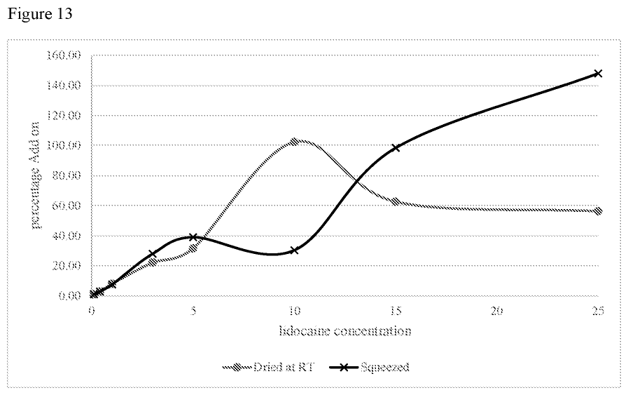

Aqueous compositions of 200 g of lidocaine at concentrations between 0.1% and 25% without additional excipients were added to were added to 500 ml vessels and placed in a Mathis Labomat, warmed to 60° C. and rotated at 40 PRM. Once the temperature was achieved, samples of 91.4% polyamide/8.6% elastane were added to each vessel. After a further hour, samples were then either a) left to dry at room temperature for 14 hours or b) squeezed—the sample was placed between the 2 layers of blotting paper and pressed for 2 min by the flat plate with a weight of 2.2 kg and then allowed to dry at room temperature for 14 hours. After drying, the sample's dry weight and percentage Add on were determined. The results are shown in Table 6 and FIG. 13.

TABLE 6

| Lidocaine | Dried at RT | Squeezed |
|---|---|---|
| [% w/w] | % Add on | % Add on |
| 0.1 | 1.03 | 1.09 |
| 0.4 | 2.86 | 2.91 |
| 1 | 8.03 | 7.57 |
| 3 | 22.17 | 28.01 |
| 5 | 31.61 | 39.06 |
| 10 | 102.39 | 30.39 |
| 15 | 62.68 | 98.46 |
| 25 | 56.33 | 148.02 |

The lidocaine concentration in the mother liquor was assayed for the 0.1, 0.4 and 1% samples using HPLC (Luna C18(2), 100 Å, 5 nm, 250×4.6 mm (Phenomenex), mobile phase—water with acetic acid (pH 3.4) (80%) and Acetonitrile (20%), UV detection at 254 nm at room temperature, flow rate of 1.5 mL/min) and found to be 0.457 mg/ml for the 0.1% liquor, 1.489 mg/ml for the 0.4% liquor and 2.659 mg/ml for the 1%° C. liquor indicating that approximately 108.6 mg (54%) of lidocaine was loaded from the 0.1% sample, 502.2 mg (63%) from the 0.4% sample and 1468 mg (73%) from the 1% sample.

### Example 12. Study of Humectants on Fibrous Structure Loading

Aqueous compositions of 7 different humectants at concentrations of 5%, 15% and 25% with, or without, lidocaine and 2% Polysorbate 80, were added to 20 ml vessels and placed on controlled temperature 400 RPM stirrer plates and warmed to 60° C. After 20 minutes of stirring, a 0.4 g sample of 91.4% polyamide/8.6% elastane was added to each vessel. After a further hour, the samples were removed from their vessels, squeezed, weighed and left to dry at room temperature for a further 46 hours before their dry weight and percentage Add on were determined. The results are shown in Table 7.

TABLE 7

| Humectant | % Humectant | % Lidocaine | % PS80 | % Add on |
|---|---|---|---|---|
| Mannitol | 5 | 10 | 2 | 37.170 |
| | 15 | 10 | 2 | 52.217 |
| | 25 | 10 | 2 | 72.422 |
| | 25 | 0 | 2 | 39.535 |
| Galen IQ | 5 | 10 | 2 | 37.890 |
| Isomalt | 15 | 10 | 2 | 33.647 |
| | 25 | 10 | 2 | 68.766 |
| | 25 | 0 | 2 | 47.002 |
| Sorbitol | 5 | 10 | 2 | 47.573 |
| | 15 | 10 | 2 | 59.352 |
| | 25 | 10 | 2 | 73.934 |
| | 25 | 0 | 2 | 58.072 |
| Propanediol | 5 | 10 | 2 | 37.713 |
| | 15 | 10 | 2 | 47.087 |
| | 25 | 10 | 2 | 65.187 |
| | 25 | 0 | 2 | 30.162 |
| PEG 200 | 5 | 10 | 2 | 40.865 |
| | 15 | 10 | 2 | 65.060 |
| | 25 | 10 | 2 | 79.612 |
| | 25 | 0 | 2 | 37.717 |
| PEG 400 | 5 | 10 | 2 | 34.653 |
| | 15 | 10 | 2 | 69.424 |
| | 25 | 10 | 2 | 75.000 |
| | 25 | 0 | 2 | 34.343 |
| PEG 600 | 5 | 10 | 2 | 42.748 |
| | 15 | 10 | 2 | 55.025 |
| | 25 | 10 | 2 | 59.512 |
| | 25 | 0 | 2 | 39.423 |

### Example 13. Study of Different Permeation Enhancers on Fibrous Structure Loading

Aqueous compositions of 11 different permeation enhancers at concentrations of 5%, 15% and 25% with, or without, lidocaine and 2% Polysorbate 80, were added to 20 ml vessels and placed on controlled temperature 400 RPM stirrer plates, warmed to 60° C. and divided into 2 groups of compositions. For Group 1 compositions, after 40 minutes of stirring, a 0.4 g sample of 91.4% polyamide/8.6% elastane was added to each vessel. After a further hour, the samples were removed from their vessels, squeezed, weighed and left to dry at room temperature for a further 16 hours before their dry weight and percentage Add on were determined. For Group 2 compositions, after 45 minutes of stirring, a 0.4 g sample of 90% polyamide/10% elastane was added to each vessel. After 2 hours, the samples were removed from their vessels, squeezed, weighed and left to dry at room temperature for a further 66 hours before their dry weight and percentage Add on were determined. The results for Group 1 compositions are shown in Table 8 and for Group 2 compositions in Table 9.

TABLE 8

| Permeation | % Permeation | | | |
|---|---|---|---|---|
| enhancer | enhancer | % Lidocaine | % PS80 | % Add on |
| Propylene | 5 | 10 | 2 | 38.261 |
| glycol | 15 | 10 | 2 | 44.975 |
| | 25 | 10 | 2 | 59.502 |
| | 25 | 0 | 2 | 31.494 |
| Isopropyl | 5 | 10 | 2 | 31.982 |
| Myristate | 15 | 10 | 2 | 47.532 |
| | 25 | 10 | 2 | 50.229 |
| | 25 | 0 | 2 | 59.529 |
| 2-pyrrolidone 5 | 5 | 10 | 2 | 36.343 |
| carboxylic acid | 15 | 10 | 2 | 53.616 |
| | 25 | 10 | 2 | 65.848 |
| | 25 | 0 | 2 | 94.761 |
| EDTA | 5 | 10 | 2 | 58.701 |
| | 15 | 10 | 2 | 65.049 |
| | 25 | 10 | 2 | 118.590 |
| | 25 | 0 | 2 | 80.918 |
| Citric acid | 5 | 10 | 2 | 24.557 |
| | 15 | 10 | 2 | 38.780 |
| | 25 | 10 | 2 | 53.012 |
| | 25 | 0 | 2 | 52.163 |
| Ethanol | 5 | 10 | 2 | 33.333 |
| | 15 | 10 | 2 | 38.902 |
| | 25 | 10 | 2 | 29.754 |
| | 25 | 0 | 2 | 9.534 |
| N-Methyl-2- | 5 | 10 | 2 | 35.135 |
| pyrrolidone | 15 | 10 | 2 | 39.303 |
| | 25 | 10 | 2 | 60.287 |
| | 25 | 0 | 2 | 31.417 |

TABLE 9

| Permeation | % Permeation | | | |
|---|---|---|---|---|
| enhancer | enhancer | % Lidocaine | % PS80 | % Add on |
| 2-propanol | 5 | 10 | 2 | 37.084 |
| | 15 | 10 | 2 | 35.469 |
| | 25 | 10 | 2 | 22.628 |
| | 25 | 0 | 2 | 5.470 |
| Glycerol | 5 | 10 | 2 | 44.301 |
| | 15 | 10 | 2 | 55.000 |
| | 25 | 10 | 2 | 80.000 |
| | 25 | 0 | 2 | 51.891 |
| Erythritol | 5 | 10 | 2 | 40.909 |
| | 15 | 10 | 2 | 51.163 |
| | 25 | 10 | 2 | 85.610 |
| | 25 | 0 | 2 | 40.464 |

### Example 14. Study of Different Surfactants on Fibrous Structure Loading

Aqueous compositions of 9 different surfactants at concentrations of 0.1%, 5% and 10% with, or without, lidocaine, were added to 20 ml vessels and placed on controlled temperature 400 RPM stirrer plates and warmed to 60° C. After 2 hours of stirring, a 0.4 g sample of 91.4% polyamide/8.6% elastane was added to each vessel. After a further hour, the samples were removed from their vessels, squeezed, weighed and left to dry at room temperature for a further 66 hours before their dry weight and percentage Add on were determined. The results are shown in Table 10.

TABLE 10

| Surfactant | % Surfactant | % Lidocaine | % Add on |
|---|---|---|---|
| Glyceryl mono | 0.1/0.1 | 10 | 92.386 |
| isostearate/ | 5/5 | 10 | 58.186 |
| Polysorbate 80 | 10/10 | 10 | 53.283 |
| | 10/10 | 0 | 61.369 |
| Polysorbate 20 | 0.1 | 10 | 56.934 |
| | 5 | 10 | 72.372 |
| | 10 | 10 | 82.282 |
| | 10 | 0 | 21.945 |
| Polysorbate 40 | 0.1 | 10 | 53.465 |
| | 5 | 10 | 43.000 |
| | 10 | 10 | 54.722 |
| | 10 | 0 | 18.500 |
| Polysorbate 80 | 0.1 | 10 | 67.143 |
| | 5 | 10 | 44.340 |
| | 10 | 10 | 54.306 |
| | 10 | 0 | 19.656 |
| Poloxamer 124 NF | 0.1 | 10 | 62.857 |
| | 5 | 10 | 49.409 |
| | 10 | 10 | 79.853 |
| | 10 | 0 | 21.957 |
| Poloxamer 188 NF | 0.1 | 10 | 45.278 |
| | 5 | 10 | 63.659 |
| | 10 | 10 | 57.627 |
| | 10 | 0 | 17.848 |
| Poloxamer 127 NF | 0.1 | 10 | 54.613 |
| | 5 | 10 | 36.186 |
| | 10 | 10 | 67.482 |
| | 10 | 0 | 18.780 |
| Cremophor RH40 | 0.1 | 10 | 58.621 |
| | 5 | 10 | 37.037 |
| | 10 | 10 | 46.384 |
| | 10 | 0 | 20.581 |
| Kolliphor ELP 35 | 0.1 | 10 | 79.028 |
| | 5 | 10 | 38.308 |
| | 10 | 10 | 47.585 |
| | 10 | 0 | 19.643 |

### Example 15: Study of Different Solvents on Fibrous Structure Loading

A 10 g aqueous (70%) ethanol (30%) composition of 10% PEG 400, 10% propylene glycol and 2% Polysorbate 80 together with 10% lidocaine was added to 20 ml vessel and placed on a controlled temperature 400 RPM stirrer plate and warmed to 50° C. After reaching the required temperature, a 0.5 g sample of 91.4% polyamide/8.6% elastane was added to the vessel and mixed for a further hour. The sample was then removed from its vessels weighed and left to dry overnight at room temperature before its dry weight and percentage Add on were determined (percentage Add on—56%).

### Example 16: Study of Diclofenac Fibrous Structure Loading

10 g aqueous compositions of 10% Diclofenac sodium salt, 10% PEG 400, 10% propylene glycol and 2% Polysorbate 80 with, or without 20% aqueous ethanol or NaOH 5N, were added to 20 ml vessels and placed on controlled temperature, 400 RPM, stirrer plates and warmed to 60° C. After reaching the required temperature, 0.5 g samples of 91.4% polyamide/8.6% elastane were added to the vessels and mixed for a further hour. The samples were then removed from their vessels, squeezed, weighed and left to dry overnight at room temperature before their dry weight and percentage Add on were determined. The results are shown in Table 11.

TABLE 11

| Concentration | | | |
|---|---|---|---|
| ethanol % (w/w) | NaOH | pH | % Add on |
| — | — | 8.5 | 42.2 |
| 13.6 | — | 8.3 | 44.1 |
| — | 1 drop | 13.2 | 32.0 |
| — | 1 drop | 14.0 | 46.0 |

The diclofenac concentration in the mother liquor was assayed for both samples not containing NaOH using HPLC (Luna C18(2), 100 Å, 5 μm, 250×4.6 mm (Phenomenex), mobile phase—40% water with acetic acid (pH 3.4) and 60% Acetonitrile, UV detection at 210 nm at room temperature, flow rate of 1.5 mL/min) and found to be 93.68 mg/g for the water based liquor and 90.76 mg/g for the ethanol based liquor indicating that approximately 91.4 mg (17%) of diclofenac was loaded from the water based sample and 104.7 mg (18%) from the ethanol based sample.

### Example 17: Study of Clotrimazole Fibrous Structure Loading

200 g compositions of either a) 10% Clotrimazole, 20% PEG400, 15% propylene glycol, 5% Polysorbate 80 in 50% aqueous ethanol or b) 10% Clotrimazole, 20% PEG400, 15% propylene glycol, 5% Polysorbate 80 in ethanol were added to 500 ml vessels and placed in a Mathis Labomat, warmed to 60° C. and rotated at 40 PRM. Once the temperature was achieved, 10 g samples of 91.4% polyamide/8.6% elastane were added to each vessel. After a further hour, the samples were then either squeezed and then allowed to dry at room temperature for overnight. After drying, the sample's dry weight and percentage Add on were determined and the mean Clotrimazole content on the sample was calculated for each of the samples. The results are shown in Table 12.

TABLE 12

| | | | Calculated |
|---|---|---|---|
| | Mean | | Clotrimazole |
| Composition | % Add on | RSD | % content |
| a | 65 | 7.29 | 35 |
| b | 46 | 1.16 | 25 |

### Example 18: Study of Ibuprofen Fibrous Structure Loading

200 g compositions of either a) 10% Ibuprofen, 10% PEG400, 10% propylene glycol, 2% Polysorbate 80 in 30% aqueous ethanol or b) 10% Ibuprofen, 10% PEG400, 10% propylene glycol, 2% Polysorbate 80 in ethanol were added to 500 ml vessels and placed in a Mathis Labomat, warmed to 60° C. and rotated at 40 PRM. Once the temperature was achieved, 10 g samples of 91.4% polyamide/8.6% elastane were added to each vessel. After a further hour, the samples were then either squeezed and then allowed to dry at room temperature for overnight. After drying, the sample's dry weight and percentage Add on were determined and the mean Ibuprofen content on the sample was calculated for each of the samples. The results are shown in Table 13.

TABLE 13

| | | | Calculated |
|---|---|---|---|
| | Mean | | Ibuprofen |
| Composition | % Add on | RSD | % content |
| a | 27 | 2.69 | 19 |
| b | 78 | 1.42 | 63 |

### Example 19. Study of Different Fabrics and Finish Compositions on Fibrous Structure Loading

200 g aqueous compositions of 10% lidocaine, with, or without, 10% PEG 400, 10% propylene glycol and 2% Polysorbate 80, were prepared in 250 ml vessels and placed on controlled temperature 400 RPM stirrer plates, warmed to 60-70° C. After 1 hour minutes of stirring, different fabrics were added to each vessel and the temperature allowed to drop to 55-65° C. After a further hour, the samples were removed from their vessels, squeezed, weighed and left to dry at room temperature for at least 16 hours before their dry weight and percentage Add on were determined. The results are shown in Table 14.

TABLE 14

| | | % PEG | |
|---|---|---|---|
| Textile | Denier | 400/PG/PS80 | % Add on |
| 100% Polyamide | 20 | 10/10/2 | 139.96 |
| | | 0 | 203.95 |
| 97% Polyamide, 3% Elastane | 40 | 10/10/2 | 65.73 |
| | | 0 | 131.20 |
| 90% Polyamide, 10% | 20 | 10/10/2 | 85.21 |
| Elastane | | 0 | 190.79 |
| 88% Polyamide, 12% | 40 | 10/10/2 | 71.75 |
| Elastane | | 0 | 134.36 |
| 63% Polyamide, 37% | 15 | 10/10/2 | 161.03 |
| Elastane | | 0 | 175.05 |
| 72% Polyamide, 16% | 40 | 10/10/2 | 103.70 |
| Elastane, | | 0 | 167.54 |
| 12% Polyester | | | |
| 32% Polyamide, 17% | — | 10/10/2 | 110.86 |
| Elastane, | | 0 | 181.74 |
| 51% Cotton* | | | |
| 100% Cotton | — | 10/10/2 | 60.88 |
| | | 0 | 114.73 |

*experiment run on 100 g composition

### Example 20: Study of Fibrous Structure Loading Kinetics

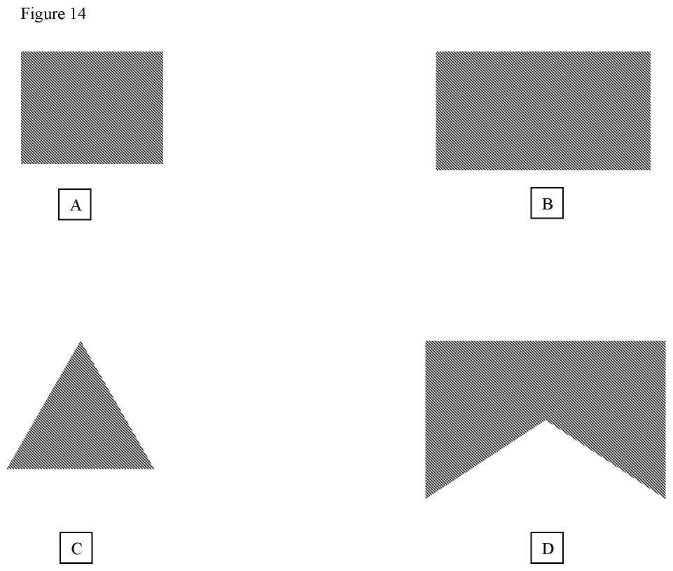

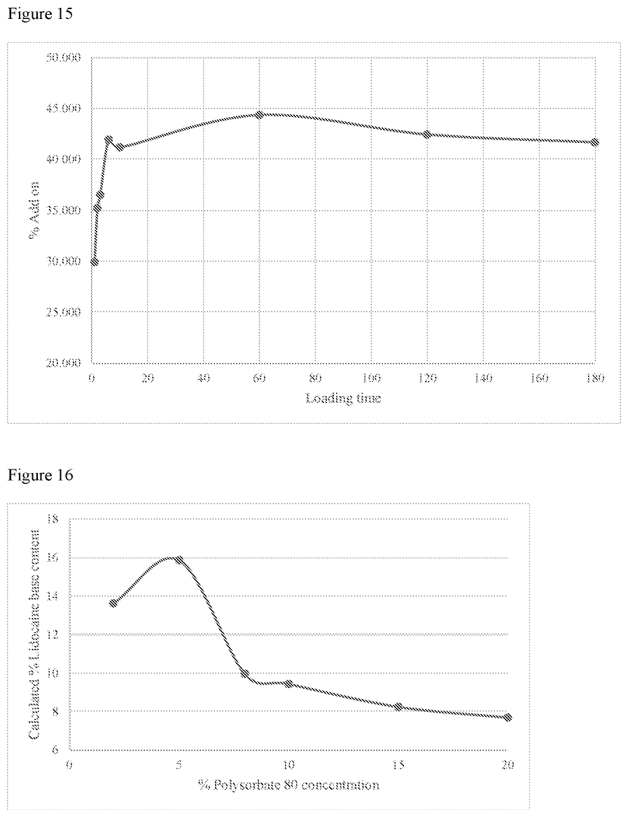

8 different 200 g aqueous compositions containing 10% lidocaine, 10% PEG 400, 10% propylene glycol and 2% Polysorbate 80, were added to 500 ml vessels and placed in a Mathis Labomat, warmed to 60° C. and rotated at 40 PRM. Once the temperature was achieved, pre-cut samples of the shapes, shown in FIGS. 14A-D, of 91.4% polyamide/8.6% elastane were added to each vessel. Vessels were either returned (Group 1, n=5) to the Labomat and mixed at 60° C., or not returned (Group 2, n=3). At time intervals of 1, 2 and 3 minutes, the samples were removed Group 2 vessels and at 6, 10, 60, 120 and 180 minutes from Group 1 vessels. After being removed from the vessels, the samples were squeezed and allowed to dry overnight at room temperature. After drying, the sample's dry weight and percentage Add on were determined. The results are shown in Table 15 and FIGS. 14A, 14B, 14C, 14D, and 15.

TABLE 15

| Loading | % Add on | | | | |
|---|---|---|---|---|---|
| time/min | Combined | Shape A | Shape B | Shape C | Shape D |
| 1 | 29.915 | 29.820 | 27.838 | 30.455 | 31.547 |
| 2 | 35.227 | 34.985 | 36.019 | 34.260 | 35.643 |
| 3 | 36.500 | 37.273 | 36.782 | 35.879 | 36.064 |
| 6 | 41.938 | 42.242 | 44.328 | 40.764 | 40.418 |
| 10 | 41.190 | 39.998 | 44.262 | 40.939 | 39.560 |
| 60 | 44.374 | 45.729 | 41.509 | 48.701 | 41.556 |
| 120 | 42.458 | 42.233 | 43.286 | 43.679 | 40.635 |
| 180 | 41.691 | 42.281 | 40.802 | 42.474 | 41.206 |

### Example 21. Study of API and Finish Composition on Fibrous Structure Loading

8 different aqueous compositions with, or without, 3% lidocaine, 10% PEG 400, 10% propylene glycol and/or 2% Polysorbate 80, were added to 500 ml vessels and placed in a Mathis Labomat, warmed to 60° C. and rotated at 40 PRM. Once the temperature was achieved, samples of 91.4% polyamide/8.6% elastane were added to each vessel. After a further hour, the samples were then either a) left to dry at room temperature for more than 60 hours or b) squeezed and then allowed to dry at room temperature for more than 60 hours. After drying, the sample's dry weight and percentage Add on were determined. The results are shown in Table 16 and Table 17.

TABLE 16

| % (w/w) | | | | Mean % Add | |
|---|---|---|---|---|---|
| | PEG | Propylene | Polysorbate | on (dried | |
| Lidocaine | 400 | glycol | 80 | at RT) | RSD |
| 3 | — | — | — | 37.25 | 22.94 |
| 3 | 10 | — | — | 54.02 | 10.06 |
| 3 | — | 10 | — | 34.89 | 22.05 |
| 3 | — | — | 2 | 19.10 | 0.69 |
| 3 | 10 | 10 | — | 56.37 | 45.06 |
| 3 | 10 | — | 2 | 42.88 | 0.28 |
| 3 | 10 | 10 | 2 | 40.92 | 3.75 |
| 3 | — | 10 | 2 | 19.34 | 0.09 |

TABLE 17

| % (w/w) | | | | | |
|---|---|---|---|---|---|
| | PEG | Propylene | Polysorbate | Mean % Add | |
| Lidocaine | 400 | glycol | 80 | on (squeezed) | RSD |
| 3 | — | — | — | 20.27 | 17.23 |
| 3 | 10 | — | — | 32.03 | 5.51 |
| 3 | — | 10 | — | 21.14 | 8.27 |
| 3 | — | — | 2 | 14.97 | 3.00 |
| 3 | 10 | 10 | — | 30.01 | 15.54 |
| 3 | 10 | — | 2 | 24.91 | 2.54 |
| 3 | 10 | 10 | 2 | 25.73 | 0.95 |
| 3 | — | 10 | 2 | 14.48 | 1.98 |
| — | — | — | — | −0.17 | −63.70 |
| — | 10 | — | — | 13.07 | 3.20 |
| — | — | 10 | — | −0.28 | −19.83 |
| — | — | — | 2 | 1.96 | 9.47 |
| — | 10 | 10 | — | 13.58 | 0.29 |
| — | 10 | — | 2 | 13.41 | 2.34 |
| — | 10 | 10 | 2 | 15.04 | 5.63 |
| — | — | 10 | 2 | 2.19 | 27.08 |

### Example 22. Study of Humectant and Permeation Enhancer Concentration on Fibrous Structure Loading

Different 200 g aqueous compositions containing 2-20% PEG 400 or propylene glycol, with, or without, 3% lidocaine and 2% Polysorbate 80, were added to 500 ml vessels and placed in a Mathis Labomat, warmed to 60° C. and rotated at 40 PRM. Once the temperature was achieved, samples of 91.4% polyamide/8.6% elastane were added to each vessel. After a further hour, the samples were then squeezed and then allowed to dry at room temperature for more than 60 hours. After drying, the sample's dry weight and percentage Add on were determined. The results are shown in Table 18.

TABLE 18

| % (w/w) | | | | | |
|---|---|---|---|---|---|
| | PEG | Propylene | Polysorbate | Mean | |
| Lidocaine | 400 | glycol | 80 | % Add on | RSD |
| — | 2 | — | — | 2.48 | 15.28 |
| — | 8 | — | — | 7.84 | 5.21 |
| — | 15 | — | — | 15.20 | 0.29 |
| — | 20 | — | — | 20.06 | 1.57 |
| — | — | 2 | — | 2.59 | 2.34 |
| — | — | 8 | — | 7.43 | 6.32 |
| — | — | 15 | — | 12.72 | 0.09 |
| — | — | 20 | — | 19.14 | 0.06 |
| 3 | 2 | — | 2 | 16.59 | 0.95 |
| 3 | 8 | — | 2 | 26.06 | 2.15 |
| 3 | 15 | — | 2 | 30.96 | 2.69 |
| 3 | 20 | — | 2 | 37.95 | 5.14 |
| 3 | — | 2 | 2 | 14.96 | 1.49 |
| 3 | — | 8 | 2 | 15.93 | 2.29 |
| 3 | — | 15 | 2 | 14.84 | 0.26 |
| 3 | — | 20 | 2 | 16.03 | 3.45 |

### Example 23. Study of Surfactant Concentration on Fibrous Structure Loading

6 different aqueous compositions containing 3% lidocaine together with 2 to 20% Polysorbate 80 or 10% PEG 400 or 10% propylene glycol, were added to 500 ml vessels and placed in a Mathis Labomat, warmed to 60° C. and rotated at 40 PRM. Once the temperature was achieved, samples of 91.4% polyamide/8.6% elastane were added to each vessel. After a further hour, the samples were then squeezed and then allowed to dry at room temperature for 68 hours. After drying, the sample's dry weight and percentage Add on were determined. The results are shown in Table 19.

TABLE 19

| % (w/w) | | | | Mean % Add | |
|---|---|---|---|---|---|
| | PEG | Propylene | Polysorbate | on (dried | |
| Lidocaine | 400 | glycol | 80 | at RT) | RSD |
| 3 | — | — | 2 | 16.18 | 1.54 |
| 3 | — | — | 5 | 22.89 | 0.90 |
| 3 | — | — | 8 | 21.18 | 2.57 |
| 3 | — | — | 10 | 24.11 | 3.00 |
| 3 | — | — | 15 | 33.29 | 1.26 |
| 3 | — | — | 20 | 38.08 | 6.97 |
| 3 | 10 | — | — | 39.67 | 13.44 |
| 3 | — | 10 | — | 23.99 | 28.13 |

The mean lidocaine base content on the sample was calculated for each of the Polysorbate 80 samples and the results shown in FIG. 16.

### Example 24. Study of pH on Fibrous Structure Loading

Aqueous compositions of 200 g of 10% lidocaine (as the HCl salt), 10% PEG 400, 10% propylene glycol and 2% Polysorbate 80 with their pH tittered by the inclusion of KOH, were added to 500 ml vessels and placed in a Mathis Labomat, warmed to 60° C. and rotated at 40 PRM. After an hour the vessels were removed from the Labomat and samples of 91.4% polyamide/8.6% elastane was added to each vessel at a weight ratio of 1:10, sample:loading composition. Immediately prior to the samples being removed, each loading composition was sampled and the concentration of lidocaine assayed by HPLC. The results are shown in Table 20.

TABLE 20

| | | Concentration |
|---|---|---|
| | | of loading |
| | | composition |
| | Achieved pH | % w/w |
| Loading composition 1 | <0 | 9.93 |
| Loading composition 2 | 2.61 | 9.88 |
| Loading composition 3 | 5.55 | 9.58 |
| Loading composition 4 | 6.18 | 7.44 |
| Loading composition 5 | 7.01 | 6.79 |
| Loading composition 6 | 8.94 | 6.77 |
| Loading composition 7 | 11.26 | 7.79 |
| Loading composition 8 | >14 | 4.53 |

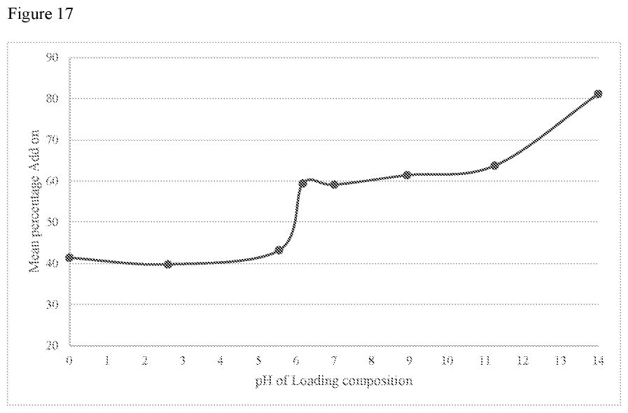

After a further hour, the samples were removed from their vessels, squeezed, weighed and left to dry at room temperature for a further 12 hours before their dry weight and percentage Add on were determined. The results are shown in FIG. 17.

### Example 25: Study of Fibrous Structure Loading Kinetics and Dissolution

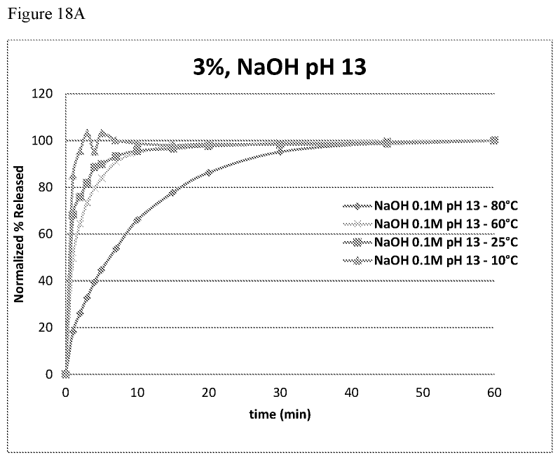

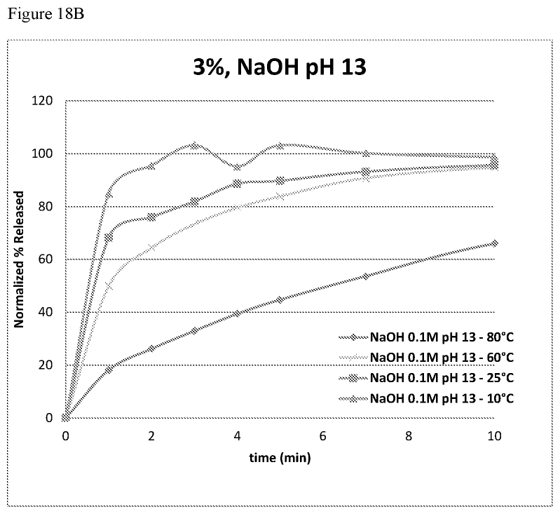

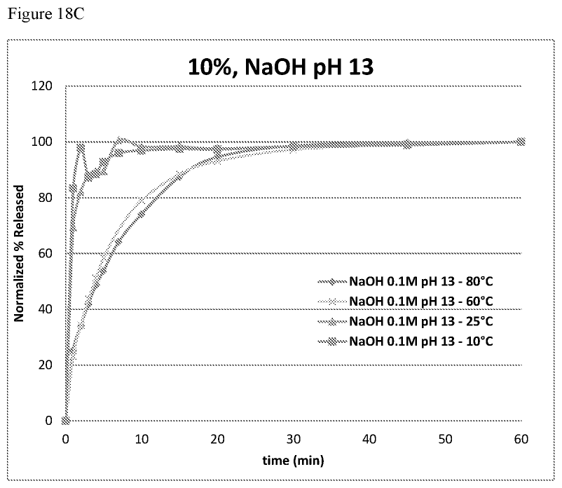

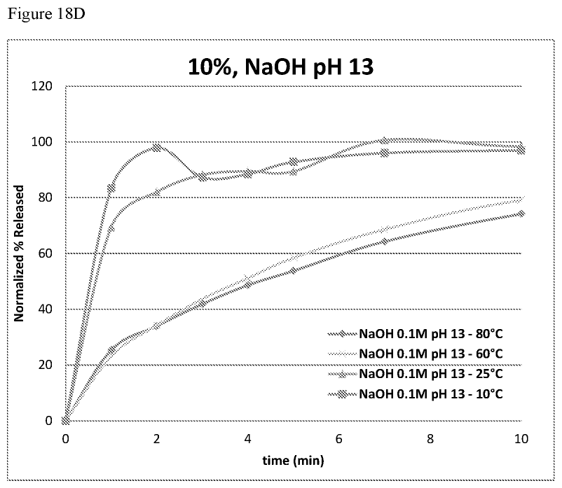

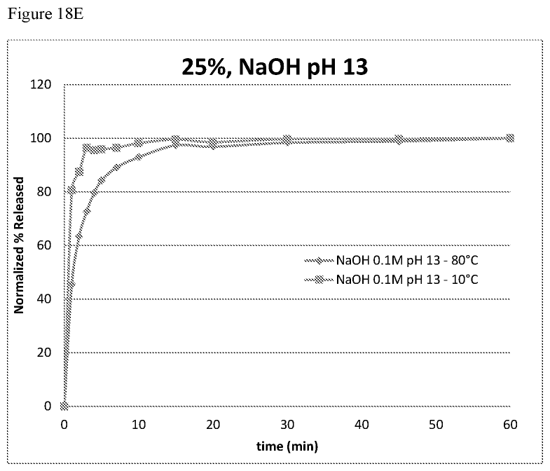

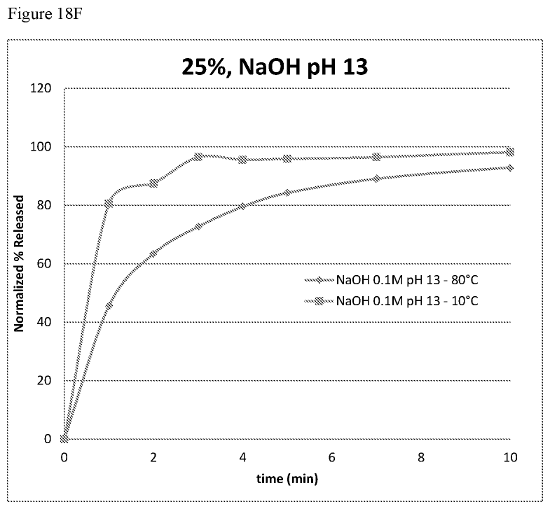

200 g aqueous compositions with 10% PEG 400, 10% propylene glycol, 2% Polysorbate 80 and 3%, 10% or 25% lidocaine, were added to 500 ml vessels and mixed to a controlled temperature. Vessels to be controlled at <10° C. were placed in an ice bath and mixed with a mechanical stirrer for at least 30 minutes. Vessels to be controlled at 60° C. or 80° C. were placed in a Mathis Labomat, warmed to either 60 or 80° C. and rotated at 40 RPM for at least 30 minutes. Once the temperatures were achieved, 20 g samples of 91.4% polyamide/8.6% elastane were added to each vessel. After a further hour, the samples were then squeezed and then allowed to dry overnight at room temperature. After drying, the sample's dry weight and percentage Add on were determined. Using assumptions of 8% (w/w) loading for the 3% lidocaine sample, 25% (w/w) loading for the 10% lidocaine sample and 50% (w/w) loading for the 25% lidocaine sample, 0.5 g, 1 g and 3 g pieces respectively, of the samples were cut off and introduced to a beaker containing 500 ml of 0.1M NaOH (pH 13) and stirred with a mechanical stirrer at 450 RPM. The media was sampled directly by HPLC ((Luna C18(2), 100 Å, 5 μm, 250×4.6 mm (Phenomenex), mobile phase—80% water with acetic acid (pH 3.4) and 20% Acetonitrile, UV detection at 254 nm at room temperature, flow rate of 1.5 mL/min)) at 1, 2, 3, 4, 5, 7, 10, 15, 20, 30, 45 and 60 minutes. The results are shown in FIGS. 18A-18F.

### Example 26. Study of Leveling of Fibrous Structure Loading

200 ml aqueous compositions of 10% lidocaine, 10% PEG 400, 10% propylene glycol and 2% Polysorbate 80, were added to 500 ml vessels and placed in a Mathis Labomat, warmed to 60° C. and rotated at 40 PRM. Once the temperature was achieved, samples of 91.4% polyamide/8.6% elastane were added to each vessel. After a further hour, the samples were then squeezed, weighed and allowed to dry at room temperature for more than 60 hours. Samples were then added to 5 separate 20 ml vessels each containing 10 gr 0.01M HCl and placed on 400 RPM, stirrer plates at room temperature. Each sample was mixed for between 1 and 22 hours and then removed, rinsed in about 11 of deionized water and left to dry for 8 hours at room temperature before being weighed. The concentration of lidocaine remaining in each vessel was then identified using HPLC (Luna C18(2), 100 Å, 5 μm, 250×4.6 mm (Phenomenex), mobile phase—80% water with acetic acid (pH 3.4) and 20% Acetonitrile, UV detection at 254 nm at room temperature, flow rate of 1.5 mL/min) and the % lidocaine Add on of each sample calculated. The results are shown in Table 21.

TABLE 21

| Exposure to | % lidocaine | |
|---|---|---|
| 0.01M HCl/hr | Add on | RSD |
| 1 | 18.86 | 1.91% |
| 2 | 18.07 | |
| 4 | 18.78 | |
| 6 | 18.25 | |
| 22 | 18.04 | |

### Example 27. Study of Fibrous Structure Air Permeability

Samples of 91.4% polyamide/8.6% elastane and 100% cotton were loaded, as per the method described in Example 26, with aqueous finishing compositions of lidocaine at concentrations between 0.1% to 20%, together with, or without, 10% PEG 400, 10% propylene glycol and 2% Polysorbate 80. The samples were subsequently weighed and left to dry overnight at room temperature before their dry weight and percentage Add on were determined.

Using a modified ASTM D737-09 methodology, the sample's air permeability was tested using a YG461E/II Digital Fabric Air Permeability Tester (Ningbo Textile Instrument Factory). The samples were placed in an atmospheric environment of 25° C. temperature, and 50±2% relative humidity with a test area of 20 cm². The test pressure drop was set to 200 Pa. and nozzles used were 4, 6 (for polyamide samples), and 12 (for cotton samples) and values were measures as the permeation rate of volume (mm/sec). The results are shown in Table 22.

TABLE 22

| Sample | % Add on | Air Permeability mm/sec |
|---|---|---|
| 91.4% polyamide/ | 0 | 364 |
| 8.6% elastane | 17 | 409 |
| | 39 | 333 |
| | 52 | 231 |
| | 69 | 366 |
| 100% Cotton | 16 | 2188 |
| | 20 | 2356 |
| | 33 | 2422 |
| | 61 | 2322 |
| | 91 | 2519 |
| | 138 | 1980 |

### Example 28. Study of Fibrous Structure Stiffness

Samples of 91.4% polyamide/8.6% elastane and 100% cotton were loaded, as per the method described in Example 26, with aqueous finishing compositions of lidocaine at concentrations between 0.1% to 20%, together with, or without, 10% PEG 400, 10% propylene glycol and 2% Polysorbate 80. The samples were subsequently weighed and left to dry overnight at room temperature before their dry weight and percentage Add on were determined.

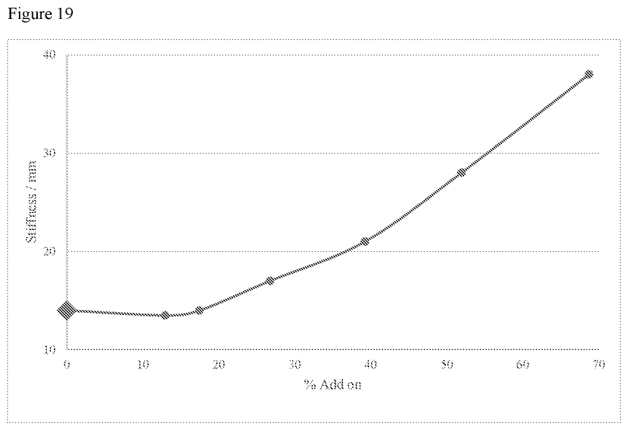

The flexural rigidity of the samples was evaluated using a Shirley Stiffness Tester to measure bending length in millimeters, using a metal ruler to a standard angle of 41.50°. The stiffness test was performed according to ASTM method D1388-18 (Standard Test Method for Stiffness of Fabrics). The bending length was directly obtained by sliding a strip of the sample (2.5 cm wide and 5 cm long) out of the smooth low friction, flat surface platform and letting the fabric tip drape to the plane with a deflection angle below the horizontal of 41.5°. The length of fabric required to bend to this angle is recorded. The greater the length, the greater the fabric's resistance to bend. The results are shown in Table 23 and FIG. 19.

TABLE 23

| Sample | % Add on | Stiffness/mm |
|---|---|---|
| 91.4% polyamide/ | 0 | 14 |
| 8.6% elastane | 13 | 14 |
| | 17 | 14 |
| | 27 | 17 |
| | 39 | 21 |
| | 52 | 28 |
| | 69 | 38 |
| 100% Cotton | 0 | 22 |
| | 16 | 21 |
| | 20 | 21 |
| | 33 | 26 |
| | 61 | 36 |
| | 92 | 39 |
| | 138 | 68 |

### Example 29. Study of Fibrous Structure Tensile Properties

Samples of 91.4% polyamide/8.6% elastane or 100% cotton were loaded, as per the method described in Example 26, with aqueous finishing compositions of lidocaine at concentrations between 0.1% to 20%, together with, or without, 10% PEG 400, 10% propylene glycol and 2% Polysorbate 80. The samples were subsequently weighed and left to dry overnight at room temperature before their dry weight and percentage Add on were determined.

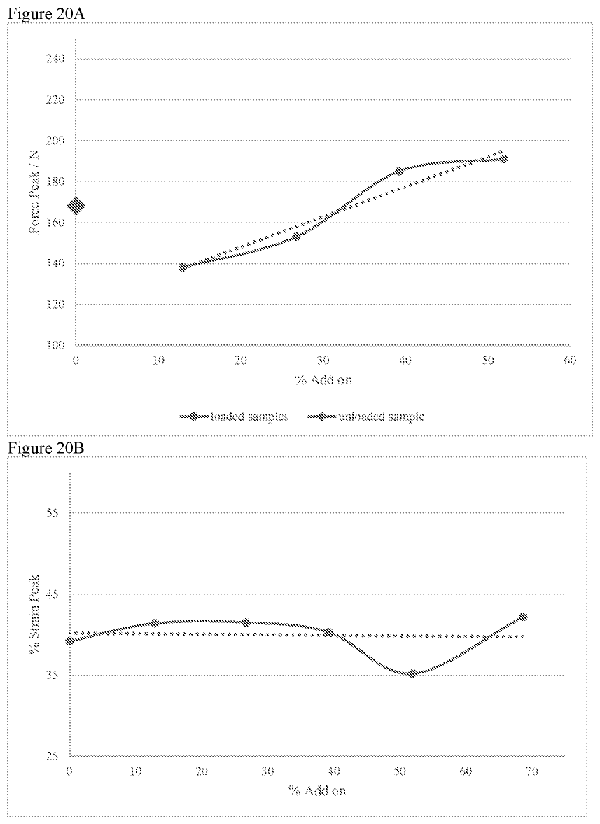

Using a Testometric Tensile Testing Machine M350-10CT (Testometric Co. Ltd., Rochdale, Lancs., England) with a maximum load capacity of 10 kN and according to ASTM D5034-09(2017) test method, the samples' tensile strength and elongation at break (%) were assayed. With all measurements undertaken at 25° C. and 51% RH, the samples were cut into 10 cm×5 cm strips, held with an initial grip separation of 2.5 cm and then pulled apart at a head speed of 300 mm/min. The Force at peak and Strain at peak were recorded by Win-Test® analysis software v4.4.5. The results are shown in Table 24 and FIGS. 20A and 20B.

TABLE 24

| Sample | % Add on | Force Peak/N | % Strain Peak |
|---|---|---|---|
| 91.4% polyamide/ | 0 | 168 | 39 |
| 8.6% elastane | 13 | 138 | 41 |
| | 17 | 188 | 49 |
| | 27 | 153 | 42 |
| | 39 | 185 | 40 |
| | 52 | 191 | 35 |
| | 69 | 227 | 39 |
| 100% Cotton | 0 | 217 | 19 |
| | 16 | 247 | 21 |
| | 20 | 245 | 25 |
| | 33 | 261 | 22 |
| | 61 | 206 | 24 |
| | 92 | 196 | 25 |
| | 138 | 226 | 23 |

### Example 30. Study of Fibrous Structure Moisture Vapor Transmission Rate

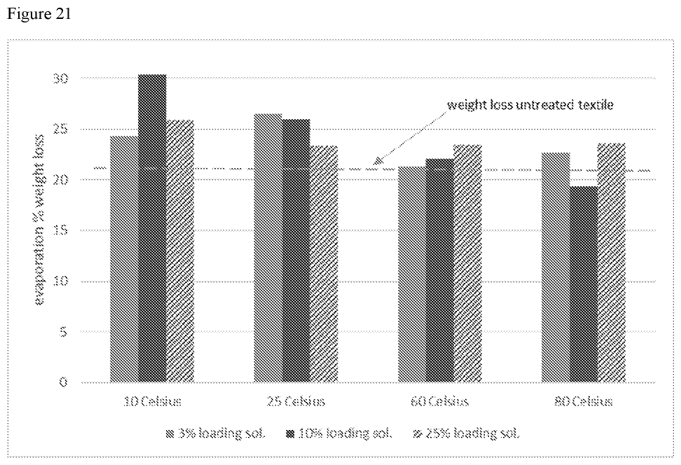

200 g aqueous compositions with 10% PEG 400, 10% propylene glycol, 2% Polysorbate 80 and 3%, 10% or 25% lidocaine, were added to 500 ml vessels and mixed to a controlled temperature. Vessels to be controlled at <10° C. were placed in an ice bath and mixed with a mechanical stirrer for at least 30 minutes. Vessels to be controlled at 60° C. or 80° C. were placed in a Mathis Labomat, warmed to either 60 or 80° C. and rotated at 40 RPM for at least 30 minutes. Once the temperatures were achieved, 20 g samples of 91.4% polyamide/8.6% elastane were added to each vessel. After a further hour, the samples were then squeezed and then allowed to dry overnight at room temperature. After drying, the sample's dry weight and percentage Add on were determined. Using a modified ASTM E96-80 methodology, 200 ml of water was added to 500 ml beakers (diameter 75 mm) and closed with the samples, as described above, firmly fixed on top. The beakers were left at 40° C. for 72 hours before being weighed and their percentage evaporation weight loss calculated. The results are shown in FIG. 21.

What is claimed:

1. A method of treating a condition of a subject comprising applying to the intact skin of a body part of the subject, an item of clothing, wherein the item of clothing comprises:
a dry, non-occlusive, fibrous structure finished with a finishing composition that is a homogeneous dispersion comprising an active pharmaceutical ingredient, a surfactant, and a solvent;
wherein the condition is a painful condition, an inflammatory condition, an infective condition, or a dermatological condition; and
wherein the application is for a time period and provides topical administration of a therapeutically effective amount of the active pharmaceutical ingredient, locally and/or systemically, to the subject.

2. The method of claim 1, wherein the item of clothing is a sock, a knee brace, a shoulder brace, an elbow sleeve, a wrist band, a glove, a neck sleeve, a back brace, a sleeve, a t-shirt, or a bandage.

3. The method of claim 1 or claim 2, wherein the item of clothing is a sock and the sock is a toed sock, a toeless sock or a non-toed sock.

4. The method of claim 3, wherein the item of clothing is a sock and the sock is an ankle sock, a mid-length sock, a knee-length sock or a high length sock.

5. The method of any one of the preceding claims, wherein the item of clothing is a sock and the sock is a toed ankle sock, a toeless ankle sock, a non-toed ankle sock, a toed mid-length sock, a toeless mid-length sock, a non-toed mid-length sock, a toed knee-length sock, a toeless knee-length sock, a non-toed knee-length sock, a toed high-length sock, a toeless high-length sock or a non-toed high-length sock.

6. The method of claim 1 or claim 2, wherein the item of clothing is a knee brace.

7. The method of claim 1 or claim 2, wherein the item of clothing is a shoulder brace.

8. The method of claim 1 or claim 2, wherein the item of clothing is an elbow sleeve.

9. The method of claim 1 or claim 2, wherein the item of clothing is a wrist band.

10. The method of claim 1 or claim 2, wherein the item of clothing is a glove and the glove is a short glove or an elbow glove.

11. The method of claim 10, wherein the glove is a finger glove, a fingerless glove, or a mitten.

12. The method of claim 10 or claim 11, wherein the glove is a finger short glove, finger elbow glove, fingerless short glove, fingerless elbow glove, short glove mitten or elbow glove mitten.

13. The method of claim 1 or claim 2, wherein the item of clothing is a neck sleeve and the neck sleeve is a closed neck sleeve or a scarf.

14. The method of claim 1 or claim 2, wherein the item of clothing is a back brace.

15. The method of claim 1 or claim 2, wherein the item of clothing is a sleeve.

16. The method of claim 1 or claim 2, wherein the item of clothing is a t-shirt and the t-shirt is a short-shirt or a long shirt.

17. The method of claim 1 or claim 2, wherein the item of clothing is a bandage.

18. The method of any one of the preceding claims, wherein the condition is a painful condition.

19. The method of claim 18, wherein the painful condition is superficial nociceptive pain, deep nociceptive pain, superficial and deep nociceptive pain, central neuropathic pain, peripheral neuropathic pain, or mixed central and peripheral neuropathic pain.

20. The method of claim 18 or claim 19, wherein the painful condition is superficial nociceptive pain, for example, pain associated with abrasions, cuts, bruises, burns, sunburns, or insect bites.

21. The method of claim 18 or claim 19, wherein the painful condition is deep nociceptive pain, for example, musculoskeletal pain (e.g., pain associated with sprains, tears to ligaments, tendons or muscle tissue, bone fractures, inflammation of tendons, joints, bursae or fascia and degenerative changes to the musculoskeletal system), post-operative pain, or inflammatory response related pain.

22. The method of claim 18 or claim 19, wherein the painful condition is central neuropathic pain, for example, pain associated with a central venous thrombosis, pain associated with a cerebral tumor, pain associated with a traumatic brain lesion, pain associated with multiple sclerosis, pain associated with Parkinson's disease, pain associated with the pain occurring after a stroke or a thalamotomy, pain associated with a spinal cord injury, pain associated with complications of spinal surgery, pain associated with ischemic lesions of the spinal cord, pain associated with radiation, or pain associated with HIV associated myelopathy.

23. The method of claim 18 or claim 19, wherein the painful condition is peripheral neuropathic pain (e.g., pain associated with lesions to peripheral nerves, lesions to nerve roots and posterior ganglia and lesions to cranial nerves), pain associated with compressions to peripheral nerves, pain associated with compressions to nerve plexuses, pain associated with compressions to and nerve roots, pain associated with length dependent neuropathies, pain associated with those lesions caused by systemic disease (e.g., diabetic neuropathy, lupus neuropathy or hypothyroidism associated neuropathy), pain associated with drug-induced disorders (e.g., chemotherapy induced neuropathy, metabolic or nutritional disorders such as alcoholic neuropathy, traumatic and entrapment syndromes), pain associated with post-surgical nerve injuries, pain associated with inflammatory demyelinating polyradiuloneuropathy, pain associated with HIV sensory neuropathy, pain associated with those lesions associated with postherpetic neuralgia and nerve root avulsions (e.g., pain associated with cranial neuralgias such as trigeminal neuralgia), pain associated with paraneoplastic peripheral neuropathy, pain associated with ganglionopathy, pain associated with complications of chemotherapy, pain associated with radiotherapy, pain associated with surgery, pain associated with Type 1 Complex Regional Pain Syndrome, or pain associated with Type 2 Complex Regional Pain Syndrome.

24. The method of any one of the preceding claims, wherein the condition is an inflammatory condition.

25. The method of claim 24, wherein the inflammatory condition is a locally manifesting inflammatory condition or a systemic inflammatory condition.

26. The method of claim 24 or claim 25, wherein the inflammatory condition is bursitis, dermatomyositis, lichen planus, neuritis, panniculitis, phlebitis, tendonitis, or tendinosis.

27. The method of claim 24 or claim 25, wherein the inflammatory condition is an autoimmune disease (e.g., ankylosing spondylitis, juvenile idiopathic arthritis (JIA), mast cell activation syndrome (MCAS), psoriasis, reactive arthritis, rheumatoid arthritis, Sjögren's syndrome or systemic lupus erythematosus (SLE)), gout, inflammatory bowel disease, pseudogout, rheumatic fever, sarcoidosis, or vasculitis.

28. The method of any one of the preceding claims, wherein the condition is an infective condition.

29. The method of claim 28, wherein the infective condition is a mycotic infection (e.g., tinea versicolor, tinea corporis, tinea pedis or onychomycosis), a bacterial infection (e.g., folliculitis, impetigo, erysipelas, cellulitis, or infections associated with minor cuts, scrapes or burns), a parasitic infection (e.g., scabies, leishmaniasis, or hookworm), a viral infection (e.g., a varicella zoster virus infection or a human papillomavirus infection.

30. The method of any one of the preceding claims, wherein the condition is a dermatological condition.

31. The method of claim 30, wherein the dermatological condition is an allergic condition (e.g., atopic dermatitis, contact dermatitis or urticaria), a cutaneous malignancy (e.g., cutaneous t-cell lymphomas, superficial basal cell carcinomas or AIDS-related Kaposi's sarcoma), a blister (e.g., dermatitis herpetiformis, pemphigus or pemphigoid), pruitis, acneiform eruption, keratosis (e.g., actinic keratosis or hyperkeratosis), a cutaneous condition secondary to an external cause, or a corticosteroid-responsive dermatoses.

32. The method of any one of the preceding claims, wherein the condition is a painful condition and the active pharmaceutical ingredient is articaine, benzocaine, bupivacaine, butamben, cinchocaine, chloroprocaine, cyclomethycaine, dibucaine, dimethisoquin, dyclonine, etidocaine, levobupivacaine, lidocaine, mepivacaine, pramoxine, piperocaine, prilocaine, propoxycaine, procaine, proparacaine, ropivacaine, tetracaine, trimecaine, aceclofenac, aspirin, benzydamine, celecoxib, dexketoprofen, diflunisal, droxicam, dexibuprofen, diclofenac, enolic acid, etodolac, etoricoxib, fenoprofen, firocoxib, flufenamic acid, flurbiprofen, ibuprofen, indomethacin, ketoprofen, ketorolac, lornoxicam, loxoprofen, meclofenamic acid, mefenamic acid, meloxicam, nabumetone, naproxen, nimesulide, oxaprozin, paracetamol, parecoxib, phenylbutazone, piroxicam, salsalate, sulindac, tenoxicam, tolmetin, tolfenamic acid, carbamazepine, gabapentin, lacosamide, lamotrigine, levetiracetam, oxcarbazepine, pregabalin, tiagabine, valproate, vigabatrin, capsaicin, menthol, clonidine, detomidine, dexmedetomidine, duloxetine, medetomidine, brimonidine, guanabenz, camphor, amitriptyline, baclofen, benfotiamine, capsaicin, citalopram, desipramine, doxepine, dronabinol, duloxetine, escitalopram, fluoxetine, mexiletine, naloxone, paroxetine, sertraline, thioctic acid, trazodone or a combination thereof.

33. The method of any one of the preceding claims, wherein the condition is an inflammatory condition and the active pharmaceutical ingredient is an anti-histamine (e.g., acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine, or triprolidine), an anti-leukotriene (e.g., curcumin, montelukast, pranlukast, St. John's wort, zafirlukast, or zileuton), an anti-metabolite (e.g., azathioprine, leflunomide, mercaptopurine, methotrexate, mycophenolic acid, pemetrexed or pralatrexate), a corticosteroid (e.g., alclometasone, amcinonide, betamethasone, clobetasol, clocortolone, desonide, desoximetasone, diflorasone, fluocinolone, flurandrenolide, fluticasone, halcinonide, halobetasol, hydrocortisone, prednicarbate, triamcinolone, or mometasone), a disease-modifying antirheumatic drug (DMARD) (e.g., azathioprine, chloroquine, ciclosporin, gold salts, hydroxychloroquine, leflunomide, mesalazine, methotrexate, sulfasalazine or tofacitinib), an immunomodulator (e.g., apremilast, azathioprine, lenalidomide, methotrexate, pentoxifylline, pomalidomide or thalidomide), an immunosuppressant (e.g., azathioprine, cyclophosphamide, cladribine, chlorambucil, cyclophosphamide, cyclosporine, everolimus, mercaptopurine, methotrexate, mycophenolic acid, sirolimus or tacrolimus), a non-steroidal anti-inflammatory drug (NSAID) (e.g., aceclofenac, aspirin, benzydamine, celecoxib, dexketoprofen, diflunisal, droxicam, dexibuprofen, diclofenac, enolic acid, etodolac, etoricoxib, fenoprofen, firocoxib, flufenamic acid, flurbiprofen, ibuprofen, indomethacin, ketoprofen, ketorolac, lornoxicam, loxoprofen, meclofenamic acid, mefenamic acid, meloxicam, nabumetone, naproxen, nimesulide, oxaprozin, paracetamol, parecoxib, phenylbutazone, piroxicam, salsalate, sulindac, tenoxicam, tolmetin, tolfenamic acid, acitretin, adapalene, alitretinoin, allopurinol, apremilast, an ACE inhibitor, bexarotene, calcipotriol, chloroquine, coal tar, colchicine, cromolyn sodium, dapsone, etretinate, febuxostat, hydroxychloroquine, isotretinoin, minocycline, paricalcitol, pentoxifylline, potassium iodide, probenecid, roflumilast, salicylic acid, sulfasalazine, tazarotene or ursodeoxycholic acid or a combination thereof.

34. The method of any one of the preceding claims, wherein the condition is an infective condition and the active pharmaceutical ingredient is bacitracin, benzoyl peroxide, cephalexin, ciprofloxacin, clindamycin, dapsone, erythromycin, loxacillin, dicloxacillin, flucloxacillin, linezolid, minocycline, mupirocin, neomycin, polymyxin B, rifampin, trimethoprim-sulfamethoxazole, amoxicillin-clavulanate potassium, bacitracin, cephalexin, chloramphenicol, ciprofloxacin, clindamycin, doxycycline, erythromycin, fusidic acid, gentamicin, levofloxacin, linezolid, mupirocin, neosporin, ofloxacin, ozenoxacin, retapamulin, tetracycline, or trimethoprim-sulphamethoxazole, ampicillin, amoxicillin, amoxicillin-clavulanate potassium, azithromycin, azlocillin, bacampicillin, carbenicillin, cephalexin, clindamycin, cloxacillin, dicloxacillin, epicillin, erythromycin, flucloxacillin, hetacillin, metampicillin, methicillin, mezlocillin, nafcillin, penicillin, piperacillin, pivampicillin, oxacillin, sulbactam, talampicillin, tazobactam, temocillin, ticarcillin, amoxicillin, ampicillin-sulbactam, azithromycin, cefuroxime, cephalexin, ceftriaxone, cefazolin, clarithromycin, clindamycin, dicloxacillin, erythromycin, levofloxacin, nafcillin, oxacillin, penicillin, vancomycin, or bacitracin, chlortetracycline, neomycin, tetracycline, butenafine, selenium, terbinafine, betamethasone, butenafine, ciclopirox, clioquinol, clotrimazole, econazole, haloprogin, luliconazole, miconazole, naftifine, oxiconazole, povidone-iodine, sertaconazole, sulconazole, terbinafine, tolnaftate, undecylenic acid, betamethasone, butenafine, ciclopirox, clioquinol, clotrimazole, econazole, haloprogin, luliconazole, miconazole, naftifine, oxiconazole, povidone-iodine, sertaconazole, sulconazole, terbinafine, tolnaftate, undecylenic acid, benzyl benzoate, crotamiton, ivermectin, lindane, moxidectin, permethrin, precipitated sulfur, salicylic acid, synergized pyrethrins, allopurinol, amphotericin B, antimonite, cotrimoxazole, dapsone, fluconazole, ketoconazole, meglumine, methylbenzethonium chloride, metronidazole, miltefosine, paromomycin, pentoxifylline, raconazole, rifampicin, sodium stibogluconante, albendazole, levamisole, mebendazole, pyrantel pamoate, acyclovir, famciclovir, penciclovir, valacyclovir, benzoyl peroxide, bleomycin, cantharidin, dinitrochlorobenzene, formic acid, imiquimod, podophyllin, salicylic acid, silver nitrate, trichloroacetic acid, zinc oxide, zinc sulfate or a combination thereof.

35. The method of any one of the preceding claims, wherein the condition is a dermatological condition and the active pharmaceutical ingredient is an anti-histamine (e.g., acrivastine, azelastine, bepotastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, ketotifen, levocabastine, levocetirizine, loratadine, meclizine, mirtazapine, mizolastine, olopatadine, orphenadrine, phenindamine, pheniramine, phenyltoloxamine, promethazine, pyrilamine, rupatadine, tripelennamine or triprolidine), an anti-leukotriene (e.g., curcumin, montelukast, pranlukast, St. John's wort, zafirlukast or zileuton), a corticosteroid (e.g., alclometasone, amcinonide, betamethasone, clobetasol, clocortolone, desonide, desoximetasone, diflorasone, fluocinolone, flurandrenolide, fluticasone, halcinonide, halobetasol, hydrocortisone, prednicarbate, triamcinolone, or mometasone), an immunomodulator (e.g., apremilast, azathioprine, lenalidomide, methotrexate, pentoxifylline, pomalidomide or thalidomide), an immunosuppressant (e.g., azathioprine, cyclophosphamide, cladribine, chlorambucil, cyclophosphamide, cyclosporine, everolimus, mercaptopurine, methotrexate, mycophenolic acid, pimecrolimus, sirolimus or tacrolimus), a local anesthetic (articaine, benzocaine, bupivacaine, butamben, cinchocaine, chloroprocaine, cyclomethycaine, dibucaine, dimethisoquin, dyclonine, etidocaine, levobupivacaine, lidocaine, mepivacaine, pramoxine, piperocaine, prilocaine, propoxycaine, procaine, proparacaine, ropivacaine, tetracaine or trimecaine), or a topical analgesic drug (e.g, capsaicin, menthol, clonidine, detomidine, dexmedetomidine, duloxetine, medetomidine, brimonidine, guanabenz, camphor, abrocitinib, acitretin, adapalene, alitretinoin, aminolevulinic acid, baricitinib, bexarotene, bleomycin, calcipotriol, capsaicin, carmustine, cholestyramine, colchicine, crisaborole, cromolyn sodium, dapsone, denileukin, desonide, dexpanthenol, diclofenac, diftitox, doxepin, doxorubicin, erythromycin, estradiol, etoposide, etretinate, fluorouracil, imiquimod, ingenol, isothipendyl, isotretinoin, lactic acid, lymecycline, maxacalcitol, mechlorethamine, methotrexate, nalfurafine, nicotinamide, pseudoephedrine, romidepsin, ruxolitinib salicylic acid, serlopitant, sonidegib, sulfamethoxypyridazine, sulfapyridine suplatast, sulfasalazine, tazarotene, tetracycline, tradipitant, tretinoin, verapamil, vorinostat, or vismodegib or a combination thereof.

36. The method of any one of the preceding claims, wherein time period is from about 1 hour to about 168 hours.

37. The method of claim 35, wherein the time period is about 1 hour to about 3 hours, 1 hour to about 6 hours, 1 hour to about 8 hours, 1 hour to about 12 hours, 1 hour to about 24 hours, 1 hour to about 48 hours, 1 hour to about 72 hours, 1 hour to about 96 hours, 1 hour to about 120 hours, or 1 hour to about 144 hours.

38. The method of any one of the preceding claims, wherein the body part is a foot, a knee, a shoulder, an elbow, a wrist, a hand, neck, back, body appendage, a torso or a combination thereof.

39. The method of any one of the preceding claims, wherein the topical administration of the therapeutically effective amount of the active pharmaceutical ingredient is locally administered to the subject.

40. The method of any one of the preceding claims, wherein the topical administration of the therapeutically effective amount of the active pharmaceutical ingredient is systemically administered to the subject.

41. The method of any one of the preceding claims, wherein the topical administration of the therapeutically effective amount of the active pharmaceutical ingredient is locally and systemically administered to the subject.
